SEQUENCE LISTING (16 sequences)

SEQ ID NO: 1 - protein, 735 aa, unidentified [sequence omitted]

SEQ ID NO: 2 - protein, 735 aa, synthetic construct
MAADGYLPDWLEDTLSEGIRQWWKLKPGPPPPKPAERHQDDSRGLVLPGYKYLGPFNGLDKGEPVSEADVAALEHDKAYDRQLDSGDNPYLKYNHADAEFQERLKEDTSFGGNLGRAVFQAKKRVLEPLGLVEEPVKTAPGKKRPVEHSPAEPDSSSGTGKAGQQPARKRLNFGQTGDADSVPDPQPLGQPPAAPSGLGTNTMATGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSKTNAPSGTTTMSRLQFSQAGASDIRDQSRNWLPGPCYRQQRVSKTAADNNNSDYSWTGATKYHLNGRDSLVNPGPAMASHKDDEEKYFPQSGVLIFGKQDSGKTNVDIEKVMITDEEEIRTTNPVATEQYGSVSTNLQSGNTQAATTDVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRYLTRNL

SEQ ID NO: 3 - protein, 735 aa, synthetic construct
MAADGYLPDWLEDTLSEGIRQWWKLKPGPPPPKPAERHQDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPGKKRPVEHFPAEPDSSSGTGKAGQQPARKRLNFGQTGDADSVPDPQPLGQPPAAPSGLGTNTMATGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSKTNAPSGTTTMSRLQFSQAGASDIRDQSRNWLPGPCYRQQRVSKTAADNNNSDYSWTGATKYHLNGRDSLVNPGPAMASHKDDEEKYFPQSGVLIFGKQDSGKTNVDIEKVMITDEEEIRTTNPVATEQYGSVSTNLQSGNTQAATTDVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRYLTRNL

SEQ ID NO: 4 - protein, 735 aa, synthetic construct
MAADGYLPDWLEDTLSEGIRQWWKLKPGPPPPKPAERHQDDSRGLVLPGYKYLGPFNGLDKGEPVNEADVAALEHDKAYDRQLDSGDNPYLKYNHADAEFQERLKEDTSFGGNLGRAVFQAKKRVLEPLGLVEEPVKTAPGKKRPVEHSPAEPDSSSGTGKAGQQPARKRLNFGQTGDADSVPDPQPLGQPPAAPSGLGTNTMATGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSKTNAPSGTTTMSRLQFSQAGASDIRDQSRNWLPGPCYRQQRVSKTAADNNNSDYSWTGATKYHLNGRDSLVNPGPAMASHKDDEEKYFPQSGVLIFGKQDSGKTNVDIEKVMITDEEEIRTTNPVATEQYGSVSTNLQSGNTQAATTDVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRYLTRNL

SEQ ID NO: 5 - protein, 735 aa, synthetic construct
MAADGYLPDWLEDTLSEGIRQWWKLKPGPPPLKPAERHQDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPGKKRPVEHSPAEPDSSSGTGKAGQQPARKRLNFGQTGDADSVPDPQPLGQPPAAPSGLGTNTMAAGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSKTNAPSGTTTMSRLQFSQAGASDIRDQSRNWLPGPCYRQQRVSKTAADNNNSDYSWTGATKYHLNGRDSLVNPGPAMASHKDDEEKYFPQSGVLIFGKQDSGKTNVDIEKVMITDEEEIRTTNPVATEQYGSVSTNLQSGNTQAATTDVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRYLTRNL

SEQ ID NO: 6 - protein, 735 aa, synthetic construct
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLARAVFQAKKRVLEPLGLVEEPVKTAPGKKRPVEHSPAEPDSSSGTGKSGQQPARKRLNFGQTGDADSVPDPQPLGQPPAAPSGLGTNTMASGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSRTNTPSGTTTQSRLRFSQAGASDIRDQSRNWLPGPCYRQQRVSKTAADNNNSDYSWTGATKYHLNGRDSLVNPGTAMASHKDDEEKYFPQSGVLIFGKQDSEKTNVDIERVMITDEEEIRTTNPVATEQYGSVSTNLQSGNTQAATSDVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRYLTRNL

SEQ ID NO: 7 - protein, 735 aa, synthetic construct
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPGKKRPVEHSPVEPDSSSGTGKAGQQPARKRLNFGQTGDADSVPDPQPLGQPPAAPSGLGTNTMATGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLSFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSKTNAPSGTTTMSRLQFSQAGASDIRDQSRNWLPGPCYRQQRVSKTAADNNNSDYSWTGATKYHLNGRDSLVNPGPAMANHKDDEEKYFPQSGVLIFGKQGSNKTNVDIEKVMITDEEEIRTTNPVATEQYGSVSTNLQSGNTQAATSDVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRYLTRNL

SEQ ID NO: 8 - protein, 735 aa, synthetic construct
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPGKKRPVEHSPVESDSSSGTGKAGQQPARKRLNFGQTGDADSVPDPQPLGQPPAAPSGLGTNTMATGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLSFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSKTNAPSGTTTMSRLQFSQAGASDIRDQSRNWLPGPCYRQQRVSKTAADNNNSDYSWTGATKYHLNGRDSLVNPGPAMASHKDDEEKYFPQSGVLIFGKQGSNKTNVDIEKVMITDEEEIRTTNPVATEQYGSVSTNLQSGNTQAATSDVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRYLTRNL

SEQ ID NO: 9 - DNA, 2205 bp, unidentified
  source 1..2205: mol_type = genomic DNA; note = adeno-associated virus 2
atggcggcggatggctatctgccggattggctggaagataccctgagcgaaggcattcgccagtggtggaaactgaaaccgggcccgccgccgccgaaaccggcggaacgccataaagatgatagccgcggcctggtgctgccgggctataaatatctgggcccgtttaacggcctggataaaggcgaaccggtgaacgaagcggatgcggcggcgctggaacatgataaagcgtatgatcgccagctggatagcggcgataacccgtatctgaaatataaccatgcggatgcggaatttcaggaacgcctgaaagaagataccagctttggcggcaacctgggccgcgcggtgtttcaggcgaaaaaacgcgtgctggaaccgctgggcctggtggaagaaccggtgaaaaccgcgccgggcaaaaaacgcccggtggaacatagcccggtggaaccggatagcagcagcggcaccggcaaagcgggccagcagccggcgcgcaaacgcctgaactttggccagaccggcgatgcggatagcgtgccggatccgcagccgctgggccagccgccggcggcgccgagcggcctgggcaccaacaccatggcgaccggcagcggcgcgccgatggcggataacaacgaaggcgcggatggcgtgggcaacagcagcggcaactggcattgcgatagcacctggatgggcgatcgcgtgattaccaccagcacccgcacctgggcgctgccgacctataacaaccatctgtataaacagattagcagccagagcggcgcgagcaacgataaccattattttggctatagcaccccgtggggctattttgattttaaccgctttcattgccattttagcccgcgcgattggcagcgcctgattaacaacaactggggctttcgcccgaaacgcctgaactttaaactgtttaacattcaggtgaaagaagtgacccagaacgatggcaccaccaccattgcgaacaacctgaccagcaccgtgcaggtgtttaccgatagcgaatatcagctgccgtatgtgctgggcagcgcgcatcagggctgcctgccgccgtttccggcggatgtgtttatggtgccgcagtatggctatctgaccctgaacaacggcagccaggcggtgggccgcagcagcttttattgcctggaatattttccgagccagatgctgcgcaccggcaacaactttacctttagctatacctttgaagatgtgccgtttcatagcagctatgcgcatagccagagcctggatcgcctgatgaacccgctgattgatcagtatctgtattatctgagccgcaccaacaccccgagcggcaccaccacccagagccgcctgcagtttagccaggcgggcgcgagcgatattcgcgatcagagccgcaactggctgccgggcccgtgctatcgccagcagcgcgtgagcaaaaccagcgcggataacaacaacagcgaatatagctggaccggcgcgaccaaatatcatctgaacggccgcgatagcctggtgaacccgggcccggcgatggcgagccataaagatgatgaagaaaaattttttccgcagagcggcgtgctgatttttggcaaacagggcagcgaaaaaaccaacgtggatattgaaaaagtgatgattaccgatgaagaagaaattcgcaccaccaacccggtggcgaccgaacagtatggcagcgtgagcaccaacctgcagcgcggcaaccgccaggcggcgaccgcggatgtgaacacccagggcgtgctgccgggcatggtgtggcaggatcgcgatgtgtatctgcagggcccgatttgggcgaaaattccgcataccgatggccattttcatccgagcccgctgatgggcggctttggcctgaaacatccgccgccgcagattctgattaaaaacaccccggtgccggcgaacccgagcaccacctttagcgcggcgaaatttgcgagctttattacccagtatagcaccggccaggtgagcgtggaaattgaatgggaactgcagaaagaaaacagcaaacgctggaacccggaaattcagtataccagcaactataacaaaagcgtgaacgtggattttaccgtggataccaacggcgtgtatagcgaaccgcgcccgattggcacccgctatctgacccgcaacctg

SEQ ID NO: 10 - DNA, 2205 bp, synthetic construct
  source 1..2205: mol_type = other DNA
atggcggcggatggctatctgccggattggctggaagataccctgagcgaaggcattcgccagtggtggaaactgaaaccgggcccgccgccgccgaaaccggcggaacgccatcaggatgatagccgcggcctggtgctgccgggctataaatatctgggcccgtttaacggcctggataaaggcgaaccggtgagcgaagcggatgtggcggcgctggaacatgataaagcgtatgatcgccagctggatagcggcgataacccgtatctgaaatataaccatgcggatgcggaatttcaggaacgcctgaaagaagataccagctttggcggcaacctgggccgcgcggtgtttcaggcgaaaaaacgcgtgctggaaccgctgggcctggtggaagaaccggtgaaaaccgcgccgggcaaaaaacgcccggtggaacatagcccggcggaaccggatagcagcagcggcaccggcaaagcgggccagcagccggcgcgcaaacgcctgaactttggccagaccggcgatgcggatagcgtgccggatccgcagccgctgggccagccgccggcggcgccgagcggcctgggcaccaacaccatggcgaccggcagcggcgcgccgatggcggataacaacgaaggcgcggatggcgtgggcaacagcagcggcaactggcattgcgatagcacctggatgggcgatcgcgtgattaccaccagcacccgcacctgggcgctgccgacctataacaaccatctgtataaacagattagcagccagagcggcgcgagcaacgataaccattattttggctatagcaccccgtggggctattttgattttaaccgctttcattgccattttagcccgcgcgattggcagcgcctgattaacaacaactggggctttcgcccgaaacgcctgaactttaaactgtttaacattcaggtgaaagaagtgacccagaacgatggcaccaccaccattgcgaacaacctgaccagcaccgtgcaggtgtttaccgatagcgaatatcagctgccgtatgtgctgggcagcgcgcatcagggctgcctgccgccgtttccggcggatgtgtttatggtgccgcagtatggctatctgaccctgaacaacggcagccaggcggtgggccgcagcagcttttattgcctggaatattttccgagccagatgctgcgcaccggcaacaactttacctttagctatacctttgaagatgtgccgtttcatagcagctatgcgcatagccagagcctggatcgcctgatgaacccgctgattgatcagtatctgtattatctgagcaaaaccaacgcgccgagcggcaccaccaccatgagccgcctgcagtttagccaggcgggcgcgagcgatattcgcgatcagagccgcaactggctgccgggcccgtgctatcgccagcagcgcgtgagcaaaaccgcggcggataacaacaacagcgattatagctggaccggcgcgaccaaatatcatctgaacggccgcgatagcctggtgaacccgggcccggcgatggcgagccataaagatgatgaagaaaaatattttccgcagagcggcgtgctgatttttggcaaacaggatagcggcaaaaccaacgtggatattgaaaaagtgatgattaccgatgaagaagaaattcgcaccaccaacccggtggcgaccgaacagtatggcagcgtgagcaccaacctgcagagcggcaacacccaggcggcgaccaccgatgtgaacacccagggcgtgctgccgggcatggtgtggcaggatcgcgatgtgtatctgcagggcccgatttgggcgaaaattccgcataccgatggccattttcatccgagcccgctgatgggcggctttggcctgaaacatccgccgccgcagattctgattaaaaacaccccggtgccggcgaacccgagcaccacctttagcgcggcgaaatttgcgagctttattacccagtatagcaccggccaggtgagcgtggaaattgaatgggaactgcagaaagaaaacagcaaacgctggaacccggaaattcagtataccagcaactataacaaaagcgtgaacgtggattttaccgtggataccaacggcgtgtatagcgaaccgcgcccgattggcacccgctatctgacccgcaacctg

SEQ ID NO: 11 - DNA, 2205 bp, synthetic construct
  source 1..2205: mol_type = other DNA
atggcggcggatggctatctgccggattggctggaagataccctgagcgaaggcattcgccagtggtggaaactgaaaccgggcccgccgccgccgaaaccggcggaacgccatcaggatgatagccgcggcctggtgctgccgggctataaatatctgggcccgtttaacggcctggataaaggcgaaccggtgaacgaagcggatgcggcggcgctggaacatgataaagcgtatgatcgccagctggatagcggcgataacccgtatctgaaatataaccatgcggatgcggaatttcaggaacgcctgaaagaagataccagctttggcggcaacctgggccgcgcggtgtttcaggcgaaaaaacgcgtgctggaaccgctgggcctggtggaagaaccggtgaaaaccgcgccgggcaaaaaacgcccggtggaacattttccggcggaaccggatagcagcagcggcaccggcaaagcgggccagcagccggcgcgcaaacgcctgaactttggccagaccggcgatgcggatagcgtgccggatccgcagccgctgggccagccgccggcggcgccgagcggcctgggcaccaacaccatggcgaccggcagcggcgcgccgatggcggataacaacgaaggcgcggatggcgtgggcaacagcagcggcaactggcattgcgatagcacctggatgggcgatcgcgtgattaccaccagcacccgcacctgggcgctgccgacctataacaaccatctgtataaacagattagcagccagagcggcgcgagcaacgataaccattattttggctatagcaccccgtggggctattttgattttaaccgctttcattgccattttagcccgcgcgattggcagcgcctgattaacaacaactggggctttcgcccgaaacgcctgaactttaaactgtttaacattcaggtgaaagaagtgacccagaacgatggcaccaccaccattgcgaacaacctgaccagcaccgtgcaggtgtttaccgatagcgaatatcagctgccgtatgtgctgggcagcgcgcatcagggctgcctgccgccgtttccggcggatgtgtttatggtgccgcagtatggctatctgaccctgaacaacggcagccaggcggtgggccgcagcagcttttattgcctggaatattttccgagccagatgctgcgcaccggcaacaactttacctttagctatacctttgaagatgtgccgtttcatagcagctatgcgcatagccagagcctggatcgcctgatgaacccgctgattgatcagtatctgtattatctgagcaaaaccaacgcgccgagcggcaccaccaccatgagccgcctgcagtttagccaggcgggcgcgagcgatattcgcgatcagagccgcaactggctgccgggcccgtgctatcgccagcagcgcgtgagcaaaaccgcggcggataacaacaacagcgattatagctggaccggcgcgaccaaatatcatctgaacggccgcgatagcctggtgaacccgggcccggcgatggcgagccataaagatgatgaagaaaaatattttccgcagagcggcgtgctgatttttggcaaacaggatagcggcaaaaccaacgtggatattgaaaaagtgatgattaccgatgaagaagaaattcgcaccaccaacccggtggcgaccgaacagtatggcagcgtgagcaccaacctgcagagcggcaacacccaggcggcgaccaccgatgtgaacacccagggcgtgctgccgggcatggtgtggcaggatcgcgatgtgtatctgcagggcccgatttgggcgaaaattccgcataccgatggccattttcatccgagcccgctgatgggcggctttggcctgaaacatccgccgccgcagattctgattaaaaacaccccggtgccggcgaacccgagcaccacctttagcgcggcgaaatttgcgagctttattacccagtatagcaccggccaggtgagcgtggaaattgaatgggaactgcagaaagaaaacagcaaacgctggaacccggaaattcagtataccagcaactataacaaaagcgtgaacgtggattttaccgtggataccaacggcgtgtatagcgaaccgcgcccgattggcacccgctatctgacccgcaacctg

SEQ ID NO: 12 - DNA, 2205 bp, synthetic construct
  source 1..2205: mol_type = other DNA
atggcggcggatggctatctgccggattggctggaagataccctgagcgaaggcattcgccagtggtggaaactgaaaccgggcccgccgccgccgaaaccggcggaacgccatcaggatgatagccgcggcctggtgctgccgggctataaatatctgggcccgtttaacggcctggataaaggcgaaccggtgaacgaagcggatgtggcggcgctggaacatgataaagcgtatgatcgccagctggatagcggcgataacccgtatctgaaatataaccatgcggatgcggaatttcaggaacgcctgaaagaagataccagctttggcggcaacctgggccgcgcggtgtttcaggcgaaaaaacgcgtgctggaaccgctgggcctggtggaagaaccggtgaaaaccgcgccgggcaaaaaacgcccggtggaacatagcccggcggaaccggatagcagcagcggcaccggcaaagcgggccagcagccggcgcgcaaacgcctgaactttggccagaccggcgatgcggatagcgtgccggatccgcagccgctgggccagccgccggcggcgccgagcggcctgggcaccaacaccatggcgaccggcagcggcgcgccgatggcggataacaacgaaggcgcggatggcgtgggcaacagcagcggcaactggcattgcgatagcacctggatgggcgatcgcgtgattaccaccagcacccgcacctgggcgctgccgacctataacaaccatctgtataaacagattagcagccagagcggcgcgagcaacgataaccattattttggctatagcaccccgtggggctattttgattttaaccgctttcattgccattttagcccgcgcgattggcagcgcctgattaacaacaactggggctttcgcccgaaacgcctgaactttaaactgtttaacattcaggtgaaagaagtgacccagaacgatggcaccaccaccattgcgaacaacctgaccagcaccgtgcaggtgtttaccgatagcgaatatcagctgccgtatgtgctgggcagcgcgcatcagggctgcctgccgccgtttccggcggatgtgtttatggtgccgcagtatggctatctgaccctgaacaacggcagccaggcggtgggccgcagcagcttttattgcctggaatattttccgagccagatgctgcgcaccggcaacaactttacctttagctatacctttgaagatgtgccgtttcatagcagctatgcgcatagccagagcctggatcgcctgatgaacccgctgattgatcagtatctgtattatctgagcaaaaccaacgcgccgagcggcaccaccaccatgagccgcctgcagtttagccaggcgggcgcgagcgatattcgcgatcagagccgcaactggctgccgggcccgtgctatcgccagcagcgcgtgagcaaaaccgcggcggataacaacaacagcgattatagctggaccggcgcgaccaaatatcatctgaacggccgcgatagcctggtgaacccgggcccggcgatggcgagccataaagatgatgaagaaaaatattttccgcagagcggcgtgctgatttttggcaaacaggatagcggcaaaaccaacgtggatattgaaaaagtgatgattaccgatgaagaagaaattcgcaccaccaacccggtggcgaccgaacagtatggcagcgtgagcaccaacctgcagagcggcaacacccaggcggcgaccaccgatgtgaacacccagggcgtgctgccgggcatggtgtggcaggatcgcgatgtgtatctgcagggcccgatttgggcgaaaattccgcataccgatggccattttcatccgagcccgctgatgggcggctttggcctgaaacatccgccgccgcagattctgattaaaaacaccccggtgccggcgaacccgagcaccacctttagcgcggcgaaatttgcgagctttattacccagtatagcaccggccaggtgagcgtggaaattgaatgggaactgcagaaagaaaacagcaaacgctggaacccggaaattcagtataccagcaactataacaaaagcgtgaacgtggattttaccgtggataccaacggcgtgtatagcgaaccgcgcccgattggcacccgctatctgacccgcaacctg

SEQ ID NO: 13 - DNA, 2205 bp, synthetic construct
  source 1..2205: mol_type = other DNA
atggcggcggatggctatctgccggattggctggaagataccctgagcgaaggcattcgccagtggtggaaactgaaaccgggcccgccgccgctgaaaccggcggaacgccatcaggatgatagccgcggcctggtgctgccgggctataaatatctgggcccgtttaacggcctggataaaggcgaaccggtgaacgaagcggatgcggcggcgctggaacatgataaagcgtatgatcgccagctggatagcggcgataacccgtatctgaaatataaccatgcggatgcggaatttcaggaacgcctgaaagaagataccagctttggcggcaacctgggccgcgcggtgtttcaggcgaaaaaacgcgtgctggaaccgctgggcctggtggaagaaccggtgaaaaccgcgccgggcaaaaaacgcccggtggaacatagcccggcggaaccggatagcagcagcggcaccggcaaagcgggccagcagccggcgcgcaaacgcctgaactttggccagaccggcgatgcggatagcgtgccggatccgcagccgctgggccagccgccggcggcgccgagcggcctgggcaccaacaccatggcggcgggcagcggcgcgccgatggcggataacaacgaaggcgcggatggcgtgggcaacagcagcggcaactggcattgcgatagcacctggatgggcgatcgcgtgattaccaccagcacccgcacctgggcgctgccgacctataacaaccatctgtataaacagattagcagccagagcggcgcgagcaacgataaccattattttggctatagcaccccgtggggctattttgattttaaccgctttcattgccattttagcccgcgcgattggcagcgcctgattaacaacaactggggctttcgcccgaaacgcctgaactttaaactgtttaacattcaggtgaaagaagtgacccagaacgatggcaccaccaccattgcgaacaacctgaccagcaccgtgcaggtgtttaccgatagcgaatatcagctgccgtatgtgctgggcagcgcgcatcagggctgcctgccgccgtttccggcggatgtgtttatggtgccgcagtatggctatctgaccctgaacaacggcagccaggcggtgggccgcagcagcttttattgcctggaatattttccgagccagatgctgcgcaccggcaacaactttacctttagctatacctttgaagatgtgccgtttcatagcagctatgcgcatagccagagcctggatcgcctgatgaacccgctgattgatcagtatctgtattatctgagcaaaaccaacgcgccgagcggcaccaccaccatgagccgcctgcagtttagccaggcgggcgcgagcgatattcgcgatcagagccgcaactggctgccgggcccgtgctatcgccagcagcgcgtgagcaaaaccgcggcggataacaacaacagcgattatagctggaccggcgcgaccaaatatcatctgaacggccgcgatagcctggtgaacccgggcccggcgatggcgagccataaagatgatgaagaaaaatattttccgcagagcggcgtgctgatttttggcaaacaggatagcggcaaaaccaacgtggatattgaaaaagtgatgattaccgatgaagaagaaattcgcaccaccaacccggtggcgaccgaacagtatggcagcgtgagcaccaacctgcagagcggcaacacccaggcggcgaccaccgatgtgaacacccagggcgtgctgccgggcatggtgtggcaggatcgcgatgtgtatctgcagggcccgatttgggcgaaaattccgcataccgatggccattttcatccgagcccgctgatgggcggctttggcctgaaacatccgccgccgcagattctgattaaaaacaccccggtgccggcgaacccgagcaccacctttagcgcggcgaaatttgcgagctttattacccagtatagcaccggccaggtgagcgtggaaattgaatgggaactgcagaaagaaaacagcaaacgctggaacccggaaattcagtataccagcaactataacaaaagcgtgaacgtggattttaccgtggataccaacggcgtgtatagcgaaccgcgcccgattggcacccgctatctgacccgcaacctg

SEQ ID NO: 14 - DNA, 2205 bp, synthetic construct
  source 1..2205: mol_type = other DNA
atggcggcggatggctatctgccggattggctggaagataccctgagcgaaggcattcgccagtggtggaaactgaaaccgggcccgccgccgccgaaaccggcggaacgccataaagatgatagccgcggcctggtgctgccgggctataaatatctgggcccgtttaacggcctggataaaggcgaaccggtgaacgaagcggatgcggcggcgctggaacatgataaagcgtatgatcgccagctggatagcggcgataacccgtatctgaaatataaccatgcggatgcggaatttcaggaacgcctgaaagaagataccagctttggcggcaacctggcgcgcgcggtgtttcaggcgaaaaaacgcgtgctggaaccgctgggcctggtggaagaaccggtgaaaaccgcgccgggcaaaaaacgcccggtggaacatagcccggcggaaccggatagcagcagcggcaccggcaaaagcggccagcagccggcgcgcaaacgcctgaactttggccagaccggcgatgcggatagcgtgccggatccgcagccgctgggccagccgccggcggcgccgagcggcctgggcaccaacaccatggcgagcggcagcggcgcgccgatggcggataacaacgaaggcgcggatggcgtgggcaacagcagcggcaactggcattgcgatagcacctggatgggcgatcgcgtgattaccaccagcacccgcacctgggcgctgccgacctataacaaccatctgtataaacagattagcagccagagcggcgcgagcaacgataaccattattttggctatagcaccccgtggggctattttgattttaaccgctttcattgccattttagcccgcgcgattggcagcgcctgattaacaacaactggggctttcgcccgaaacgcctgaactttaaactgtttaacattcaggtgaaagaagtgacccagaacgatggcaccaccaccattgcgaacaacctgaccagcaccgtgcaggtgtttaccgatagcgaatatcagctgccgtatgtgctgggcagcgcgcatcagggctgcctgccgccgtttccggcggatgtgtttatggtgccgcagtatggctatctgaccctgaacaacggcagccaggcggtgggccgcagcagcttttattgcctggaatattttccgagccagatgctgcgcaccggcaacaactttacctttagctatacctttgaagatgtgccgtttcatagcagctatgcgcatagccagagcctggatcgcctgatgaacccgctgattgatcagtatctgtattatctgagccgcaccaacaccccgagcggcaccaccacccagagccgcctgcgctttagccaggcgggcgcgagcgatattcgcgatcagagccgcaactggctgccgggcccgtgctatcgccagcagcgcgtgagcaaaaccgcggcggataacaacaacagcgattatagctggaccggcgcgaccaaatatcatctgaacggccgcgatagcctggtgaacccgggcaccgcgatggcgagccataaagatgatgaagaaaaatattttccgcagagcggcgtgctgatttttggcaaacaggatagcgaaaaaaccaacgtggatattgaacgcgtgatgattaccgatgaagaagaaattcgcaccaccaacccggtggcgaccgaacagtatggcagcgtgagcaccaacctgcagagcggcaacacccaggcggcgaccagcgatgtgaacacccagggcgtgctgccgggcatggtgtggcaggatcgcgatgtgtatctgcagggcccgatttgggcgaaaattccgcataccgatggccattttcatccgagcccgctgatgggcggctttggcctgaaacatccgccgccgcagattctgattaaaaacaccccggtgccggcgaacccgagcaccacctttagcgcggcgaaatttgcgagctttattacccagtatagcaccggccaggtgagcgtggaaattgaatgggaactgcagaaagaaaacagcaaacgctggaacccggaaattcagtataccagcaactataacaaaagcgtgaacgtggattttaccgtggataccaacggcgtgtatagcgaaccgcgcccgattggcacccgctatctgacccgcaacctg

SEQ ID NO: 15 - DNA, 2205 bp, synthetic construct
  source 1..2205: mol_type = other DNA
atggcggcggatggctatctgccggattggctggaagataccctgagcgaaggcattcgccagtggtggaaactgaaaccgggcccgccgccgccgaaaccggcggaacgccataaagatgatagccgcggcctggtgctgccgggctataaatatctgggcccgtttaacggcctggataaaggcgaaccggtgaacgaagcggatgcggcggcgctggaacatgataaagcgtatgatcgccagctggatagcggcgataacccgtatctgaaatataaccatgcggatgcggaatttcaggaacgcctgaaagaagataccagctttggcggcaacctgggccgcgcggtgtttcaggcgaaaaaacgcgtgctggaaccgctgggcctggtggaagaaccggtgaaaaccgcgccgggcaaaaaacgcccggtggaacatagcccggtggaaccggatagcagcagcggcaccggcaaagcgggccagcagccggcgcgcaaacgcctgaactttggccagaccggcgatgcggatagcgtgccggatccgcagccgctgggccagccgccggcggcgccgagcggcctgggcaccaacaccatggcgaccggcagcggcgcgccgatggcggataacaacgaaggcgcggatggcgtgggcaacagcagcggcaactggcattgcgatagcacctggatgggcgatcgcgtgattaccaccagcacccgcacctgggcgctgccgacctataacaaccatctgtataaacagattagcagccagagcggcgcgagcaacgataaccattattttggctatagcaccccgtggggctattttgattttaaccgctttcattgccattttagcccgcgcgattggcagcgcctgattaacaacaactggggctttcgcccgaaacgcctgagctttaaactgtttaacattcaggtgaaagaagtgacccagaacgatggcaccaccaccattgcgaacaacctgaccagcaccgtgcaggtgtttaccgatagcgaatatcagctgccgtatgtgctgggcagcgcgcatcagggctgcctgccgccgtttccggcggatgtgtttatggtgccgcagtatggctatctgaccctgaacaacggcagccaggcggtgggccgcagcagcttttattgcctggaatattttccgagccagatgctgcgcaccggcaacaactttacctttagctatacctttgaagatgtgccgtttcatagcagctatgcgcatagccagagcctggatcgcctgatgaacccgctgattgatcagtatctgtattatctgagcaaaaccaacgcgccgagcggcaccaccaccatgagccgcctgcagtttagccaggcgggcgcgagcgatattcgcgatcagagccgcaactggctgccgggcccgtgctatcgccagcagcgcgtgagcaaaaccgcggcggataacaacaacagcgattatagctggaccggcgcgaccaaatatcatctgaacggccgcgatagcctggtgaacccgggcccggcgatggcgaaccataaagatgatgaagaaaaatattttccgcagagcggcgtgctgatttttggcaaacagggcagcaacaaaaccaacgtggatattgaaaaagtgatgattaccgatgaagaagaaattcgcaccaccaacccggtggcgaccgaacagtatggcagcgtgagcaccaacctgcagagcggcaacacccaggcggcgaccagcgatgtgaacacccagggcgtgctgccgggcatggtgtggcaggatcgcgatgtgtatctgcagggcccgatttgggcgaaaattccgcataccgatggccattttcatccgagcccgctgatgggcggctttggcctgaaacatccgccgccgcagattctgattaaaaacaccccggtgccggcgaacccgagcaccacctttagcgcggcgaaatttgcgagctttattacccagtatagcaccggccaggtgagcgtggaaattgaatgggaactgcagaaagaaaacagcaaacgctggaacccggaaattcagtataccagcaactataacaaaagcgtgaacgtggattttaccgtggataccaacggcgtgtatagcgaaccgcgcccgattggcacccgctatctgacccgcaacctg

SEQ ID NO: 16 - DNA, 2205 bp, synthetic construct
  source 1..2205: mol_type = other DNA
atggcggcggatggctatctgccggattggctggaagataccctgagcgaaggcattcgccagtggtggaaactgaaaccgggcccgccgccgccgaaaccggcggaacgccataaagatgatagccgcggcctggtgctgccgggctataaatatctgggcccgtttaacggcctggataaaggcgaaccggtgaacgaagcggatgcggcggcgctggaacatgataaagcgtatgatcgccagctggatagcggcgataacccgtatctgaaatataaccatgcggatgcggaatttcaggaacgcctgaaagaagataccagctttggcggcaacctgggccgcgcggtgtttcaggcgaaaaaacgcgtgctggaaccgctgggcctggtggaagaaccggtgaaaaccgcgccgggcaaaaaacgcccggtggaacatagcccggtggaaagcgatagcagcagcggcaccggcaaagcgggccagcagccggcgcgcaaacgcctgaactttggccagaccggcgatgcggatagcgtgccggatccgcagccgctgggccagccgccggcggcgccgagcggcctgggcaccaacaccatggcgaccggcagcggcgcgccgatggcggataacaacgaaggcgcggatggcgtgggcaacagcagcggcaactggcattgcgatagcacctggatgggcgatcgcgtgattaccaccagcacccgcacctgggcgctgccgacctataacaaccatctgtataaacagattagcagccagagcggcgcgagcaacgataaccattattttggctatagcaccccgtggggctattttgattttaaccgctttcattgccattttagcccgcgcgattggcagcgcctgattaacaacaactggggctttcgcccgaaacgcctgagctttaaactgtttaacattcaggtgaaagaagtgacccagaacgatggcaccaccaccattgcgaacaacctgaccagcaccgtgcaggtgtttaccgatagcgaatatcagctgccgtatgtgctgggcagcgcgcatcagggctgcctgccgccgtttccggcggatgtgtttatggtgccgcagtatggctatctgaccctgaacaacggcagccaggcggtgggccgcagcagcttttattgcctggaatattttccgagccagatgctgcgcaccggcaacaactttacctttagctatacctttgaagatgtgccgtttcatagcagctatgcgcatagccagagcctggatcgcctgatgaacccgctgattgatcagtatctgtattatctgagcaaaaccaacgcgccgagcggcaccaccaccatgagccgcctgcagtttagccaggcgggcgcgagcgatattcgcgatcagagccgcaactggctgccgggcccgtgctatcgccagcagcgcgtgagcaaaaccgcggcggataacaacaacagcgattatagctggaccggcgcgaccaaatatcatctgaacggccgcgatagcctggtgaacccgggcccggcgatggcgagccataaagatgatgaagaaaaatattttccgcagagcggcgtgctgatttttggcaaacagggcagcaacaaaaccaacgtggatattgaaaaagtgatgattaccgatgaagaagaaattcgcaccaccaacccggtggcgaccgaacagtatggcagcgtgagcaccaacctgcagagcggcaacacccaggcggcgaccagcgatgtgaacacccagggcgtgctgccgggcatggtgtggcaggatcgcgatgtgtatctgcagggcccgatttgggcgaaaattccgcataccgatggccattttcatccgagcccgctgatgggcggctttggcctgaaacatccgccgccgcagattctgattaaaaacaccccggtgccggcgaacccgagcaccacctttagcgcggcgaaatttgcgagctttattacccagtatagcaccggccaggtgagcgtggaaattgaatgggaactgcagaaagaaaacagcaaacgctggaacccggaaattcagtataccagcaactataacaaaagcgtgaacgtggattttaccgtggataccaacggcgtgtatagcgaaccgcgcccgattggcacccgctatctgacccgcaacctg

(19) United States
(12) Patent Application Publication
(10) Pub. No.: US 2025/0387511 A1
(43) Pub. Date: Dec. 25, 2025

(54) AAV2 VARIANTS AND USES THEREOF

(71) Applicant: University of Massachusetts, Westborough, MA (US)

(72) Inventors: Guangping Gao, Worcester, MA (US); Phillip Tai, Worcester, MA (US); Sophia Yonglin Liu, Worcester, MA (US); Thomas Bovard Leland, Northborough, MA (US)

(73) Assignee: University of Massachusetts, Westborough, MA (US)

(21) Appl. No.: 19/250,243

(22) Filed: Jun. 26, 2025

Related U.S. Application Data
(63) Continuation of application No. PCT/US2024/024727, filed on Apr. 16, 2024.
(60) Provisional application No. 63/496,428, filed on Apr. 17, 2023.
(60) Provisional application No. 63/507,138, filed on Jun. 9, 2023.

(51) Int. Cl. A61K 48/00; A61K 9/00; C07K 14/005; C12N 15/86

(52) U.S. Cl. CPC A61K 48/0041; A61K 9/0048; A61K 48/0075; C07K 14/005; C12N 15/86; C12N 2750/14122; C12N 2750/14143

(57) ABSTRACT

Aspects of the disclosure relate to compositions and methods for delivering a transgene (e.g., a transgene encoding one or more gene products) to a target cell (e.g., a brain cell). The disclosure is based, in part, on adeno-associated virus (AAV) capsid proteins comprising one or more amino acid substitutions, and methods of using the same for delivery of a transgene to tissues and cells of the central nervous system (e.g., brain cells).

### RELATED APPLICATIONS

This application claims the benefit under 35 U.S.C. § 365 (c) and § 120 and is a continuation of International Patent Application Number PCT/US2024/024727, filed Apr. 16, 2024, titled “AAV2 VARIANTS AND USES THEREOF”, which claims priority under 35 U.S.C. § 119 (e) to U.S. provisional patent application 63/496,428, filed on Apr. 17, 2023 and U.S. Provisional Application No. 63/507,138, filed on Jun. 9, 2023, the entire contents of each of which are incorporated by reference herein.

### REFERENCE TO AN ELECTRONIC SEQUENCE LISTING

The contents of the electronic sequence listing (U012070186US02-SEQ-KZM.xml; Size: 38,694 bytes; and Date of Creation: Jun. 24, 2025) is herein incorporated by reference in its entirety.

### BACKGROUND OF INVENTION

Recombinant AAV adeno-associated viruses (rAAVs) are capable of driving stable and sustained transgene expression in target tissues without notable toxicity and host immunogenicity. Thus, rAAVs are promising delivery vehicles for long-term therapeutic gene expression. However, low transduction efficiency and restricted tissue tropisms by currently available rAAV vectors can limit their application as feasible and efficacious therapies. Furthermore, inability to cross the blood-brain barrier limits the applicability of rAAVs as therapeutic systems for disorders affecting the central nervous system. Additionally, faithful clinical translation of leading therapeutic AAV serotypes derived from non-human tissues is a concern. Accordingly, a need remains for new AAV vectors for gene delivery, especially those that have the capability of traversing the blood-brain barrier and penetrating deep brain structures.

### SUMMARY OF INVENTION

Aspects of the disclosure relate to novel compositions and methods for delivering a transgene (e.g., a transgene encoding one or more gene products) to a target cell (e.g., a cell of the central nervous system (e.g., brain cells)). The disclosure is based, in part, on adeno-associated virus (AAV) capsid protein variants characterized by tropisms for certain cell types (e.g., CNS and brain cells such as neurons, astrocytes, oligodendrocytes, Muller glial cells, Schwann cells, enteric glial cells, or microglial cells). According to some embodiments, variants of AAV2 capsid protein have been identified and are disclosed herein that possess useful tissue targeting properties. In some embodiments, recombinant AAVs (rAAVs) comprising the capsid protein variants can cross the blood-brain barrier of subjects administered (e.g., transduced with) said rAAVs. In some embodiments, rAAVs comprising the capsid protein variants transduce deep brain structures (e.g., the deep cerebellar cortex, hippocampus, motor cortex, deep midbrain, substantia nigra, dentate gyrus, ventral lateral nucleus, or ventral anterior nucleus) more readily than rAAVs comprising wild-type capsid proteins. Methods of delivering an rAAV comprising the AAV capsid protein variants are also described by the disclosure.

Accordingly, in some aspects, the disclosure provides a method for delivering a transgene to central nervous system (CNS) cell of a subject, the method comprising administering to the subject a recombinant adeno-associated virus (rAAV) comprising an isolated nucleic acid comprising a transgene encoding one or more gene products flanked by adeno-associated virus (AAV) inverted terminal repeats (ITRs); and an adeno-associated virus (AAV) capsid protein, wherein the capsid protein comprises one or more amino acid substitutions corresponding to position P32, K39, N66, A70, G115, S149, V151, P153, A126, T205, N312, R447, T450, Q457, Q461, S492, E499, P521, S525, F533, G546, E548, K556, R585, R588, and/or A593 with reference to amino acid position numbering of a wild-type AAV2 capsid protein (e.g., SEQ ID NO: 1). In some embodiments, the capsid protein has at least 80%, 85%, 90%, 95%, 97%, or 98% sequence identity to the amino acid sequence set forth in SEQ ID NO: 1 but is not 100% identical to the amino acid sequence set forth in SEQ ID NO: 1.

In some embodiments, the capsid protein comprises an amino acid substitution corresponding to P32L, K39Q, N66S, A70V, G115A, S149F, V151A, P153S, A162S, T205A, T205S, N312S, R447K, T450A, Q457M, Q461R, S492A, E499D, P521T, S525N, F533Y, G546D, E548G, E548N, K556R, R585S, R588T, A593T, and/or A593S with reference to amino acid position numbering of a wild-type AAV2 capsid protein (e.g., SEQ ID NO: 1).

In some embodiments, the capsid protein comprises amino acid substitutions corresponding to positions K39, N66, A70, V151, R447, T450, Q457, S492, E499, F533, G546, E548, R585, R588, and A593 with reference to amino acid position numbering of a wild-type AAV2 capsid protein (e.g., SEQ ID NO: 1).

In some embodiments, the capsid protein comprises amino acid substitutions corresponding to K39Q, N66S, A70V, V151A, R447K, T450A, Q457M, S492A, E499D, F533Y, G546D, E548G, R585S, R588T, and A593T with reference to amino acid position numbering of a wild-type AAV2 capsid protein (e.g., SEQ ID NO: 1). In some embodiments, the capsid protein comprises the amino acid sequence set forth in SEQ ID NO: 2.

In some embodiments, the capsid protein comprises amino acid substitutions corresponding to positions K39, S149, V151, R447, T450, Q457, S492, E499, F533, G546, E548, R585, R588, and A593 with reference to amino acid position numbering of a wild-type AAV2 capsid protein (e.g., SEQ ID NO: 1). In some embodiments, the capsid protein comprises amino acid substitutions corresponding to K39Q, S149F, V151A, R447K, T450A, Q457M, S492A, E499D, F533Y, G546D, E548G, R585S, R588T, and A593T with reference to amino acid position numbering of a wild-type AAV2 capsid protein (e.g., SEQ ID NO: 1). In some embodiments, the capsid protein comprises the amino acid sequence set forth in SEQ ID NO: 3.

In some embodiments, the capsid protein comprises amino acid substitutions corresponding to positions K39, A70, V151, R447, T450, Q457, S492, E499, F533, G546, E548, R585, R588, and A593 with reference to amino acid position numbering of a wild-type AAV2 capsid protein (e.g., SEQ ID NO: 1). In some embodiments, the capsid protein comprises amino acid substitutions corresponding to K39Q, A70V, V151A, R447K, T450A, Q457M, S492A, E499D, F533Y, G546D, E548G, R585S, R588T, and A593T with reference to amino acid position numbering of a wild-type AAV2 capsid protein (e.g., SEQ ID NO: 1). In some embodiments, the capsid protein comprises the amino acid sequence set forth in SEQ ID NO: 4.

In some embodiments, the capsid protein comprises amino acid substitutions corresponding to positions P32, K39, V151, T205, R447, T450, Q457, S492, E499, F533, G546, E548, R585, R588, and A593 with reference to amino acid position numbering of a wild-type AAV2 capsid protein (e.g., SEQ ID NO: 1). In some embodiments, the capsid protein comprises amino acid substitutions corresponding to P32L, K39Q, V151A, T205A, R447K, T450A, Q457M, S492A, E499D, F533Y, G546D, E548G, R585S, R588T, and A593T with reference to amino acid position numbering of a wild-type AAV2 capsid protein (e.g., SEQ ID NO: 1). In some embodiments, the capsid protein comprises the amino acid sequence set forth in SEQ ID NO: 5.

In some embodiments, the capsid protein comprises amino acid substitutions corresponding to positions G115, V151, A162, T205, Q461, S492, E499, P521, F533, G546, K556, R585, R588, and A593 with reference to amino acid position numbering of a wild-type AAV2 capsid protein (e.g., SEQ ID NO: 1). In some embodiments, the capsid protein comprises amino acid substitutions corresponding to G115A, V151A, A162S, T205S, Q461R, S492A, E499D, P521T, F533Y, G546D, K566R, R585S, R588T, and A593S with reference to amino acid position numbering of a wild-type AAV2 capsid protein (e.g., SEQ ID NO: 1). In some embodiments, the capsid protein comprises the amino acid sequence set forth in SEQ ID NO: 6.

In some embodiments, the capsid protein comprises amino acid substitutions corresponding to positions N312, R447, T450, Q457, S492, E499, S525, F533, E548, R585, R588, and A593 with reference to amino acid position numbering of a wild-type AAV2 capsid protein (e.g., SEQ ID NO: 1). In some embodiments, the capsid protein comprises amino acid substitutions corresponding to N312S, R447K, T450A, Q457M, S492A, E499D, S525N, F533Y, E548N, R585S, R588T, and A593S with reference to amino acid position numbering of a wild-type AAV2 capsid protein (e.g., SEQ ID NO: 1). In some embodiments, the capsid protein comprises the amino acid sequence set forth in SEQ ID NO: 7.

In some embodiments, the capsid protein comprises amino acid substitutions corresponding to positions P153, N312, R447, T450, Q457, S492, E499, F533, E548, R585, R588, and A593 with reference to amino acid position numbering of a wild-type AAV2 capsid protein (e.g., SEQ ID NO: 1). In some embodiments, the capsid protein comprises amino acid substitutions corresponding to P153S, N312S, R447K, T450A, Q457M, S492A, E499D, F533Y, E548N, R585S, R588T, and A593S with reference to amino acid position numbering of a wild-type AAV2 capsid protein (e.g., SEQ ID NO: 1). In some embodiments, the capsid protein comprises the amino acid sequence set forth in SEQ ID NO: 8.

In some aspects, the disclosure provides a method for delivering a transgene to a cell of the central nervous system (CNS) in a subject, the method comprising administering to the subject a recombinant adeno-associated virus (rAAV) comprising an isolated nucleic acid comprising a transgene encoding one or more gene products flanked by adeno associated virus (AAV) inverted terminal repeats (ITRs); and an AAV capsid protein comprising the amino acid sequence set forth in any one of SEQ ID NOs: 2-8.

In some embodiments, administration comprises intrahippocampal injection, intraparenchymal injection, intravenous (IV) injection, or intracerebroventricular (ICV) injection. In some embodiments, administration comprises intracranial injection.

In some embodiments, administration of a rAAV comprising an AAV capsid protein as described herein (e.g., an AAV capsid protein comprising the amino acid sequence set forth in any one of SEQ ID NOs: 2-8) results in the rAAV crossing the blood-brain barrier.

In some embodiments, a cell of the CNS is present in the corpus callosum, cornu ammonis, fimbria, polymorph layer of the dentate gyrus, and granule cell layer of the dentate gyrus of the hippocampus of a subject. In some embodiments, a cell of the CNS of a subject is a neuron, astrocyte, oligodendrocyte, Muller glial cell, Schwann cell, enteric glial cell, or microglial cell.

In some embodiments, a subject is a mammal. In some embodiments, a mammal is a human.

In some embodiments, a nucleic acid sequence encoding one or more gene products is operably linked to a promoter. In some embodiments, a promoter is a CNS-specific promoter. In some embodiments, a CNS-specific promoter is a glial fibrillary acidic protein (GFAP) promoter, gfaABC1D promoter, gfa28/gfaABD promoter, ALDH1L1 promoter, gfa2 promoter, gfa2(B3) promoter, Mbp promoter, MAG promoter, Cbh promoter, F4/80 promoter, CD68 promoter, CD11B promoter, RLBP1 promoter, ProB2 promoter, Mpz promoter, or Cnp promoter.

In some embodiments, one or more gene products comprise a protein or an inhibitory nucleic acid. In some embodiments, one or more gene products comprise a therapeutic peptide, polypeptide, siRNA, microRNA, and/or RNA aptamer.

In some embodiments, an rAAV as described herein crosses the blood-brain barrier of a subject administration.

In some aspects, the disclosure provides a recombinant expression vector comprising a nucleic acid encoding a polypeptide comprising the sequence as set forth in any one of SEQ ID NOs: 2-8.

In some aspects, the disclosure provides a recombinant expression vector comprising a nucleic acid comprising the sequence as set forth in any one of SEQ ID NOs: 10-16. In some aspects, the disclosure provides an isolated AAV capsid protein comprising an amino acid sequence having a sequence as set forth in any one of SEQ ID NOs: 2-8.

In some aspects, the disclosure provides a host cell comprising the recombinant expression vector, isolated AAV capsid protein, or rAAV as described herein.

### BRIEF DESCRIPTION OF DRAWINGS

FIGS. 1A-1B demonstrate the in vitro packaging efficiency of AAV2 capsid variants identified from human tissue samples. Each histogram shows the relative packaging yield for each AAV2 capsid variant compared to wild-type AAV via small-scale plasmid transfection of HEK-293 cells. Variants that were identified as being able to transduce tissues of the central nervous system (CNS) are marked with asterisks.

FIG. 2 shows a summary workflow for characterizing novel AAV variants by high-throughput tropism screening in mice and non-human primates (NHPs).

FIGS. 3A-3C show quantification of capsid variant transduction profiles following intrahippocampal injections in mice. FIG. 3A shows the detection of vector genomes (DNA) or mRNA transcripts (RNA) in the hippocampus via tabulation of barcoded transgenes with high-throughput sequencing. Relative index detection was normalized to viral vector inputs and scaled to AAV2 values (set to 100). FIG. 3B shows the transcript abundances per vector genome (RNA_{counts}/DNA_{counts}). FIG. 3C shows the detection of vector genomes (DNA) or mRNA transcripts (RNA) in the liver via tabulation of barcoded transgenes. Dashed lines indicate AAV9 detection values.

FIGS. 4A-4B show transduction efficiency in the mouse hippocampus of scAAV-CB6-Egfp vectors packaged using selected AAV2 variants. Adult mice were injected with 3.6E9 viral genomes (vg)/mouse of scAAV-CB6-Egfp vectors packaged with AAV9, AAV2.V46, AAV2.V56, AAV2.V67, AAV2.V81, AAV2.V224, AAV2.v326, or AAV2. V358 via intrahippocampal injections into one hemisphere. FIG. 4A shows native EGFP fluorescence in the hippocampus following transduction with rAAVs containing the different capsid variants. FIG. 4B depicts the different regions of the hippocampus in which EGFP fluorescence was quantified: corpus callosum (CC), cornu ammonis 3 (CA3), fimbria, polymorph layer of the dentage gyrus (DG: PL), and granule cell layer of the dentate gyrus (DG: GCL). FIG. 4C shows quantification of the EGFP fluorescence intensity (in arbitrary units) measured in the different regions of the hippocampus following injections with rAAVs containing AAV9 AAV2.V46, or AAV2.v326 capsid proteins.

FIG. 5 demonstrates that the AAV2 capsid variants described herein have the capacity to penetrate the blood-brain barrier (BBB) and transduce different brain structures. Early postnatal (P1) Mmce were subjected to facial vein injection with rAAVs containing AAV2 capsid variants at 4E11 viral genomes (vg)/mouse. One month post injection, mice were sacrificed, and brains were harvested for cryosectioning. Sections were immunostained with anti-EGFP antibodies to visualize transduced brain cells. The approximate borders of the thalami are outlined in yellow dotted lines.

FIGS. 6A-6N show transduction profiles of AAV2 variants in different regions of the central nervous system (CNS). Mice were injected with barcoded Tough Decoy (bcTuD) vectors packaged with a pooled library of AAV2 variants at 3.7E13 vector genomes (vg)/animal. Vector genomes (FIGS. 6A-6D) and bcTuD transcripts (FIGS. 6E-6H) were detected in tissues and the expression reported as heatmaps in different brain regions (deep cerebellar cortex, hippocampus, motor cortex, deep midbrain, substantia nigra, and VA/VL thalamus). FIGS. 6I-6L show the fold-transcriptional output per vector genome (RNA/DNA) for the same brain regions. Counts were normalized to AAV9 values and scaled to 100 for RNA and DNA values, and 1 for RNA/DNA ratios. Each row represents values detected for each capsid variant. Columns represent tissue type and animal. Variants that were identified as being able to transduce tissues of the central nervous system (CNS) are marked with asterisks. FIGS. 6M-6N show the bcTuD expression in each deep brain structure of non-human primate brains for the seven AAV2 capsid variants described herein. The dotted lines demarcate AAV9 values.

### DETAILED DESCRIPTION OF INVENTION

Adeno-associated virus (AAV) is a small (˜26 nm) replication-defective, non-enveloped virus that generally depends on the presence of a second virus, such as adenovirus or herpes virus, for its growth in cells. AAV is not known to cause disease and induces a very mild immune response. AAV can infect both dividing and non-dividing cells and may incorporate its genome into that of the host cell. These features make AAV a very attractive candidate for creating viral vectors for gene therapy. Prototypical AAV vectors based on serotype 2 provided a proof-of-concept for non-toxic and stable gene transfer in murine and large animal models, but exhibited poor gene transfer efficiency in many major target tissues. The disclosure in some aspects seeks to overcome this shortcoming by providing novel AAVs having distinct tissue targeting capabilities for gene therapy and research applications.

In some aspects of the disclosure, new AAV2 capsid proteins are provided that have distinct tissue targeting capabilities (e.g., tissues of the central nervous system). In some aspects, the disclosure relates to compositions and methods for delivering a transgene (e.g., a transgene encoding one or more gene products) to a target cell (e.g., a cell of the central nervous system, such as a neuron, astrocyte, oligodendrocyte, Muller glial cell, Schwann cell, enteric glial cell, or microglial cell). The disclosure is based, in part, on adeno-associated virus (AAV) capsid protein variants characterized by tropisms for certain types of brain cells. In some embodiments, recombinant AAVs (rAAVs) comprising the capsid protein variants cross the blood-brain barrier (BBB) more efficiently than rAAVs having certain wild-type AAV capsid proteins, for example AAV2 or AAV9 capsid proteins. Methods of delivering an rAAV comprising the AAV2 capsid protein variants are also described by the disclosure.

### Methods for Discovering AAV Capsid Proteins

Much of the biology of AAV is influenced by its capsid. Consequently, methods for discovering novel AAVs have been largely focused on isolating DNA sequences for AAV capsids. A central feature of the adeno-associated virus (AAV) latent life cycle is persistence in the form of integrated and/or episomal genomes in a host cell. Methods used for isolating novel AAVs include PCR-based molecular rescue of latent AAV DNA genomes, infectious virus rescue of latent proviral genomes from tissue DNAs in vitro in the presence of adenovirus helper function, and rescue of circular proviral genomes from tissue DNA by rolling-circle-linear amplification, mediated by an isothermal phage Phi-29 polymerase. All of these isolation methods take advantage of the latency of AAV proviral DNA genomes and focus on rescuing persistent viral genomic DNA.

In some aspects, the disclosure relates to the discovery that novel AAV variants with desirable tissue tropisms can be identified from in vivo tissues of a subject. Without wishing to be bound by any particular theory, the use of in vivo tissue exploits the natural reservoir of genomic diversity observed among viral genomic sequences isolated from tissues of a subject. Thus, in some embodiments, in vivo tissues act as natural incubators for viral (e.g., viral capsid protein) diversity through selective pressure and/or immune evasion.

In some aspects, the disclosure relates to the discovery that PCR products resulting from amplification of AAV DNA (e.g., AAV DNA isolated or extracted from a host cell or in vivo tissue of a subject) can be subjected to high-throughput single-molecule, real-time (SMRT) sequencing to identify novel capsid protein variants. As used herein, “single-molecule, real-time (SMRT) sequencing” refers to a parallelized single-molecule sequencing method, for example, as described by Roberts et al. (2013) Genome Biology 14:405, doi: 10.1186/gb-2013-14-7-405. Without wishing to be bound by any particular theory, the use of SMRT sequencing removes the need to perform viral genome reconstruction and chimera prediction from aligned short-read fragments obtained from other conventional high-throughput genome sequencing methodologies.

Endogenous latent AAV genomes are transcriptionally active in mammalian cells (e.g., cells of nonhuman primate (NHP) tissues such as liver, spleen, and lymph nodes). Without wishing to be bound by any particular theory, it is hypothesized that to maintain AAV persistence in its host, low levels of transcription from AAV genes could be required, and the resulting cap RNA could serve as more suitable and abundant substrates to retrieve functional cap sequences for vector development. Both rep and cap gene transcripts and ability to generate cDNA of cap RNA through reverse transcription (RT) in vitro significantly increases abundance of templates for PCR-based rescue of novel cap sequences from tissues and enhances the sensitivity of novel AAV discovery.

Novel cap sequences may also be identified by transfecting cells with total cellular DNAs isolated from the tissues that harbor proviral AAV genomes at very low abundance. The cells may be further transfected with genes that provide helper virus function (e.g., adenovirus) to trigger and/or boost AAV gene transcription in the transfected cells. In some embodiments, novel cap sequences of the disclosure may be identified by isolating cap mRNA from the transfected cells, creating cDNA from the mRNA (e.g., by RT-PCR), and sequencing the cDNA.

### Isolated Capsid Proteins and Nucleic Acids Encoding the Same

AAVs isolated from mammals, particularly non-human primates (NHPs), are useful for creating gene transfer vectors for clinical development and human gene therapy applications. The disclosure provides, in some aspects, novel AAVs that have been discovered in the brain using the methods disclosed herein. In some embodiments, nucleic acids encoding capsid proteins of these novel AAVs have been discovered in viral genomic DNA isolated from human brain.

The disclosure herein provides adeno-associated virus (AAV) capsid proteins having at least one amino acid mutation at an amino acid position corresponding to position P32, K39, N66, A70, G115, S149, V151, P153, A162, T205, N312, R447, T450, Q457, Q461, S492, E499, P521, S525, F533, G546, E548, K556, R585, R588, or A593 with reference to amino acid position numbering of a wild-type AAV2 capsid protein (e.g., SEQ ID NO: 1), wherein the AAV capsid protein has a tropism for brain cells of the central nervous system. In some aspects, the disclosure provides a method for delivering a transgene to a cell of the central nervous system (e.g., brain cell) in a subject, the method comprising administering to the subject a recombinant adeno-associated virus (rAAV) comprising an isolated nucleic acid comprising a transgene encoding one or more gene products; and an adeno-associated virus (AAV) capsid protein having at least one amino acid mutation at an amino acid position corresponding to position P32, K39, N66, A70, G115, S149, V151, P153, A162, T205, N312, R447, T450, Q457, Q461, S492, E499, P521, S525, F533, G546, E548, K556, R585, R588, or A593 with reference to amino acid position number of a wild-type AAV2 capsid protein (e.g., SEQ ID NO: 1).

In some embodiments, an AAV capsid protein comprises an amino acid mutation at an amino acid position corresponding to position P32. In some embodiments, an amino acid mutation at a position corresponding to position P32 is an amino acid substitution. In some embodiments, an amino acid substitution at position P32 is P32L. In some embodiments, an AAV capsid protein comprises (i) an amino acid mutation at an amino acid position corresponding to position P32, and (ii) at least 90%, 95%, 97%, or 98% sequence homology (or sequence identity) to a wild-type AAV2 capsid protein (e.g., SEQ ID NO: 1).

In some embodiments, an AAV capsid protein comprises an amino acid mutation at an amino acid position corresponding to position K39. In some embodiments, an amino acid mutation at a position corresponding to position K39 is an amino acid substitution. In some embodiments, an amino acid substitution at position K39 is K39Q. In some embodiments, an AAV capsid protein comprises (i) an amino acid mutation at an amino acid position corresponding to position K39, and (ii) at least 90%, 95%, 97%, or 98% sequence homology (or sequence identity) to a wild-type AAV2 capsid protein (e.g., SEQ ID NO: 1).

In some embodiments, an AAV capsid protein comprises an amino acid mutation at an amino acid position corresponding to position N66. In some embodiments, an amino acid mutation at a position corresponding to position N66 is an amino acid substitution. In some embodiments, an amino acid substitution at position N66 is N66S. In some embodiments, an AAV capsid protein comprises (i) an amino acid mutation at an amino acid position corresponding to position N66, and (ii) at least 90%, 95%, 97%, or 98% sequence homology (or sequence identity) to a wild-type AAV2 capsid protein (e.g., SEQ ID NO: 1).

In some embodiments, an AAV capsid protein comprises an amino acid mutation at an amino acid position corresponding to position A70. In some embodiments, an amino acid mutation at a position corresponding to position A70 is an amino acid substitution. In some embodiments, an amino acid substitution at position A70 is A70V. In some embodiments, an AAV capsid protein comprises (i) an amino acid mutation at an amino acid position corresponding to position A70, and (ii) at least 90%, 95%, 97%, or 98% sequence homology (or sequence identity) to a wild-type AAV2 capsid protein (e.g., SEQ ID NO: 1).

In some embodiments, an AAV capsid protein comprises an amino acid mutation at an amino acid position corresponding to position G115. In some embodiments, an amino acid mutation at a position corresponding to position G115 is an amino acid substitution. In some embodiments, an amino acid substitution at position G115 is G115A. In some embodiments, an AAV capsid protein comprises (i) an amino acid mutation at an amino acid position corresponding to position G115, and (ii) at least 90%, 95%, 97%, or 98% sequence homology (or sequence identity) to a wild-type AAV2 capsid protein (e.g., SEQ ID NO: 1).

In some embodiments, an AAV capsid protein comprises an amino acid mutation at an amino acid position corresponding to position S149. In some embodiments, an amino acid mutation at a position corresponding to position S149 is an amino acid substitution. In some embodiments, an amino acid substitution at position S149 is S149F. In some embodiments, an AAV capsid protein comprises (i) an amino acid mutation at an amino acid position corresponding to position S149, and (ii) at least 90%, 95%, 97%, or 98% sequence homology (or sequence identity) to a wild-type AAV2 capsid protein (e.g., SEQ ID NO: 1).

In some embodiments, an AAV capsid protein comprises an amino acid mutation at an amino acid position corresponding to position V151. In some embodiments, an amino acid mutation at a position corresponding to position V151 is an amino acid substitution. In some embodiments, an amino acid substitution at position V151 is V151A. In some embodiments, an AAV capsid protein comprises (i) an amino acid mutation at an amino acid position corresponding to position V151, and (ii) at least 90%, 95%, 97%, or 98% sequence homology (or sequence identity) to a wild-type AAV2 capsid protein (e.g., SEQ ID NO: 1).

In some embodiments, an AAV capsid protein comprises an amino acid mutation at an amino acid position corresponding to position P153. In some embodiments, an amino acid mutation at a position corresponding to position P153 is an amino acid substitution. In some embodiments, an amino acid substitution at position P153 is P153S. In some embodiments, an AAV capsid protein comprises (i) an amino acid mutation at an amino acid position corresponding to position P153, and (ii) at least 90%, 95%, 97%, or 98% sequence homology (or sequence identity) to a wild-type AAV2 capsid protein (e.g., SEQ ID NO: 1).

In some embodiments, an AAV capsid protein comprises an amino acid mutation at an amino acid position corresponding to position A162. In some embodiments, an amino acid mutation at a position corresponding to position A162 is an amino acid substitution. In some embodiments, an amino acid substitution at position A162 is A162S. In some embodiments, an AAV capsid protein comprises (i) an amino acid mutation at an amino acid position corresponding to position A162, and (ii) at least 90%, 95%, 97%, or 98% sequence homology (or sequence identity) to a wild-type AAV2 capsid protein (e.g., SEQ ID NO: 1).

In some embodiments, an AAV capsid protein comprises an amino acid mutation at an amino acid position corresponding to position T205. In some embodiments, an amino acid mutation at a position corresponding to position T205 is an amino acid substitution. In some embodiments, an amino acid substitution at position T205 is T205A. In some embodiments, an amino acid substitution at position T205 is T205S. In some embodiments, an AAV capsid protein comprises (i) an amino acid mutation at an amino acid position corresponding to position T205, and (ii) at least 90%, 95%, 97%, or 98% sequence homology (or sequence identity) to a wild-type AAV2 capsid protein (e.g., SEQ ID NO: 1).

In some embodiments, an AAV capsid protein comprises an amino acid mutation at an amino acid position corresponding to position N312. In some embodiments, an amino acid mutation at a position corresponding to position N312 is an amino acid substitution. In some embodiments, an amino acid substitution at position N312 is N312S. In some embodiments, an AAV capsid protein comprises (i) an amino acid mutation at an amino acid position corresponding to position N312, and (ii) at least 90%, 95%, 97%, or 98% sequence homology (or sequence identity) to a wild-type AAV2 capsid protein (e.g., SEQ ID NO: 1).

In some embodiments, an AAV capsid protein comprises an amino acid mutation at an amino acid position corresponding to position R447. In some embodiments, an amino acid mutation at a position corresponding to position R447 is an amino acid substitution. In some embodiments, an amino acid substitution at position R447 is R447K. In some embodiments, an AAV capsid protein comprises (i) an amino acid mutation at an amino acid position corresponding to position R447, and (ii) at least 90%, 95%, 97%, or 98% sequence homology (or sequence identity) to a wild-type AAV2 capsid protein (e.g., SEQ ID NO: 1).

In some embodiments, an AAV capsid protein comprises an amino acid mutation at an amino acid position corresponding to position T450. In some embodiments, an amino acid mutation at a position corresponding to position T450 is an amino acid substitution. In some embodiments, an amino acid substitution at position T450 is T450A. In some embodiments, an AAV capsid protein comprises (i) an amino acid mutation at an amino acid position corresponding to position T450, and (ii) at least 90%, 95%, 97%, or 98% sequence homology (or sequence identity) to a wild-type AAV2 capsid protein (e.g., SEQ ID NO: 1).

In some embodiments, an AAV capsid protein comprises an amino acid mutation at an amino acid position corresponding to position Q457. In some embodiments, an amino acid mutation at a position corresponding to position Q457 is an amino acid substitution. In some embodiments, an amino acid substitution at position Q457 is Q457M. In some embodiments, an AAV capsid protein comprises (i) an amino acid mutation at an amino acid position corresponding to position Q457, and (ii) at least 90%, 95%, 97%, or 98% sequence homology (or sequence identity) to a wild-type AAV2 capsid protein (e.g., SEQ ID NO: 1).

In some embodiments, an AAV capsid protein comprises an amino acid mutation at an amino acid position corresponding to position Q461. In some embodiments, an amino acid mutation at a position corresponding to position Q461 is an amino acid substitution. In some embodiments, an amino acid substitution at position Q461 is Q461R. In some embodiments, an AAV capsid protein comprises (i) an amino acid mutation at an amino acid position corresponding to position Q461, and (ii) at least 90%, 95%, 97%, or 98% sequence homology (or sequence identity) to a wild-type AAV2 capsid protein (e.g., SEQ ID NO: 1).

In some embodiments, an AAV capsid protein comprises an amino acid mutation at an amino acid position corresponding to position S492. In some embodiments, an amino acid mutation at a position corresponding to position S492 is an amino acid substitution. In some embodiments, an amino acid substitution at position S492 is S492A. In some embodiments, an AAV capsid protein comprises (i) an amino acid mutation at an amino acid position corresponding to position S492, and (ii) at least 90%, 95%, 97%, or 98% sequence homology (or sequence identity) to a wild-type AAV2 capsid protein (e.g., SEQ ID NO: 1).

In some embodiments, an AAV capsid protein comprises an amino acid mutation at an amino acid position corresponding to position P521. In some embodiments, an amino acid mutation at a position corresponding to position P521 is an amino acid substitution. In some embodiments, an amino acid substitution at position P521 is P521T. In some embodiments, an AAV capsid protein comprises (i) an amino acid mutation at an amino acid position corresponding to position P521, and (ii) at least 90%, 95%, 97%, or 98% sequence homology (or sequence identity) to a wild-type AAV2 capsid protein (e.g., SEQ ID NO: 1).

In some embodiments, an AAV capsid protein comprises an amino acid mutation at an amino acid position corresponding to position S525. In some embodiments, an amino acid mutation at a position corresponding to position S525 is an amino acid substitution. In some embodiments, an amino acid substitution at position S525 is S525N. In some embodiments, an AAV capsid protein comprises (i) an amino acid mutation at an amino acid position corresponding to position S525, and (ii) at least 90%, 95%, 97%, or 98% sequence homology (or sequence identity) to a wild-type AAV2 capsid protein (e.g., SEQ ID NO: 1).

In some embodiments, an AAV capsid protein comprises an amino acid mutation at an amino acid position corresponding to position F533. In some embodiments, an amino acid mutation at a position corresponding to position F533 is an amino acid substitution. In some embodiments, an amino acid substitution at position F533 is F533Y. In some embodiments, an AAV capsid protein comprises (i) an amino acid mutation at an amino acid position corresponding to position F533, and (ii) at least 90%, 95%, 97%, or 98% sequence homology (or sequence identity) to a wild-type AAV2 capsid protein (e.g., SEQ ID NO: 1).

In some embodiments, an AAV capsid protein comprises an amino acid mutation at an amino acid position corresponding to position G546. In some embodiments, an amino acid mutation at a position corresponding to position G546 is an amino acid substitution. In some embodiments, an amino acid substitution at position G546 is G546D. In some embodiments, an AAV capsid protein comprises (i) an amino acid mutation at an amino acid position corresponding to position G546, and (ii) at least 90%, 95%, 97%, or 98% sequence homology (or sequence identity) to a wild-type AAV2 capsid protein (e.g., SEQ ID NO: 1).

In some embodiments, an AAV capsid protein comprises an amino acid mutation at an amino acid position corresponding to position E548. In some embodiments, an amino acid mutation at a position corresponding to position E548 is an amino acid substitution. In some embodiments, an amino acid substitution at position E548 is E548G. In some embodiments, an amino acid substitution at position E548 is E548N. In some embodiments, an AAV capsid protein comprises (i) an amino acid mutation at an amino acid position corresponding to position E548, and (ii) at least 90%, 95%, 97%, or 98% sequence homology (or sequence identity) to a wild-type AAV2 capsid protein (e.g., SEQ ID NO: 1).

In some embodiments, an AAV capsid protein comprises an amino acid mutation at an amino acid position corresponding to position K556. In some embodiments, an amino acid mutation at a position corresponding to position K556 is an amino acid substitution. In some embodiments, an amino acid substitution at position K556 is K556R. In some embodiments, an AAV capsid protein comprises (i) an amino acid mutation at an amino acid position corresponding to position K556, and (ii) at least 90%, 95%, 97%, or 98% sequence homology (or sequence identity) to a wild-type AAV2 capsid protein (e.g., SEQ ID NO: 1).

In some embodiments, an AAV capsid protein comprises an amino acid mutation at an amino acid position corresponding to position R585. In some embodiments, an amino acid mutation at a position corresponding to position R585 is an amino acid substitution. In some embodiments, an amino acid substitution at position R585 is R585S. In some embodiments, an AAV capsid protein comprises (i) an amino acid mutation at an amino acid position corresponding to position R585, and (ii) at least 90%, 95%, 97%, or 98% sequence homology (or sequence identity) to a wild-type AAV2 capsid protein (e.g., SEQ ID NO: 1).

In some embodiments, an AAV capsid protein comprises an amino acid mutation at an amino acid position corresponding to position R588. In some embodiments, an amino acid mutation at a position corresponding to position R588 is an amino acid substitution. In some embodiments, an amino acid substitution at position R588 is R588T. In some embodiments, an AAV capsid protein comprises (i) an amino acid mutation at an amino acid position corresponding to position R588, and (ii) at least 90%, 95%, 97%, or 98% sequence homology (or sequence identity) to a wild-type AAV2 capsid protein (e.g., SEQ ID NO: 1).

In some embodiments, an AAV capsid protein comprises an amino acid mutation at an amino acid position corresponding to position A593. In some embodiments, an amino acid mutation at a position corresponding to position A593 is an amino acid substitution. In some embodiments, an amino acid substitution at position A593 is A593T. In some embodiments, an amino acid substation at position A593 is A593S. In some embodiments, an AAV capsid protein comprises (i) an amino acid mutation at an amino acid position corresponding to position A593, and (ii) at least 90%, 95%, 97%, or 98% sequence homology (or sequence identity) to a wild-type AAV2 capsid protein (e.g., SEQ ID NO: 1).

In some embodiments, an AAV capsid protein comprises amino acid mutations at amino acid positions corresponding to positions K39, N66, A70, V151, R447, T450, Q457, S492, E499, F533, G546, E548, R585, R588, and A593 (e.g., K39Q, N66S, A70V, V151A, R447K, T450A, Q457M, S492A, E499D, F533Y, G546D, E548G, R585S, R588T, and A593T) with reference to amino acid position numbering of a wild-type AAV2 capsid protein (e.g., SEQ ID NO: 1). In some embodiments, an AAV capsid protein is AAV2.v46 capsid protein. In some embodiments, an AAV capsid protein comprises or consists of the amino acid sequence set forth in SEQ ID NO: 2.

In some embodiments, an AAV capsid protein comprises amino acid mutations at amino acid positions corresponding to positions K39, S149, V151, R447, T450, Q457, S492, E499, F533, G546, E548, R585, R588, and A593 (e.g., K39Q, S149F, V151A, R447K, T450A, Q457M, S492A, E499D, F533Y, G546D, E548G, R585S, R588T, and A593T) with reference to amino acid position numbering of a wild-type AAV2 capsid protein (e.g., SEQ ID NO: 1). In some embodiments, an AAV capsid protein is AAV2.v56. In some embodiments, an AAV capsid protein comprises or consists of the amino acid sequence set forth in SEQ ID NO: 3.

In some embodiments, an AAV capsid protein comprises amino acid mutations at amino acid positions corresponding to positions K39, A70, V151, R447, T450, Q457, S492, E499, F533, G546, E548, R585, R588, and A593 (e.g., K39Q, A70V, V151A, R447K, T450A, Q457M, S492A, E499D, F533Y, G546D, E548G, R585S, R588T, and A593T) with reference to amino acid position numbering of a wild-type AAV2 capsid protein (e.g., SEQ ID NO: 1). In some embodiments, an AAV capsid protein is AAV2.v67. In some embodiments, an AAV capsid protein comprises or consists of the amino acid sequence set forth in SEQ ID NO: 4.

In some embodiments, an AAV capsid protein comprises amino acid mutations at amino acid positions corresponding to positions P32, K39, V151, T205, R447, T450, Q457, S492, E499, F533, G546, E548, R585, R588, and A593 (e.g., P32L, K39Q, V151A, T205A, R447K, T450A, Q457M, S492A, E499D, F533Y, G546D, E548G, R585S, R588T, and A593T) with reference to amino acid position numbering of a wild-type AAV2 capsid protein (e.g., SEQ ID NO: 1). In some embodiments, an AAV capsid protein is AAV2.v81. In some embodiments, an AAV capsid protein comprises or consists of the amino acid sequence set forth in SEQ ID NO: 5.

In some embodiments, an AAV capsid protein comprises amino acid mutations at amino acid positions corresponding to positions G115, V151, A162, T205, Q461, S492, E499, P521, F533, G546, K556, R585, R588, and A593 (e.g., G115A, V151A, A162S, T205S, Q461R, S492A, E499D, P521T, F533Y, G546D, K556R, R585S, R588T, and A593S) with reference to amino acid position numbering of a wild-type AAV2 capsid protein (e.g., SEQ ID NO: 1). In some embodiments, an AAV capsid protein is AAV2.v224. In some embodiments, an AAV capsid protein comprises or consists of the amino acid sequence set forth in SEQ ID NO: 6.

In some embodiments, an AAV capsid protein comprises amino acid mutations at amino acid positions corresponding to positions N312, R447, T450, Q457, S492, E499, S252, F533, E548, R585, R588, and A593 (e.g., N312S, R447K, T450A, Q457M, S492A, E499D, S525N, F533Y, E548N, R585S, R588T, and A593S) with reference to amino acid position numbering of a wild-type AAV2 capsid protein (e.g., SEQ ID NO: 1). In some embodiments, an AAV capsid protein is AAV2.v326. In some embodiments, an AAV capsid protein comprises or consists of the amino acid sequence set forth in SEQ ID NO: 7.

In some embodiments, an AAV capsid protein comprises amino acid mutations at amino acid positions corresponding to positions P153, N312, R447, T450, Q457, S492, E499, F533, E548, R585, R588, and A593 (e.g., P153S, N312S, R447K, T450A, Q457M, S492A, E499D, F533Y, E548N, R585S, R588T, and A593S) with reference to amino acid position numbering of a wild-type AAV2 capsid protein (e.g., SEQ ID NO: 1). In some embodiments, an AAV capsid protein is AAV2.v358. In some embodiments, an AAV capsid protein comprises or consists of the amino acid sequence set forth in SEQ ID NO: 8.

Mutations contemplated herein, with respect to an amino acid sequence, include, without limitation, substitutions, deletions, and additions. An amino acid “substitution” is a change in a single amino acid relative to a reference amino acid sequence. For example, an amino acid substitution at an amino acid position corresponding to K39 with reference to amino acid position numbering of a wild-type AAV2 capsid protein (e.g., SEQ ID NO: 1) involves a change from Lysine (Lys/K) to another amino acid at that position.

An amino acid substitution may result in a change in charge of the side chain of the amino acid position (e.g., from negatively charged to positively charged). In some embodiments, an amino acid substitution results in a change in polarity or hydrophobicity of the side chain of the amino acid position. In some embodiments, an amino acid substitution is a conservative substitution (e.g., a change from valine to alanine). In some embodiments, an amino acid substitution results in a different amino acid at that position that has an “equivalent” charge, polarity, and or chemical class (defined by the amino acid side chain). Table 1 provides the 20 naturally-occurring amino acids with a description of corresponding charge, polarity, and chemical class. For example, arginine has an equivalent charge to histidine and lysine; an equivalent polarity to asparagine, glutamine, serine, threonine, tyrosine, aspartic acid, glutamic acid, arginine, histidine, and lysine; and an equivalent chemical class/side chain to histidine and lysine.

TABLE 1
Amino Acids

| | | | |
|---|---|---|---|
| Amino acid | Abbreviation | Charge | Polarity |

| | | | | |
|---|---|---|---|---|
| Alanine | Ala | A | uncharged | nonpolar |
| Glycine | Gly | G | uncharged | nonpolar |
| Isoleucine | Ile | I | uncharged | nonpolar |
| Leucine | Leu | L | uncharged | nonpolar |
| Proline | Pro | P | uncharged | nonpolar |
| Valine | Val | V | uncharged | nonpolar |
| Phenylalanine | Phe | F | uncharged | nonpolar |
| Tryptophan | Trp | W | uncharged | nonpolar |
| Cysteine | Cys | C | uncharged | nonpolar |
| Methionine | Met | M | uncharged | nonpolar |
| Asparagine | Asn | N | uncharged | polar |
| Glutamine | Gln | Q | uncharged | polar |
| Serine | Ser | S | uncharged | polar |
| Threonine | Thr | T | uncharged | polar |
| Tyrosine | Tyr | Y | uncharged | polar |
| Aspartic acid | Asp | D | negative | polar |
| Glutamic acid | Glu | E | negative | polar |
| Arginine | Arg | R | positive | polar |
| Histidine | His | H | positive | polar |
| Lysine | Lys | K | positive | polar |

Isolated nucleic acids of the disclosure that encode AAV capsid proteins include any nucleic acid having a sequence as set forth in any one of SEQ ID NOs: 10-16, as well as any nucleic acid having a sequence with substantial homology thereto. In some embodiments, isolated nucleic acids of the disclosure include any nucleic acid having a sequence encoding a polypeptide having a sequence as set forth in any one of SEQ ID NOs: 2-8.

In some embodiments, isolated AAV capsid proteins of the disclosure include any protein having an amino acid sequence as set forth in any one of SEQ ID NOs: 2-8, as well as any protein having substantial homology thereto.

“Homology” refers to the percent identity between two polynucleotides or two polypeptide moieties. The term “substantial homology”, when referring to a nucleic acid or fragment thereof, indicates that, when optimally aligned with appropriate nucleic acid insertions or deletions with another nucleic acid (or its complementary strand), there is nucleotide sequence identity in about 90% to 100% of the aligned sequences. When referring to a polypeptide, or fragment thereof, the term “substantial homology” indicates that, when optimally aligned with appropriate gaps, insertions, or deletions with another polypeptide, there is amino acid identity in about 90% to 100% of the aligned sequences. The term “highly conserved” means at least 80% identity, preferably at least 90% identity, and more preferably over 97% identity. In some cases, “highly conserved” may refer to 100% identity. Identity is readily determined by one of skill in the art by, for example, the use of algorithms and computer programs known by those of skill in the art.

As used herein, alignments between sequences of nucleic acids or polypeptides are performed using any of a variety of publicly or commercially available Multiple Sequence Alignment programs, such as “Clustal W”, accessible through Web Servers on the internet. Alternatively, Vector NTI utilities may also be used. There are also a number of algorithms known in the art that can be used to measure nucleotide sequence identity, including those contained in the programs described above. As another example, polynucleotide sequences can be compared using BLASTN, which provides alignments and percent sequence identity of the regions of the best overlap between the query and search sequences. Similar programs are available for the comparison of amino acid sequences, e.g., the “Clustal X” program, BLASTP. Typically, any of these programs are used at default settings, although one of skill in the art can alter these settings as needed. Alternatively, one of skill in the art can utilize another algorithm or computer program that provides at least the level of identity or alignment as that provided by the referenced algorithms and programs. Alignments may be used to identify corresponding amino acids between two peptides or proteins. A “corresponding amino acid” is an amino acid of a protein or peptide sequence that has been aligned with an amino acid of another protein or peptide sequence. Corresponding amino acids may be identical or non-identical. A corresponding amino acid that is a non-identical amino acid may be referred to as a variant amino acid.

Alternatively, for nucleic acids homology can be determined by hybridization of polynucleotides under conditions that form stable duplexes between homologous regions, followed by digestion with single-stranded-specific nuclease(s), and size determination of the digested fragments. DNA sequences that are substantially homologous can be identified, for example, in a Southern hybridization experiment under conditions as defined for that particular system.

In some aspects, the disclosure relates to an AAV2.v46, AAV2.v56, AAV2.v67, AAV2.v81, AAV2.v224, AAV2.v326, or AAV2.v358 capsid protein (e.g., an isolated nucleic acid encoding an AAV2.v46, AAV2.v56, AAV2.v67, AAV2.v81, AAV2.v224, AAV2.v326, or AAV2.v358 capsid protein, a recombinant adeno-associated virus (rAAV) comprising an AAV2.v46, AAV2.v56, AAV2.v67, AAV2.v81, AAV2.v224, AAV2.v326, or AAV2.v358, etc.), or a capsid protein having substantial homology to any one of AAV2.v46, AAV2.v56, AAV2.v67, AAV2.v81, AAV2.v224, AAV2.v326, or AAV2.v358 capsid proteins. In some embodiments, a capsid protein having substantial homology to an AAV2.v46 capsid protein is at least 50%, 60%, 70%, 80%, 90%, 95%, or 99% identical to the amino acid sequence set forth in SEQ ID NO: 2. In some embodiments, a capsid protein having substantial homology to an AAV2.v56 capsid protein is at least 50%, 60%, 70%, 80%, 90%, 95%, or 99% identical to the amino acid sequence set forth in SEQ ID NO: 3. In some embodiments, a capsid protein having substantial homology to an AAV2.v67 capsid protein is at least 50%, 60%, 70%, 80%, 90%, 95%, or 99% identical to the amino acid sequence set forth in SEQ ID NO: 4. In some embodiments, a capsid protein having substantial homology to an AAV2.v81 capsid protein is at least 50%, 60%, 70%, 80%, 90%, 95%, or 99% identical to the amino acid sequence set forth in SEQ ID NO: 5. In some embodiments, a capsid protein having substantial homology to an AAV2.v224 capsid protein is at least 50%, 60%, 70%, 80%, 90%, 95%, or 99% identical to the amino acid sequence set forth in SEQ ID NO: 6. In some embodiments, a capsid protein having substantial homology to an AAV2.v326 capsid protein is at least 50%, 60%, 70%, 80%, 90%, 95%, or 99% identical to the amino acid sequence set forth in SEQ ID NO: 7. In some embodiments, a capsid protein having substantial homology to an AAV2.v358 capsid protein is at least 50%, 60%, 70%, 80%, 90%, 95%, or 99% identical to the amino acid sequence set forth in SEQ ID NO: 8. In some embodiments, a capsid protein having substantial homology to an AAV2.v46, AAV2.v56, AAV2.v67, AAV2.v81, AAV2.v224, AAV2.v326, or AAV2.v358 capsid protein comprises 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 amino acid substitutions, insertions, or deletions, relative to the amino acid sequences set forth in any one of SEQ ID NOs: 2-8.

The disclosure relates, in some aspects, to the discovery that rAAVs comprising AAV2.v46, AAV2.v56, AAV2.v67, AAV2.v81, AAV2.v224, AAV2.v326, or AAV2.v358 capsid proteins are able to cross the blood-brain barrier of a subject (e.g., mouse, non-human primate). In some embodiments, an rAAV comprising an AAV2.v46, AAV2.v56, AAV2.v67, AAV2.v81, AAV2.v224, AAV2.v326, or AAV2.v358 capsid protein crosses the blood-brain barrier of a subject (e.g., mouse, non-human primate, human, etc.) more efficiently than an rAAV having a wild-type AAV2 or AAV9 capsid. In some embodiments, an rAAV comprising an AAV2.v46, AAV2.v56, AAV2.v67, AAV2.v81, AAV2.v224, AAV2.v326, or AAV2.v358 capsid protein crosses the blood-brain barrier of a subject at least 5%, 10%, 15%, 20%, 25%, 30%, 40%, 50%, 100%, 200%, 500%, 1000% or more, more efficiently than rAAVs having a wild-type AAV2 or AAV9 capsid protein.

As used herein, the term “blood-brain barrier” (BBB) is used in reference to the unique properties of the microvasculature of the central nervous system (CNS). CNS vessels are continuous, non-fenestrated vessels that contain a series of additional properties which allow them to tightly regulate the movement of molecules, ions, and cells between the blood and the CNS. This selectivity arises from the epithelial-like tight junctions within the brain capillary endothelium. The BBB is anatomically and functionally distinct from the blood-cerebrospinal fluid barrier at the choroid plexus. This heavily restricting barrier capacity allows BBB endothelial cells to tightly regulate CNS homeostasis, which is critical to allow for proper neuronal function, as well as protect the CNS from toxins, pathogens, inflammation, injury, and disease. BBB dysfunction can lead to ion dysregulation, altered signaling homeostasis, as well as the entry of immune cells and molecules into the CNS, processes that lead to neuronal dysfunction and degeneration. The restrictive nature of the BBB, while imperative for proper physiological functions, provides an obstacle for delivering therapies to the CNS and, thus, must be addressed when developing therapies targeting the CNS.

There are several ways to test whether a compound crosses the BBB. For example, microdialysis, autoradiography, immunohistochemistry, and even whole-brain homogenization can be used in animal models. Because these methods are not feasible in human subjects, clinical trials use other techniques such as positron emission tomography (PET), single-photon emission computerized tomography, and cerebrospinal fluid (CSF) sampling to indirectly measure BBB penetration.

The disclosure relates, in some aspects, to the discovery that rAA Vs comprising AAV2.v46, AAV2.v56, AAV2.v67, AAV2.v81, AAV2.v224, AAV2.v326, or AAV2.v358 capsid proteins efficiently transduce deep brain structures of a subject (e.g., central nervous system [CNS] structures, such as, but not limited to, deep cerebellar cortex, hippocampus, motor cortex, deep midbrain, substantia nigra, or VA/VL thalamus structures). In some embodiments, an rAAV comprising an AAV2.v46, AAV2.v56, AAV2.v67, AAV2.v81, AAV2.v224, AAV2.v326, or AAV2.v358 capsid protein transduces a deep brain structure of a subject more efficiently than an rAAV having a wild-type AAV2 or AAV9 capsid protein. In some embodiments, an rAAV comprising an AAV2.v46, AAV2.v56, AAV2.v67, AAV2.v81, AAV2.v224, AAV2.v326, or AAV2.v358 capsid protein transduces a deep brain structure of a subject at least 5%, 10%, 15%, 20%, 25%, 30%, 40%, 50%, 100%, 200%, 500%, 1000% or more, more efficiently than an rAAV having a wild-type AAV2 or AAV9 capsid protein. In some embodiments, a deep brain structure comprises cells selected from the group comprising neurons, astrocytes, oligodendrocytes, Muller glial cells, Schwann cells, enteric glial cells, or microglial cells.

Aspects of the disclosure relate to certain AAV capsid proteins that are serologically distinct from other AAV capsid protein (e.g., AAV1, AAV2, AAV3B, AAV8, AAV9, AAVrh.8, AAVrh.10, etc.). Without wishing to be bound by any particular theory, rAAVs comprising AAV2.v46, AAV2.v56, AAV2.v67, AAV2.v81, AAV2.v224, AAV2.v326, or AAV2.v358 capsid proteins are not subject to the neutralizing antibody response in a subject that is seropositive for antibodies against certain other AAV capsids. Accordingly, in some embodiments, rAAVs comprising capsid proteins as described herein may be useful as second-line therapy for delivery of transgenes to subjects that have previously been administered AAV therapies, or that are seropositive for certain AAV capsid neutralizing antibodies.

In some aspects, the disclosure relates to isolated nucleic acids encoding certain AAV capsid protein variants (e.g., AAV2.v46, AAV2.v56, AAV2.v67, AAV2.v81, AAV2.v224, AAV2.v326, or AAV2.v358 capsid protein). A “nucleic acid” sequence refers to a DNA or RNA sequence. In some embodiments, the term nucleic acid captures sequences that include any of the known base analogues of DNA and RNA such as, but not limited to 4-acetylcytosine, 8-hydroxy-N6-methyladenosine, aziridinylcytosine, pseudoisocytosine, 5-(carboxyhydroxyl-methyl) uracil, 5-fluorouracil, 5-bromouracil, 5-carboxymethylaminomethyl-2-thiouracil, 5-carboxymethyl-aminomethyluracil, dihydrouracil, inosine, N6-isopentenyladenine, 1-methyladenine, 1-methylpseudo-uracil, 1-methylguanine, 1-methylinosine, 2,2-dimethyl-guanine, 2-methyladenine, 2-methylguanine, 3-methyl-cytosine, 5-methylcytosine, N6-methyladenine, 7-methylguanine, 5-methylaminomethyluracil, 5-methoxy-amino-methyl-2-thiouracil, beta-D-mannosylqueosine, 5′-methoxycarbonylmethyluracil, 5-methoxyuracil, 2-methylthio-N6-isopentenyladenine, uracil-5-oxyacetic acid methylester, uracil-5-oxyacetic acid, oxybutoxosine, pseudouracil, queosine, 2-thiocytosine, 5-methyl-2-thiouracil, 2-thiouracil, 4-thiouracil, 5-methyluracil, -uracil-5-oxyacetic acid methylester, uracil-5-oxyacetic acid, pseudouracil, queosine, 2-thiocytosine, and 2,6-diaminopurine.

In some embodiments, proteins and nucleic acids of the disclosure are isolated. As used herein, the term “isolated” means artificially obtained or produced. As used herein with respect to nucleic acids, the term “isolated” generally means: (i) amplified in vitro by, for example, polymerase chain reaction (PCR); (ii) recombinantly produced by cloning; (iii) purified, as by cleavage and gel separation; or (iv) synthesized by, for example, chemical synthesis. An isolated nucleic acid is one that is readily manipulable by recombinant DNA techniques well known in the art. Thus, a nucleotide sequence contained in a vector in which 5′ and 3′ restriction sites are known or for which polymerase chain reaction (PCR) primer sequences have been disclosed is considered isolated but a nucleic acid sequence existing in its native state in its natural host is not. An isolated nucleic acid may be substantially purified, but need not be. For example, a nucleic acid that is isolated within a cloning or expression vector is not pure in that it may comprise only a tiny percentage of the material in the cell in which it resides. Such a nucleic acid is isolated, however, as the term is used herein because it is readily manipulable by standard techniques known to those of ordinary skill in the art. As used herein with respect to proteins or peptides, the term “isolated” generally refers to a protein or peptide that has been artificially obtained or produced (e.g., by chemical synthesis, by recombinant DNA technology, etc.).

It should be appreciated that conservative amino acid substitutions may be made to provide functionally equivalent variants, or homologs of the capsid proteins. In some aspects the disclosure embraces sequence alterations that result in conservative amino acid substitutions. As used herein, a conservative amino acid substitution refers to an amino acid substitution that does not alter the relative charge or size characteristics of the protein in which the amino acid substitution is made. Variants can be prepared according to methods for altering polypeptide sequence known to one of ordinary skill in the art such as are found in references that compile such methods, e.g., Molecular Cloning: A Laboratory Manual, J. Sambrook, et al., eds., Second Edition, Cold Spring Harbor Laboratory Press, Cold Spring Harbor, New York, 1989, or Current Protocols in Molecular Biology, F. M. Ausubel, et al., eds., John Wiley & Sons, Inc., New York. Conservative substitutions of amino acids include substitutions made among amino acids within the following groups: (a) M, I, L, V; (b) F, Y, W; (c) K, R, H; (d) A, G; (e) S, T; (f) Q, N; and (g) E, D. Therefore, one can make conservative amino acid substitutions to the amino acid sequence of the proteins and polypeptides disclosed herein.

An example of an isolated nucleic acid that encodes a polypeptide comprising an AAV capsid protein is a nucleic acid comprising a sequence selected from the group consisting of SEQ ID NOs: 10-16. A fragment of an isolated nucleic acid encoding an AAV capsid sequence may be useful for constructing a nucleic acid encoding a desired capsid sequence. Fragments may be of any appropriate length. In some embodiments a fragment (portion) of an isolated nucleic acid encoding an AAV capsid sequence may be useful for constructing a nucleic acid encoding a desired capsid sequence. Fragments may be of any appropriate length (e.g., at least 6, at least 9, at least 18, at least 36, at least 72, at least 144, at least 288, at least 576, at least 1152 or more nucleotides in length). For example, a fragment of nucleic acid sequence encoding a polypeptide of a first AAV capsid protein may be used to construct, or may be incorporated within, a nucleic acid sequence encoding a second AAV capsid sequence to alter the properties of the AAV capsid. In some embodiments, AAV capsid proteins that comprise capsid sequence fragments from multiple AAV serotypes are referred to as chimeric AAV capsids. The fragment may be a fragment that does not encode a peptide that is identical to a sequence of any one of SEQ ID NOs: 2-8. For example, a fragment of a nucleic acid sequence encoding a variant amino acid (compared with a known wild-type AAV serotype) may be used to construct, or may be incorporated within, a nucleic acid sequence encoding an AAV capsid sequence to alter the properties of the AAV capsid.

Recombinant AAVs (rAAVs)

In some aspects, the disclosure provides isolated AAVs. As used herein with respect to AAVs, the term “isolated” refers to an AAV that has been artificially obtained or produced. Isolated AAVs may be produced using recombinant methods. Such AAVs are referred to herein as “recombinant AAVs”. Recombinant AAVs (rAAVs) preferably have tissue-specific targeting capabilities, such that a transgene of the rAAV will be delivered specifically to one or more predetermined tissue(s). The AAV capsid is an important element in determining these tissue-specific targeting abilities. Thus, an rAAV having a capsid appropriate for the tissue being targeted can be selected. In some embodiments, the rAAV comprises a capsid protein having an amino acid sequence as set forth in any one of SEQ ID NOs: 2-8, or a protein having substantial homology thereto.

Methods for obtaining recombinant AAVs having a desired capsid protein are well known in the art. (See, for example, US 2003/0138772), the contents of which are incorporated herein by reference in their entirety). Typically the methods involve culturing a host cell which contains a nucleic acid sequence encoding an AAV capsid protein (e.g., a nucleic acid encoding a polypeptide having a sequence as set forth in any one of SEQ ID NOs 1-409, 435-868, or 1726-1988) or fragment thereof; a functional rep gene; a recombinant AAV vector composed of, AAV inverted terminal repeats (ITRs) and a transgene; and sufficient helper functions to permit packaging of the recombinant AAV vector into the AAV capsid proteins. In some embodiments, capsid proteins are structural proteins encoded by a cap gene of an AAV. In some embodiments, AAVs comprise three capsid proteins, virion proteins 1 to 3 (named VP1, VP2 and VP3), all of which may be expressed from a single cap gene. Accordingly, in some embodiments, the VP1, VP2 and VP3 proteins share a common core sequence. In some embodiments, the molecular weights of VP1, VP2 and VP3 are respectively about 87 kDa, about 72 kDa and about 62 kDa. In some embodiments, upon translation, capsid proteins form a spherical 60-mer protein shell around the viral genome. In some embodiments, the protein shell is primarily comprised of a VP3 capsid protein. In some embodiments, the functions of the capsid proteins are to protect the viral genome, deliver the genome and interact with the host. In some aspects, capsid proteins deliver the viral genome to a host in a tissue specific manner. In some embodiments, VP1 and/or VP2 capsid proteins may contribute to the tissue tropism of the packaged AAV. In some embodiments, the tissue tropism of the packaged AAV is determined by the VP3 capsid protein. In some embodiments, the tissue tropism of an AAV is enhanced or changed by mutations occurring in the capsid proteins.

In some embodiments, AAV variants described herein may be useful for delivering gene therapy to tissue or cells of the central nervous system (e.g., brain tissue or cells). Accordingly, in some embodiments, AAV variants described herein may be useful for the treatment of disorders affecting the central nervous system (e.g., brain). A disorder of the CNS (e.g., brain disorder) may affect the deep cerebellar cortex, hippocampus, motor cortex, deep midbrain, substantia nigra, or VA/VL thalamus. A disorder of the CNS (e.g., brain disorder) may be of a genetic origin, either inherited or acquired through a somatic mutation. Non-limiting examples of disorders and diseases affecting the CNS (e.g., brain) include, but are not limited to: Huntington's disease, Neurofibromatosis Type 1, Neurofibromatosis Type 2, Polycystic Kidney Disease, Tay-Sachs Disease, Tuberous Sclerosis, von Hippel Lindau Syndrome, Leukodystrophy, Phenylketonuria, and Wilson disease.

“Deep brain” as used herein refers to nucleus structures of the brain comprising clusters of neurons that are situated deep within the cerebral hemispheres of the brain. Structures of the deep brain include, but are not limited to, the corpus callosum, cornu ammonis, fimbria, polymorph layer of the dentate gyrus, and granule cell layer of the dentate gyrus of the hippocampus, the deep cerebellar cortex, motor cortex, deep midbrain, substantia nigra, ventral anterior nucleus, and ventral lateral nucleus.

The components to be cultured in the host cell to package a rAAV vector in an AAV capsid may be provided to the host cell in trans. Alternatively, any one or more of the required components (e.g., recombinant AAV vector, rep sequences, cap sequences, and/or helper functions) may be provided by a stable host cell which has been engineered to contain one or more of the required components using methods known to those of skill in the art. Most suitably, such a stable host cell will contain the required component(s) under the control of an inducible promoter. However, the required component(s) may be under the control of a constitutive promoter. Examples of suitable inducible and constitutive promoters are provided herein, in the discussion of regulatory elements suitable for use with the transgene. In still another alternative, a selected stable host cell may contain selected component(s) under the control of a constitutive promoter and other selected component(s) under the control of one or more inducible promoters. For example, a stable host cell may be generated which is derived from 293 cells (which contain E1 helper functions under the control of a constitutive promoter), but which contain the rep and/or cap proteins under the control of inducible promoters. Still other stable host cells may be generated by one of skill in the art.

The recombinant AAV vector, rep sequences, cap sequences, and helper functions required for producing the rAAV of the disclosure may be delivered to the packaging host cell using any appropriate genetic element (vector). In some embodiments, a single nucleic acid encoding all three capsid proteins (e.g., VP1, VP2 and VP3) is delivered into the packaging host cell in a single vector. In some embodiments, nucleic acids encoding the capsid proteins are delivered into the packaging host cell by two vectors; a first vector comprising a first nucleic acid encoding two capsid proteins (e.g., VP1 and VP2) and a second vector comprising a second nucleic acid encoding a single capsid protein (e.g., VP3). In some embodiments, three vectors, each comprising a nucleic acid encoding a different capsid protein, are delivered to the packaging host cell. The selected genetic element may be delivered by any suitable method, including those described herein. The methods used to construct any embodiment of this disclosure are known to those with skill in nucleic acid manipulation and include genetic engineering, recombinant engineering, and synthetic techniques. See, e.g., Sambrook et al, Molecular Cloning: A Laboratory Manual, Cold Spring Harbor Press, Cold Spring Harbor, N.Y. Similarly, methods of generating rAAV virions are well known and the selection of a suitable method is not a limitation on the present disclosure. See, e.g., K. Fisher et al, J. Virol., 70:520-532 (1993) and U.S. Pat. No. 5,478,745.

In some embodiments, rAAVs may be produced using the triple transfection method (described in detail in U.S. Pat. No. 6,001,650). Typically, the recombinant AAVs are produced by transfecting a host cell with a recombinant AAV vector (comprising a transgene) to be packaged into AAV particles, an AAV helper function vector, and an accessory function vector. An AAV helper function vector encodes the “AAV helper function” sequences (e.g., rep and cap), which function in trans for productive AAV replication and encapsidation. Preferably, the AAV helper function vector supports efficient AAV vector production without generating any detectable wild-type AAV virions (e.g., AAV virions containing functional rep and cap genes). Non-limiting examples of vectors suitable for use with the present disclosure include pHLP19, described in U.S. Pat. No. 6,001,650 and pRep6cap6 vector, described in U.S. Pat. No. 6,156,303, the entirety of both incorporated by reference herein. The accessory function vector encodes nucleotide sequences for non-AAV derived viral and/or cellular functions upon which AAV is dependent for replication (e.g., “accessory functions”). The accessory functions include those functions required for AAV replication, including, without limitation, those moieties involved in activation of AAV gene transcription, stage specific AAV mRNA splicing, AAV DNA replication, synthesis of cap expression products, and AAV capsid assembly. Viral-based accessory functions can be derived from any of the known helper viruses such as adenovirus, herpesvirus (other than herpes simplex virus type-1), and vaccinia virus.

In some aspects, the disclosure provides transfected host cells. The term “transfection” is used to refer to the uptake of foreign DNA by a cell, and a cell has been “transfected” when exogenous DNA has been introduced inside the cell (e.g., across the cell membrane). A number of transfection techniques are generally known in the art. See, e.g., Graham et al. (1973) Virology, 52:456, Sambrook et al. (1989) Molecular Cloning, a laboratory manual, Cold Spring Harbor Laboratories, New York, Davis et al. (1986) Basic Methods in Molecular Biology, Elsevier, and Chu et al. (1981) Gene 13:197. Such techniques can be used to introduce one or more exogenous nucleic acids, such as a nucleotide integration vector and other nucleic acid molecules, into suitable host cells.

A “host cell” refers to any cell that harbors, or is capable of harboring, a substance of interest. Often a host cell is a mammalian cell. A host cell may be used as a recipient of an AAV helper construct, an AAV minigene plasmid, an accessory function vector, or other transfer DNA associated with the production of rAAVs. The term includes the progeny of the original cell that has been transfected. Thus, a “host cell” as used herein may refer to a cell that has been transfected with an exogenous DNA sequence. It is understood that the progeny of a single parental cell may not necessarily be completely identical in morphology or in genomic or total DNA complement as the original parent, due to natural, accidental, or deliberate mutation.

As used herein, the term “cell line” refers to a population of cells capable of continuous or prolonged growth and division in vitro. Often, cell lines are clonal populations derived from a single progenitor cell. It is further known in the art that spontaneous or induced changes can occur in karyotype during storage or transfer of such clonal populations. Therefore, cells derived from the cell line referred to may not be precisely identical to the ancestral cells or cultures, and the cell line referred to includes such variants.

As used herein, the terms “recombinant cell” refers to a cell into which an exogenous DNA segment, such as DNA segment that leads to the transcription of a biologically-active polypeptide or production of a biologically active nucleic acid such as an RNA, has been introduced.

Cells may also be transfected with a vector (e.g., helper vector) that provides helper functions to the AAV. The vector providing helper functions may provide adenovirus functions, including, e.g., E1a, E1b, E2a, and E4ORF6. The sequences of adenovirus gene providing these functions may be obtained from any known adenovirus serotype, such as serotypes 2, 3, 4, 7, 8, 9, 12 and 40, and further including any of the presently identified human types known in the art. Thus, in some embodiments, the methods involve transfecting the cell with a vector expressing one or more genes necessary for AAV replication, AAV gene transcription, and/or AAV packaging.

As used herein, the term “vector” includes any genetic element, such as a plasmid, phage, transposon, cosmid, chromosome, artificial chromosome, virus, virion, etc., that is capable of replication when associated with the proper control elements and which can transfer gene sequences between cells. Thus, the term includes cloning and expression vehicles, as well as viral vectors. In some embodiments, useful vectors are contemplated to be those vectors in which the nucleic acid segment (e.g., nucleic acid sequence) to be transcribed is positioned under the transcriptional control of a promoter. A “promoter” refers to a DNA sequence recognized by the synthetic machinery of the cell, or introduced synthetic machinery, that is required to initiate the specific transcription of a gene. The phrases “operatively positioned,” “under control” or “under transcriptional control” means that the promoter is in the correct location and orientation in relation to the nucleic acid to control RNA polymerase initiation and expression of the gene. The term “expression vector or construct” means any type of genetic construct containing a nucleic acid in which part or all of the nucleic acid encoding sequence is capable of being transcribed. In some embodiments, expression includes transcription of the nucleic acid, for example, to generate a biologically-active polypeptide product or inhibitory RNA (e.g., shRNA, miRNA, miRNA inhibitor) from a transcribed gene.

In some cases, an isolated capsid gene can be used to construct and package recombinant AAVs, using methods well known in the art, to determine functional characteristics associated with the capsid protein encoded by the gene. For example, isolated capsid genes can be used to construct and package a recombinant AAV (rAAV) comprising a reporter gene (e.g., B-Galactosidase, GFP, Luciferase, etc.). The rAAV can then be delivered to an animal (e.g., mouse) and the tissue targeting properties of the novel isolated capsid gene can be determined by examining the expression of the reporter gene in various tissues (e.g., heart, liver, kidneys) of the animal. Other methods for characterizing the novel isolated capsid genes are disclosed herein and still others are well known in the art.

The foregoing methods for packaging recombinant vectors in desired AAV capsids to produce the rAAVs of the disclosure are not meant to be limiting and other suitable methods will be apparent to the skilled artisan.

rAAV Vectors

“Recombinant AAV (rAAV) vectors” of the disclosure are typically composed of, at a minimum, a transgene (e.g., a transgene encoding one or more gene products) and its regulatory sequences, and 5′ and 3′ AAV inverted terminal repeats (ITRs). It is this recombinant AAV vector which is packaged into a capsid protein and delivered to a selected target cell. In some embodiments, the transgene is a nucleic acid sequence, heterologous to the vector sequences, that encodes a polypeptide, protein, functional RNA molecule (e.g., miRNA, miRNA inhibitor) or other gene product, of interest. The nucleic acid coding sequence is operatively linked to regulatory components in a manner that permits transgene transcription, translation, and/or expression in a cell of a target tissue.

The AAV sequences of the vector typically comprise the cis-acting 5′ and 3′ inverted terminal repeat sequences (See, e.g., B. J. Carter, in “Handbook of Parvoviruses”, ed., P. Tijsser, CRC Press, pp. 155 168 (1990)). The ITR sequences are about 145 bp in length. Preferably, substantially the entire sequences encoding the ITRs are used in the molecule, although some degree of minor modification of these sequences is permissible. The ability to modify these ITR sequences is within the skill of the art. (See, e.g., texts such as Sambrook et al, “Molecular Cloning. A Laboratory Manual”, 2d ed., Cold Spring Harbor Laboratory, New York (1989); and K. Fisher et al., J Virol., 70:520 532 (1996)). An example of such a molecule employed in the present disclosure is a “cis-acting” plasmid containing the transgene, in which the selected transgene sequence and associated regulatory elements are flanked by the 5′ and 3′ AAV ITR sequences. The AAV ITR sequences may be obtained from any known AAV, including presently identified mammalian AAV types.

In some embodiments, the disclosure provides a self-complementary AAV vector. As used herein, the term “self-complementary AAV vector” (scAAV) refers to a vector containing a double-stranded vector genome generated by the absence of a terminal resolution site (TR) from one of the ITRs of the AAV. The absence of a TR prevents the initiation of replication at the vector terminus where the TR is not present. In general, scAAV vectors generate single-stranded, inverted repeat genomes, with a wild-type (wt) AAV TR at each end and a mutated TR (mTR) in the middle.

In some embodiments, the rAAVs of the present disclosure are pseudotyped rAAVs. Pseudotyping is the process of producing viruses or viral vectors in combination with foreign viral envelope proteins. The result is a pseudotyped virus particle. With this method, the foreign viral envelope proteins can be used to alter host tropism or an increased/decreased stability of the virus particles. In some aspects, a pseudotyped rAAV comprises nucleic acids from two or more different AAVs, wherein the nucleic acid from one AAV encodes a capsid protein and the nucleic acid of at least one other AAV encodes other viral proteins and/or the viral genome. In some embodiments, a pseudotyped rAAV refers to an AAV comprising an inverted terminal repeat (ITR) of one AAV serotype and a capsid protein of a different AAV serotype. For example, a pseudotyped AAV vector containing the ITRs of serotype X encapsidated with the proteins of Y will be designated as AAVX/Y (e.g., AAV2/1 has the ITRs of AAV2 and the capsid of AAV1). In some embodiments, pseudotyped rAAVs may be useful for combining the tissue-specific targeting capabilities of a capsid protein from one AAV serotype with the viral DNA from another AAV serotype, thereby allowing targeted delivery of a transgene to a target tissue.

In addition to the major elements identified above for the recombinant AAV vector, the vector also includes conventional control elements necessary which are operably linked to the transgene in a manner which permits its transcription, translation and/or expression in a cell transfected with the plasmid vector or infected with the virus produced by the disclosure. As used herein, “operably linked” sequences include both expression control sequences that are contiguous with the gene of interest and expression control sequences that act in trans or at a distance to control the gene of interest.

Expression control sequences include appropriate transcription initiation, termination, promoter and enhancer sequences; efficient RNA processing signals such as splicing and polyadenylation (polyA) signals; sequences that stabilize cytoplasmic mRNA; sequences that enhance translation efficiency (e.g., Kozak consensus sequence); sequences that enhance protein stability; and when desired, sequences that enhance secretion of the encoded product. A great number of expression control sequences, including promoters that are native, constitutive, inducible and/or tissue-specific, are known in the art and may be utilized.

As used herein, a nucleic acid sequence (e.g., coding sequence) and regulatory sequences are said to be “operably” linked when they are covalently linked in such a way as to place the expression or transcription of the nucleic acid sequence under the influence or control of the regulatory sequences. If it is desired that the nucleic acid sequences be translated into a functional protein, two DNA sequences are said to be operably linked if induction of a promoter in the 5′ regulatory sequences results in the transcription of the coding sequence and if the nature of the linkage between the two DNA sequences does not (1) result in the introduction of a frame-shift mutation, (2) interfere with the ability of the promoter region to direct the transcription of the coding sequences, or (3) interfere with the ability of the corresponding RNA transcript to be translated into a protein. Thus, a promoter region would be operably linked to a nucleic acid sequence if the promoter region were capable of effecting transcription of that DNA sequence such that the resulting transcript might be translated into the desired protein or polypeptide. Similarly two or more coding regions are operably linked when they are linked in such a way that their transcription from a common promoter results in the expression of two or more proteins having been translated in frame. In some embodiments, operably linked coding sequences yield a fusion protein. In some embodiments, operably linked coding sequences yield a functional RNA (e.g., shRNA, miRNA, miRNA inhibitor).

For nucleic acids encoding proteins, a polyadenylation sequence generally is inserted following the transgene sequences and before the 3′ AAV ITR sequence. A rAAV construct useful in the present disclosure may also contain an intron, desirably located between the promoter/enhancer sequence and the transgene. One possible intron sequence is derived from SV-40, and is referred to as the SV-40 T intron sequence. Another vector element that may be used is an internal ribosome entry site (IRES). An IRES sequence is used to produce more than one polypeptide from a single gene transcript. An IRES sequence would be used to produce a protein that contains more than one polypeptide chains. Selection of these and other common vector elements are conventional and many such sequences are available [see, e.g., Sambrook et al, and references cited therein at, for example, pages 3.18 3.26 and 16.17 16.27 and Ausubel et al., Current Protocols in Molecular Biology, John Wiley & Sons, New York, 1989]. In some embodiments, a Foot and Mouth Disease Virus 2A sequence is included in polyprotein; this is a small peptide (approximately 18 amino acids in length) that has been shown to mediate the cleavage of polyproteins (Ryan, M D et al., EMBO, 1994; 4:928-933; Mattion, N M et al., J Virology, November 1996; p. 8124-8127; Furler, S et al., Gene Therapy, 2001; 8:864-873; and Halpin, C et al., The Plant Journal, 1999; 4:453-459). The cleavage activity of the 2A sequence has previously been demonstrated in artificial systems including plasmids and gene therapy vectors (AAV and retroviruses) (Ryan, M D et al., EMBO, 1994; 4:928-933; Mattion, N M et al., J Virology, November 1996; p. 8124-8127; Furler, S et al., Gene Therapy, 2001; 8:864-873; and Halpin, C et al., The Plant Journal, 1999; 4:453-459; de Felipe, P et al., Gene Therapy, 1999; 6:198-208; de Felipe, P et al., Human Gene Therapy, 2000; 11:1921-1931; and Klump, H et al., Gene Therapy, 2001; 8:811-817).

The precise nature of the regulatory sequences needed for gene expression in host cells may vary between species, tissues or cell types, but shall in general include, as necessary, 5′ non-transcribed and 5′ non-translated sequences involved with the initiation of transcription and translation respectively, such as a TATA box, capping sequence, CAAT sequence, enhancer elements, and the like. Especially, such 5′ non-transcribed regulatory sequences will include a promoter region that includes a promoter sequence for transcriptional control of the operably joined gene. Regulatory sequences may also include enhancer sequences or upstream activator sequences as desired. The vectors of the disclosure may optionally include 5′ leader or signal sequences. The choice and design of an appropriate vector is within the ability and discretion of one of ordinary skill in the art.

Examples of constitutive promoters include, without limitation, the retroviral Rous sarcoma virus (RSV) LTR promoter (optionally with the RSV enhancer), the cytomegalovirus (CMV) promoter (optionally with the CMV enhancer) [see, e.g., Boshart et al, Cell, 41:521-530 (1985)], the SV40 promoter, the dihydrofolate reductase promoter, the β-actin promoter, the phosphoglycerol kinase (PGK) promoter, and the EF1α promoter [Invitrogen].

Inducible promoters allow regulation of gene expression and can be regulated by exogenously supplied compounds, environmental factors such as temperature, or the presence of a specific physiological state, e.g., acute phase, a particular differentiation state of the cell, or in replicating cells only. Inducible promoters and inducible systems are available from a variety of commercial sources, including, without limitation, Invitrogen, Clontech and Ariad. Many other systems have been described and can be readily selected by one of skill in the art. Examples of inducible promoters regulated by exogenously supplied promoters include the zinc-inducible sheep metallothionine (MT) promoter, the dexamethasone (Dex)-inducible mouse mammary tumor virus (MMTV) promoter, the T7 polymerase promoter system (WO 98/10088); the ecdysone insect promoter (No et al, Proc. Natl. Acad. Sci. USA, 93:3346-3351 (1996)), the tetracycline-repressible system (Gossen et al, Proc. Natl. Acad. Sci. USA, 89:5547-5551 (1992)), the tetracycline-inducible system (Gossen et al, Science, 268:1766-1769 (1995), see also Harvey et al, Curr. Opin. Chem. Biol., 2:512-518 (1998)), the RU486-inducible system (Wang et al, Nat. Biotech., 15:239-243 (1997) and Wang et al, Gene Ther., 4:432-441 (1997)) and the rapamycin-inducible system (Magari et al, J. Clin. Invest., 100:2865-2872 (1997)). Still other types of inducible promoters that may be useful in this context are those that are regulated by a specific physiological state, e.g., temperature, acute phase, a particular differentiation state of the cell, or in replicating cells only.

In another embodiment, the native promoter for the transgene will be used. The native promoter may be preferred when it is desired that expression of the transgene should mimic the native expression. The native promoter may be used when expression of the transgene must be regulated temporally or developmentally, or in a tissue-specific manner, or in response to specific transcriptional stimuli. In a further embodiment, other native expression control elements, such as enhancer elements, polyadenylation sites or Kozak consensus sequences may also be used to mimic the native expression.

In some embodiments, a nucleic acid described herein utilizes a tissue-specific promoter (e.g., to promote expression of a transgene in a tissue-specific manner). In some embodiments, a tissue-specific promoter is a central nervous system (e.g., brain)-specific promoter. Examples of CNS-specific promoters include glial fibrillary acidic protein (GFAP) promoter, gfaABC1D promoter, gfa28/gfaABD promoter, ALDH1L1 promoter, gfa2 promoter, gfa2(B₃) promoter, Mbp promoter, MAG promoter, Cbh promoter, F4/80 promoter, CD68 promoter, CD11B promoter, RLBP1 promoter, ProB2 promoter, Mpz promoter, or Cnp promoter.

In some embodiments, the regulatory sequences impart tissue-specific gene expression capabilities. In some cases, the tissue-specific regulatory sequences bind tissue-specific transcription factors that induce transcription in a tissue specific manner. Such tissue-specific regulatory sequences (e.g., promoters, enhancers, etc.) are well known in the art. Exemplary tissue-specific regulatory sequences include, but are not limited to the following tissue specific promoters: a liver-specific thyroxin binding globulin (TBG) promoter, an insulin promoter, a glucagon promoter, a somatostatin promoter, a pancreatic polypeptide (PPY) promoter, a synapsin-1 (Syn) promoter, a creatine kinase (MCK) promoter, a mammalian desmin (DES) promoter, a α-myosin heavy chain (a-MHC) promoter, a gastrointestinal-specific mucin-2 promoter, an eye-specific retinoschisin promoter, an eye-specific K12 promoter, a respiratory tissue-specific CC10 promoter, a respiratory tissue-specific surfactant protein C (SP-C) promoter, a breast tissue-specific PRC1 promoter, a breast tissue-specific RRM2 promoter, a urinary tract tissue-specific uroplakin 2 (UPII) promoter, a uterine tissue-specific lactoferrin promoter, or a cardiac Troponin T (cTnT) promoter. Other exemplary promoters include Beta-actin promoter, hepatitis B virus core promoter, Sandig et al., Gene Ther., 3:1002-9 (1996); alpha-fetoprotein (AFP) promoter, Arbuthnot et al., Hum. Gene Ther., 7:1503-14 (1996)), bone osteocalcin promoter (Stein et al., Mol. Biol. Rep., 24:185-96 (1997)); bone sialoprotein promoter (Chen et al., J. Bone Miner. Res., 11:654-64 (1996)), CD2 promoter (Hansal et al., J. Immunol., 161:1063-8 (1998); immunoglobulin heavy chain promoter; T cell receptor α-chain promoter, neuronal such as neuron-specific enolase (NSE) promoter (Andersen et al., Cell. Mol. Neurobiol., 13:503-15 (1993)), neurofilament light-chain gene promoter (Piccioli et al., Proc. Natl. Acad. Sci. USA, 88:5611-5 (1991)), and the neuron-specific vgf gene promoter (Piccioli et al., Neuron, 15:373-84 (1995)), among others which will be apparent to the skilled artisan.

In some embodiments, one or more bindings sites for one or more of miRNAs are incorporated in a transgene of a rAAV vector, to inhibit the expression of the transgene in one or more tissues of an subject harboring the transgene. The skilled artisan will appreciate that binding sites may be selected to control the expression of a transgene in a tissue specific manner. For example, binding sites for the liver-specific miR-122 may be incorporated into a transgene to inhibit expression of that transgene in the liver. The target sites in the mRNA may be in the 5′ UTR, the 3′ UTR or in the coding region. Typically, the target site is in the 3′ UTR of the mRNA. Furthermore, the transgene may be designed such that multiple miRNAs regulate the mRNA by recognizing the same or multiple sites. The presence of multiple miRNA binding sites may result in the cooperative action of multiple RISCs and provide highly efficient inhibition of expression. The target site sequence may comprise a total of 5-100, 10-60, or more nucleotides. The target site sequence may comprise at least 5 nucleotides of the sequence of a target gene binding site.

The composition of the transgene sequence of the rAAV vector will depend upon the use to which the resulting vector will be put. For example, one type of transgene sequence includes a reporter sequence, which upon expression produces a detectable signal. In another example, the transgene encodes a therapeutic protein or therapeutic functional RNA. In another example, the transgene encodes a protein or functional RNA that is intended to be used for research purposes, e.g., to create a somatic transgenic animal model harboring the transgene, e.g., to study the function of the transgene product. In another example, the transgene encodes a protein or functional RNA that is intended to be used to create an animal model of disease. Appropriate transgene coding sequences will be apparent to the skilled artisan.

Reporter sequences that may be provided in a transgene include, without limitation, DNA sequences encoding β-lactamase, β-galactosidase (LacZ), alkaline phosphatase, thymidine kinase, green fluorescent protein (GFP, EGFP), chloramphenicol acetyltransferase (CAT), luciferase (e.g., Firefly luciferase), and others well known in the art. When associated with regulatory elements which drive their expression, the reporter sequences, provide signals detectable by conventional means, including enzymatic, radiographic, colorimetric, fluorescence or other spectrographic assays, fluorescent activating cell sorting assays and immunological assays, including enzyme linked immunosorbent assay (ELISA), radioimmunoassay (RIA) and immunohistochemistry. For example, where the marker sequence is the LacZ gene, the presence of the vector carrying the signal is detected by assays for-galactosidase activity. Where the transgene is green fluorescent protein or luciferase, the vector carrying the signal may be measured visually by color or light production in a luminometer. Such reporters can, for example, be useful in verifying the tissue-specific targeting capabilities and tissue specific promoter regulatory activity of an rAAV.

In some aspects, the disclosure provides rAAV vectors for use in methods of preventing or treating one or more genetic deficiencies or dysfunctions in a mammal, such as for example, a polypeptide deficiency or polypeptide excess in a mammal, and particularly for treating or reducing the severity or extent of deficiency in a human manifesting one or more of the disorders linked to a deficiency in such polypeptides in cells and tissues. The method involves administration of an rAAV vector that encodes one or more gene products (e.g., therapeutic peptides, polypeptides, siRNAs, microRNAs, antisense nucleotides, etc.) in a pharmaceutically-acceptable carrier to the subject in an amount and for a period of time sufficient to treat the deficiency or disorder in the subject suffering from such a disorder.

Thus, the disclosure embraces the delivery of rAAV vectors encoding one or more peptides, polypeptides, or proteins, which are useful for the treatment or prevention of disease states in a mammalian subject. Exemplary therapeutic proteins include one or more polypeptides selected from the group consisting of growth factors, interleukins, interferons, anti-apoptosis factors, cytokines, anti-diabetic factors, anti-apoptosis agents, coagulation factors, anti-tumor factors. Other non-limiting examples of therapeutic proteins include BDNF, CNTF, CSF, EGF, FGF, G-SCF, GM-CSF, gonadotropin, IFN, IFG-1, M-CSF, NGF, PDGF, PEDF, TGF, VEGF, TGF-B2, TNF, prolactin, somatotropin, XIAP1, IL-1, IL-2, IL-3, IL-4, IL-5, IL-6, IL-7, IL-8, IL-9, IL-10, IL-10 (187A), viral IL-10, IL-11, IL-12, IL-13, IL-14, IL-15, IL-16 IL-17, and IL-18.

The rAAV vectors may comprise a gene to be transferred to a subject to treat a disease associated with reduced expression, lack of expression or dysfunction of the gene. Exemplary genes and associated disease states include, but are not limited to: glucose-6-phosphatase, associated with glycogen storage deficiency type 1A; phosphoenolpyruvate-carboxykinase, associated with Pepck deficiency; galactose-1 phosphate uridyl transferase, associated with galactosemia; phenylalanine hydroxylase, associated with phenylketonuria; branched chain alpha-ketoacid dehydrogenase, associated with Maple syrup urine disease; fumarylacetoacetate hydrolase, associated with tyrosinemia type 1; methylmalonyl-CoA mutase, associated with methylmalonic acidemia; medium chain acyl CoA dehydrogenase, associated with medium chain acetyl CoA deficiency; omithine transcarbamylase, associated with omithine transcarbamylase deficiency; argininosuccinic acid synthetase, associated with citrullinemia; low density lipoprotein receptor protein, associated with familial hypercholesterolemia; UDP-glucouronosyltransferase, associated with Crigler-Najjar disease; adenosine deaminase, associated with severe combined immunodeficiency disease; hypoxanthine guanine phosphoribosyl transferase, associated with Gout and Lesch-Nyan syndrome; biotinidase, associated with biotinidase deficiency; beta-glucocerebrosidase, associated with Gaucher disease; beta-glucuronidase, associated with Sly syndrome; peroxisome membrane protein 70 kDa, associated with Zellweger syndrome; porphobilinogen deaminase, associated with acute intermittent porphyria; alpha-1 antitrypsin for treatment of alpha-1 antitrypsin deficiency (emphysema); erythropoietin for treatment of anemia due to thalassemia or to renal failure; vascular endothelial growth factor, angiopoietin-1, and fibroblast growth factor for the treatment of ischemic diseases; thrombomodulin and tissue factor pathway inhibitor for the treatment of occluded blood vessels as seen in, for example, atherosclerosis, thrombosis, or embolisms; aromatic amino acid decarboxylase (AADC), and tyrosine hydroxylase (TH) for the treatment of Parkinson's disease; the beta adrenergic receptor, anti-sense to, or a mutant form of, phospholamban, the sarcoendoplasmic reticulum adenosine triphosphatase-2 (SERCA2), and the cardiac adenylyl cyclase for the treatment of congestive heart failure; a tumor suppressor gene such as p53 for the treatment of various cancers; a cytokine such as one of the various interleukins for the treatment of inflammatory and immune disorders and cancers; dystrophin or minidystrophin and utrophin or miniutrophin for the treatment of muscular dystrophies; and, insulin for the treatment of diabetes.

The skilled artisan will also realize that in the case of transgenes encoding proteins or polypeptides, mutations that result in conservative amino acid substitutions may be made in a transgene to provide functionally equivalent variants or homologs of a protein or polypeptide. In some aspects the disclosure embraces sequence alterations that result in conservative amino acid substitution of a transgene. In some embodiments, the transgene comprises a gene having a dominant negative mutation. For example, a transgene may express a mutant protein that interacts with the same elements as a wild-type protein, and thereby blocks some aspect of the function of the wild-type protein.

Useful transgene products also include miRNAs. miRNAs and other small interfering nucleic acids regulate gene expression via target RNA transcript cleavage/degradation or translational repression of the target messenger RNA (mRNA). miRNAs are natively expressed, typically as final 19-25 non-translated RNA products. miRNAs exhibit their activity through sequence-specific interactions with the 3′ untranslated regions (UTR) of target mRNAs. These endogenously expressed miRNAs form hairpin precursors that are subsequently processed into a miRNA duplex, and further into a “mature” single stranded miRNA molecule. This mature miRNA guides a multiprotein complex, miRISC, which identifies target sites, e.g., in the 3′ UTR regions, of target mRNAs based upon their complementarity to the mature miRNA.

The following non-limiting list of miRNA genes, and their homologues, are useful as transgenes or as targets for small interfering nucleic acids encoded by transgenes (e.g., miRNA sponges, antisense oligonucleotides, TuD RNAs) in certain embodiments of the methods: hsa-let-7a, hsa-let-7a*, hsa-let-7b, hsa-let-7b*, hsa-let-7c, hsa-let-7c*, hsa-let-7d, hsa-let-7d*, hsa-let-7e, hsa-let-7e*, hsa-let-7f, hsa-let-7f-1*, hsa-let-7f-2*, hsa-let-7g, hsa-let-7g*, hsa-let-7i, hsa-let-7i*, hsa-miR-1, hsa-miR-100, hsa-miR-100*, hsa-miR-101, hsa-miR-101*, hsa-miR-103, hsa-miR-105, hsa-miR-105*, hsa-miR-106a, hsa-miR-106a*, hsa-miR-106b, hsa-miR-106b*, hsa-miR-107, hsa-miR-10a, hsa-miR-10a*, hsa-miR-10b, hsa-miR-10b*, hsa-miR-1178, hsa-miR-1179, hsa-miR-1180, hsa-miR-1181, hsa-miR-1182, hsa-miR-1183, hsa-miR-1184, hsa-miR-1185, hsa-miR-1197, hsa-miR-1200, hsa-miR-1201, hsa-miR-1202, hsa-miR-1203, hsa-miR-1204, hsa-miR-1205, hsa-miR-1206, hsa-miR-1207-3p, hsa-miR-1207-5p, hsa-miR-1208, hsa-miR-122, hsa-miR-122*, hsa-miR-1224-3p, hsa-miR-1224-5p, hsa-miR-1225-3p, hsa-miR-1225-5p, hsa-miR-1226, hsa-miR-1226*, hsa-miR-1227, hsa-miR-1228, hsa-miR-1228*, hsa-miR-1229, hsa-miR-1231, hsa-miR-1233, hsa-miR-1234, hsa-miR-1236, hsa-miR-1237, hsa-miR-1238, hsa-miR-124, hsa-miR-124*, hsa-miR-1243, hsa-miR-1244, hsa-miR-1245, hsa-miR-1246, hsa-miR-1247, hsa-miR-1248, hsa-miR-1249, hsa-miR-1250, hsa-miR-1251, hsa-miR-1252, hsa-miR-1253, hsa-miR-1254, hsa-miR-1255a, hsa-miR-1255b, hsa-miR-1256, hsa-miR-1257, hsa-miR-1258, hsa-miR-1259, hsa-miR-125a-3p, hsa-miR-125a-5p, hsa-miR-125b, hsa-miR-125b-1*, hsa-miR-125b-2*, hsa-miR-126, hsa-miR-126*, hsa-miR-1260, hsa-miR-1261, hsa-miR-1262, hsa-miR-1263, hsa-miR-1264, hsa-miR-1265, hsa-miR-1266, hsa-miR-1267, hsa-miR-1268, hsa-miR-1269, hsa-miR-1270, hsa-miR-1271, hsa-miR-1272, hsa-miR-1273, hsa-miR-127-3p, hsa-miR-1274a, hsa-miR-1274b, hsa-miR-1275, hsa-miR-127-5p, hsa-miR-1276, hsa-miR-1277, hsa-miR-1278, hsa-miR-1279, hsa-miR-128, hsa-miR-1280, hsa-miR-1281, hsa-miR-1282, hsa-miR-1283, hsa-miR-1284, hsa-miR-1285, hsa-miR-1286, hsa-miR-1287, hsa-miR-1288, hsa-miR-1289, hsa-miR-129*, hsa-miR-1290, hsa-miR-1291, hsa-miR-1292, hsa-miR-1293, hsa-miR-129-3p, hsa-miR-1294, hsa-miR-1295, hsa-miR-129-5p, hsa-miR-1296, hsa-miR-1297, hsa-miR-1298, hsa-miR-1299, hsa-miR-1300, hsa-miR-1301, hsa-miR-1302, hsa-miR-1303, hsa-miR-1304, hsa-miR-1305, hsa-miR-1306, hsa-miR-1307, hsa-miR-1308, hsa-miR-130a, hsa-miR-130a*, hsa-miR-130b, hsa-miR-130b*, hsa-miR-132, hsa-miR-132*, hsa-miR-1321, hsa-miR-1322, hsa-miR-1323, hsa-miR-1324, hsa-miR-133a, hsa-miR-133b, hsa-miR-134, hsa-miR-135a, hsa-miR-135a*, hsa-miR-135b, hsa-miR-135b*, hsa-miR-136, hsa-miR-136*, hsa-miR-137, hsa-miR-138, hsa-miR-138-1*, hsa-miR-138-2*, hsa-miR-139-3p, hsa-miR-139-5p, hsa-miR-140-3p, hsa-miR-140-5p, hsa-miR-141, hsa-miR-141*, hsa-miR-142-3p, hsa-miR-142-5p, hsa-miR-143, hsa-miR-143*, hsa-miR-144, hsa-miR-144*, hsa-miR-145, hsa-miR-145*, hsa-miR-146a, hsa-miR-146a*, hsa-miR-146b-3p, hsa-miR-146b-5p, hsa-miR-147, hsa-miR-147b, hsa-miR-148a, hsa-miR-148a*, hsa-miR-148b, hsa-miR-148b*, hsa-miR-149, hsa-miR-149*, hsa-miR-150, hsa-miR-150*, hsa-miR-151-3p, hsa-miR-151-5p, hsa-miR-152, hsa-miR-153, hsa-miR-154, hsa-miR-154*, hsa-miR-155, hsa-miR-155*, hsa-miR-15a, hsa-miR-15a*, hsa-miR-15b, hsa-miR-15b*, hsa-miR-16, hsa-miR-16-1*, hsa-miR-16-2*, hsa-miR-17, hsa-miR-17*, hsa-miR-181a, hsa-miR-181a*, hsa-miR-181a-2*, hsa-miR-181b, hsa-miR-181c, hsa-miR-181c*, hsa-miR-181d, hsa-miR-182, hsa-miR-182*, hsa-miR-1825, hsa-miR-1826, hsa-miR-1827, hsa-miR-183, hsa-miR-183*, hsa-miR-184, hsa-miR-185, hsa-miR-185*, hsa-miR-186, hsa-miR-186*, hsa-miR-187, hsa-miR-187*, hsa-miR-188-3p, hsa-miR-188-5p, hsa-miR-18a, hsa-miR-18a*, hsa-miR-18b, hsa-miR-18b*, hsa-miR-190, hsa-miR-190b, hsa-miR-191, hsa-miR-191*, hsa-miR-192, hsa-miR-192*, hsa-miR-193a-3p, hsa-miR-193a-5p, hsa-miR-193b, hsa-miR-193b*, hsa-miR-194, hsa-miR-194*, hsa-miR-195, hsa-miR-195*, hsa-miR-196a, hsa-miR-196a*, hsa-miR-196b, hsa-miR-197, hsa-miR-198, hsa-miR-199a-3p, hsa-miR-199a-5p, hsa-miR-199b-5p, hsa-miR-19a, hsa-miR-19a*, hsa-miR-19b, hsa-miR-19b-1*, hsa-miR-19b-2*, hsa-miR-200a, hsa-miR-200a*, hsa-miR-200b, hsa-miR-200b*, hsa-miR-200c, hsa-miR-200c*, hsa-miR-202, hsa-miR-202*, hsa-miR-203, hsa-miR-204, hsa-miR-205, hsa-miR-206, hsa-miR-208a, hsa-miR-208b, hsa-miR-20a, hsa-miR-20a*, hsa-miR-20b, hsa-miR-20b*, hsa-miR-21, hsa-miR-21*, hsa-miR-210, hsa-miR-211, hsa-miR-212, hsa-miR-214, hsa-miR-214*, hsa-miR-215, hsa-miR-216a, hsa-miR-216b, hsa-miR-217, hsa-miR-218, hsa-miR-218-1*, hsa-miR-218-2*, hsa-miR-219-1-3p, hsa-miR-219-2-3p, hsa-miR-219-5p, hsa-miR-22, hsa-miR-22*, hsa-miR-220a, hsa-miR-220b, hsa-miR-220c, hsa-miR-221, hsa-miR-221*, hsa-miR-222, hsa-miR-222*, hsa-miR-223, hsa-miR-223*, hsa-miR-224, hsa-miR-23a, hsa-miR-23a*, hsa-miR-23b, hsa-miR-23b*, hsa-miR-24, hsa-miR-24-1*, hsa-miR-24-2*, hsa-miR-25, hsa-miR-25*, hsa-miR-26a, hsa-miR-26a-1*, hsa-miR-26a-2*, hsa-miR-26b, hsa-miR-26b*, hsa-miR-27a, hsa-miR-27a*, hsa-miR-27b, hsa-miR-27b*, hsa-miR-28-3p, hsa-miR-28-5p, hsa-miR-296-3p, hsa-miR-296-5p, hsa-miR-297, hsa-miR-298, hsa-miR-299-3p, hsa-miR-299-5p, hsa-miR-29a, hsa-miR-29a*, hsa-miR-29b, hsa-miR-29b-1*, hsa-miR-29b-2*, hsa-miR-29c, hsa-miR-29c*, hsa-miR-300, hsa-miR-301a, hsa-miR-301b, hsa-miR-302a, hsa-miR-302a*, hsa-miR-302b, hsa-miR-302b*, hsa-miR-302c, hsa-miR-302c*, hsa-miR-302d, hsa-miR-302d*, hsa-miR-302e, hsa-miR-302f, hsa-miR-30a, hsa-miR-30a*, hsa-miR-30b, hsa-miR-30b*, hsa-miR-30c, hsa-miR-30c-1*, hsa-miR-30c-2*, hsa-miR-30d, hsa-miR-30d*, hsa-miR-30e, hsa-miR-30e*, hsa-miR-31, hsa-miR-31*, hsa-miR-32, hsa-miR-32*, hsa-miR-320a, hsa-miR-320b, hsa-miR-320c, hsa-miR-320d, hsa-miR-323-3p, hsa-miR-323-5p, hsa-miR-324-3p, hsa-miR-324-5p, hsa-miR-325, hsa-miR-326, hsa-miR-328, hsa-miR-329, hsa-miR-330-3p, hsa-miR-330-5p, hsa-miR-331-3p, hsa-miR-331-5p, hsa-miR-335, hsa-miR-335*, hsa-miR-337-3p, hsa-miR-337-5p, hsa-miR-338-3p, hsa-miR-338-5p, hsa-miR-339-3p, hsa-miR-339-5p, hsa-miR-33a, hsa-miR-33a*, hsa-miR-33b, hsa-miR-33b*, hsa-miR-340, hsa-miR-340*, hsa-miR-342-3p, hsa-miR-342-5p, hsa-miR-345, hsa-miR-346, hsa-miR-34a, hsa-miR-34a*, hsa-miR-34b, hsa-miR-34b*, hsa-miR-34c-3p, hsa-miR-34c-5p, hsa-miR-361-3p, hsa-miR-361-5p, hsa-miR-362-3p, hsa-miR-362-5p, hsa-miR-363, hsa-miR-363*, hsa-miR-365, hsa-miR-367, hsa-miR-367*, hsa-miR-369-3p, hsa-miR-369-5p, hsa-miR-370, hsa-miR-371-3p, hsa-miR-371-5p, hsa-miR-372, hsa-miR-373, hsa-miR-373*, hsa-miR-374a, hsa-miR-374a*, hsa-miR-374b, hsa-miR-374b*, hsa-miR-375, hsa-miR-376a, hsa-miR-376a*, hsa-miR-376b, hsa-miR-376c, hsa-miR-377, hsa-miR-377*, hsa-miR-378, hsa-miR-378*, hsa-miR-379, hsa-miR-379*, hsa-miR-380, hsa-miR-380*, hsa-miR-381, hsa-miR-382, hsa-miR-383, hsa-miR-384, hsa-miR-409-3p, hsa-miR-409-5p, hsa-miR-410, hsa-miR-411, hsa-miR-411*, hsa-miR-412, hsa-miR-421, hsa-miR-422a, hsa-miR-423-3p, hsa-miR-423-5p, hsa-miR-424, hsa-miR-424*, hsa-miR-425, hsa-miR-425*, hsa-miR-429, hsa-miR-431, hsa-miR-431*, hsa-miR-432, hsa-miR-432*, hsa-miR-433, hsa-miR-448, hsa-miR-449a, hsa-miR-449b, hsa-miR-450a, hsa-miR-450b-3p, hsa-miR-450b-5p, hsa-miR-451, hsa-miR-452, hsa-miR-452*, hsa-miR-453, hsa-miR-454, hsa-miR-454*, hsa-miR-455-3p, hsa-miR-455-5p, hsa-miR-483-3p, hsa-miR-483-5p, hsa-miR-484, hsa-miR-485-3p, hsa-miR-485-5p, hsa-miR-486-3p, hsa-miR-486-5p, hsa-miR-487a, hsa-miR-487b, hsa-miR-488, hsa-miR-488*, hsa-miR-489, hsa-miR-490-3p, hsa-miR-490-5p, hsa-miR-491-3p, hsa-miR-491-5p, hsa-miR-492, hsa-miR-493, hsa-miR-493*, hsa-miR-494, hsa-miR-495, hsa-miR-496, hsa-miR-497, hsa-miR-497*, hsa-miR-498, hsa-miR-499-3p, hsa-miR-499-5p, hsa-miR-500, hsa-miR-500*, hsa-miR-501-3p, hsa-miR-501-5p, hsa-miR-502-3p, hsa-miR-502-5p, hsa-miR-503, hsa-miR-504, hsa-miR-505, hsa-miR-505*, hsa-miR-506, hsa-miR-507, hsa-miR-508-3p, hsa-miR-508-5p, hsa-miR-509-3-5p, hsa-miR-509-3p, hsa-miR-509-5p, hsa-miR-510, hsa-miR-511, hsa-miR-512-3p, hsa-miR-512-5p, hsa-miR-513a-3p, hsa-miR-513a-5p, hsa-miR-513b, hsa-miR-513c, hsa-miR-514, hsa-miR-515-3p, hsa-miR-515-5p, hsa-miR-516a-3p, hsa-miR-516a-5p, hsa-miR-516b, hsa-miR-517*, hsa-miR-517a, hsa-miR-517b, hsa-miR-517c, hsa-miR-518a-3p, hsa-miR-518a-5p, hsa-miR-518b, hsa-miR-518c, hsa-miR-518c*, hsa-miR-518d-3p, hsa-miR-518d-5p, hsa-miR-518e, hsa-miR-518e*, hsa-miR-518f, hsa-miR-518f*, hsa-miR-519a, hsa-miR-519b-3p, hsa-miR-519c-3p, hsa-miR-519d, hsa-miR-519e, hsa-miR-519e*, hsa-miR-520a-3p, hsa-miR-520a-5p, hsa-miR-520b, hsa-miR-520c-3p, hsa-miR-520d-3p, hsa-miR-520d-5p, hsa-miR-520e, hsa-miR-520f, hsa-miR-520g, hsa-miR-520h, hsa-miR-521, hsa-miR-522, hsa-miR-523, hsa-miR-524-3p, hsa-miR-524-5p, hsa-miR-525-3p, hsa-miR-525-5p, hsa-miR-526b, hsa-miR-526b*, hsa-miR-532-3p, hsa-miR-532-5p, hsa-miR-539, hsa-miR-541, hsa-miR-541*, hsa-miR-542-3p, hsa-miR-542-5p, hsa-miR-543, hsa-miR-544, hsa-miR-545, hsa-miR-545*, hsa-miR-548a-3p, hsa-miR-548a-5p, hsa-miR-548b-3p, hsa-miR-548b-5p, hsa-miR-548c-3p, hsa-miR-548c-5p, hsa-miR-548d-3p, hsa-miR-548d-5p, hsa-miR-548e, hsa-miR-548f, hsa-miR-548g, hsa-miR-548h, hsa-miR-548i, hsa-miR-548j, hsa-miR-548k, hsa-miR-5481, hsa-miR-548m, hsa-miR-548n, hsa-miR-5480, hsa-miR-548p, hsa-miR-549, hsa-miR-550, hsa-miR-550*, hsa-miR-551a, hsa-miR-551b, hsa-miR-551b*, hsa-miR-552, hsa-miR-553, hsa-miR-554, hsa-miR-555, hsa-miR-556-3p, hsa-miR-556-5p, hsa-miR-557, hsa-miR-558, hsa-miR-559, hsa-miR-561, hsa-miR-562, hsa-miR-563, hsa-miR-564, hsa-miR-566, hsa-miR-567, hsa-miR-568, hsa-miR-569, hsa-miR-570, hsa-miR-571, hsa-miR-572, hsa-miR-573, hsa-miR-574-3p, hsa-miR-574-5p, hsa-miR-575, hsa-miR-576-3p, hsa-miR-576-5p, hsa-miR-577, hsa-miR-578, hsa-miR-579, hsa-miR-580, hsa-miR-581, hsa-miR-582-3p, hsa-miR-582-5p, hsa-miR-583, hsa-miR-584, hsa-miR-585, hsa-miR-586, hsa-miR-587, hsa-miR-588, hsa-miR-589, hsa-miR-589*, hsa-miR-590-3p, hsa-miR-590-5p, hsa-miR-591, hsa-miR-592, hsa-miR-593, hsa-miR-593*, hsa-miR-595, hsa-miR-596, hsa-miR-597, hsa-miR-598, hsa-miR-599, hsa-miR-600, hsa-miR-601, hsa-miR-602, hsa-miR-603, hsa-miR-604, hsa-miR-605, hsa-miR-606, hsa-miR-607, hsa-miR-608, hsa-miR-609, hsa-miR-610, hsa-miR-611, hsa-miR-612, hsa-miR-613, hsa-miR-614, hsa-miR-615-3p, hsa-miR-615-5p, hsa-miR-616, hsa-miR-616*, hsa-miR-617, hsa-miR-618, hsa-miR-619, hsa-miR-620, hsa-miR-621, hsa-miR-622, hsa-miR-623, hsa-miR-624, hsa-miR-624*, hsa-miR-625, hsa-miR-625*, hsa-miR-626, hsa-miR-627, hsa-miR-628-3p, hsa-miR-628-5p, hsa-miR-629, hsa-miR-629*, hsa-miR-630, hsa-miR-631, hsa-miR-632, hsa-miR-633, hsa-miR-634, hsa-miR-635, hsa-miR-636, hsa-miR-637, hsa-miR-638, hsa-miR-639, hsa-miR-640, hsa-miR-641, hsa-miR-642, hsa-miR-643, hsa-miR-644, hsa-miR-645, hsa-miR-646, hsa-miR-647, hsa-miR-648, hsa-miR-649, hsa-miR-650, hsa-miR-651, hsa-miR-652, hsa-miR-653, hsa-miR-654-3p, hsa-miR-654-5p, hsa-miR-655, hsa-miR-656, hsa-miR-657, hsa-miR-658, hsa-miR-659, hsa-miR-660, hsa-miR-661, hsa-miR-662, hsa-miR-663, hsa-miR-663b, hsa-miR-664, hsa-miR-664*, hsa-miR-665, hsa-miR-668, hsa-miR-671-3p, hsa-miR-671-5p, hsa-miR-675, hsa-miR-7, hsa-miR-708, hsa-miR-708*, hsa-miR-7-1*, hsa-miR-7-2*, hsa-miR-720, hsa-miR-744, hsa-miR-744*, hsa-miR-758, hsa-miR-760, hsa-miR-765, hsa-miR-766, hsa-miR-767-3p, hsa-miR-767-5p, hsa-miR-768-3p, hsa-miR-768-5p, hsa-miR-769-3p, hsa-miR-769-5p, hsa-miR-770-5p, hsa-miR-802, hsa-miR-873, hsa-miR-874, hsa-miR-875-3p, hsa-miR-875-5p, hsa-miR-876-3p, hsa-miR-876-5p, hsa-miR-877, hsa-miR-877*, hsa-miR-885-3p, hsa-miR-885-5p, hsa-miR-886-3p, hsa-miR-886-5p, hsa-miR-887, hsa-miR-888, hsa-miR-888*, hsa-miR-889, hsa-miR-890, hsa-miR-891a, hsa-miR-891b, hsa-miR-892a, hsa-miR-892b, hsa-miR-9, hsa-miR-9*, hsa-miR-920, hsa-miR-921, hsa-miR-922, hsa-miR-923, hsa-miR-924, hsa-miR-92a, hsa-miR-92a-1*, hsa-miR-92a-2*, hsa-miR-92b, hsa-miR-92b*, hsa-miR-93, hsa-miR-93*, hsa-miR-933, hsa-miR-934, hsa-miR-935, hsa-miR-936, hsa-miR-937, hsa-miR-938, hsa-miR-939, hsa-miR-940, hsa-miR-941, hsa-miR-942, hsa-miR-943, hsa-miR-944, hsa-miR-95, hsa-miR-96, hsa-miR-96*, hsa-miR-98, hsa-miR-99a, hsa-miR-99a*, hsa-miR-99b, and hsa-miR-99b*.

miRNA binds to and leads to degradation of the mRNAs it targets and, as a result, inhibits expression of the polypeptides encoded by the mRNAs. Thus, blocking (partially or totally) the presence of the miRNA (e.g., silencing the miRNA) can effectively induce, or restore, expression of a polypeptide whose expression is inhibited (derepress the polypeptide). In one embodiment, derepression of polypeptides encoded by mRNA targets of a miRNA is accomplished by inhibiting the miRNA activity in cells through any one of a variety of methods. For example, blocking the activity of a miRNA can be accomplished by hybridization with a small interfering nucleic acid (e.g., antisense oligonucleotide, miRNA sponge, TuD RNA) that is complementary, or substantially complementary to, the miRNA, thereby blocking interaction of the miRNA with its target mRNA. As used herein, a small interfering nucleic acid that is substantially complementary to a miRNA is one that is capable of hybridizing with a miRNA, and blocking the miRNA's activity. In some embodiments, a small interfering nucleic acid that is substantially complementary to a miRNA is a small interfering nucleic acid that is complementary to the miRNA at all but 1, 2, 3, 4, 5, 6, 7, 8, 9, 10, 11, 12, 13, 14, 15, 16, 17, or 18 bases. In some embodiments, a small interfering nucleic acid sequence that is substantially complementary to a miRNA, or is a small interfering nucleic acid sequence that is complementary to the miRNA with at least one base.

A “miRNA Inhibitor” is an agent that blocks miRNA function, expression and/or processing. For instance, these molecules include, but are not limited to, microRNA specific antisense oligonucleotides, microRNA sponges, tough decoy RNAs (TuD RNAs), and microRNA oligonucleotides (double-stranded, hairpin, short oligonucleotides) that inhibit miRNA interaction with a Drosha complex. MicroRNA inhibitors can be expressed in cells from a transgene of a rAAV vector, as discussed above. MicroRNA sponges specifically inhibit miRNAs through a complementary heptameric seed sequence (Ebert, M. S. Nature Methods, Epub Aug. 12, 2007). In some embodiments, an entire family of miRNAs can be silenced using a single sponge sequence. TuD RNAs achieve efficient and long-term-suppression of specific miRNAs in mammalian cells (See, e.g., Takeshi Haraguchi, et al., Nucleic Acids Research, 2009, Vol. 37, No. 6 e43, the contents of which relating to TuD RNAs are incorporated herein by reference). Other methods for silencing miRNA function (derepression of miRNA targets) in cells will be apparent to one of ordinary skill in the art.

In some embodiments, the cloning capacity of the recombinant RNA vector may limit a desired coding sequence and may require the complete replacement of the virus's 4.8 kilobase genome. Large genes may, therefore, not be suitable for use in a standard recombinant AAV vector, in some cases. The skilled artisan will appreciate that options are available in the art for overcoming a limited coding capacity. For example, the AAV ITRs of two genomes can anneal to form head to tail concatamers, almost doubling the capacity of the vector. Insertion of splice sites allows for the removal of the ITRs from the transcript. Other options for overcoming a limited cloning capacity will be apparent to the skilled artisan.

### Administration

The rAAVs may be delivered to a subject in compositions according to any appropriate methods known in the art. The rAAV, preferably suspended in a physiologically compatible carrier (e.g., in a composition), may be administered to a subject, e.g., host animal, such as a human, mouse, rat, cat, dog, sheep, rabbit, horse, cow, goat, pig, guinea pig, hamster, chicken, turkey, or a non-human primate (e.g., Macaque). In some embodiments a host animal does not include a human.

Delivery of the rAAVs to a mammalian subject may be by, for example, intramuscular injection or by administration into the bloodstream of the mammalian subject. In some embodiments, an rAAV is delivered to the brain of a subject through intrahippocampal, intracranial, or intraparenchymal injections. Administration into the bloodstream may be by injection into a vein, an artery, or any other vascular conduit. In some embodiments, the rAAVs are administered into the bloodstream by way of isolated limb perfusion, a technique well known in the surgical arts, the method essentially enabling the artisan to isolate a limb from the systemic circulation prior to administration of the rAAV virions. A variant of the isolated limb perfusion technique, described in U.S. Pat. No. 6,177,403, can also be employed by the skilled artisan to administer the virions into the vasculature of an isolated limb to potentially enhance transduction into muscle cells or tissue. Moreover, in certain instances, it may be desirable to deliver the virions to the CNS of a subject. By “CNS” is meant all cells and tissue of the brain and spinal cord of a vertebrate. Thus, the term includes, but is not limited to, neuronal cells, glial cells, astrocytes, cereobrospinal fluid (CSF), interstitial spaces, bone, cartilage and the like. Recombinant AAVs may be delivered directly to the CNS or brain by injection into, e.g., the ventricular region, as well as to the striatum (e.g., the caudate nucleus or putamen of the striatum), spinal cord and neuromuscular junction, or cerebellar lobule, with a needle, catheter or related device, using neurosurgical techniques known in the art, such as by stereotactic injection (see, e.g., Stein et al., J Virol 73:3424-3429, 1999; Davidson et al., PNAS 97:3428-3432, 2000; Davidson et al., Nat. Genet. 3:219-223, 1993; and Alisky and Davidson, Hum. Gene Ther. 11:2315-2329, 2000).

The compositions of the disclosure may comprise an rAAV alone, or in combination with one or more other viruses (e.g., a second rAAV encoding having one or more different transgenes). In some embodiments, a composition comprises 1, 2, 3, 4, 5, 6, 7, 8, 9, 10, or more different rAAVs each having one or more different transgenes

Suitable carriers may be readily selected by one of skill in the art in view of the indication for which the rAAV is directed. For example, one suitable carrier includes saline, which may be formulated with a variety of buffering solutions (e.g., phosphate buffered saline). Other exemplary carriers include sterile saline, lactose, sucrose, calcium phosphate, gelatin, dextran, agar, pectin, peanut oil, sesame oil, and water. The selection of the carrier is not a limitation of the present disclosure.

Optionally, the compositions of the disclosure may contain, in addition to the rAAV and carrier(s), other conventional pharmaceutical ingredients, such as preservatives, or chemical stabilizers. Suitable exemplary preservatives include chlorobutanol, potassium sorbate, sorbic acid, sulfur dioxide, propyl gallate, the parabens, ethyl vanillin, glycerin, phenol, and parachlorophenol. Suitable chemical stabilizers include gelatin and albumin.

The rAAVs are administered in sufficient amounts to transfect the cells of a desired tissue and to provide sufficient levels of gene transfer and expression without undue adverse effects. Conventional and pharmaceutically acceptable routes of administration include, but are not limited to, direct delivery to the selected organ (e.g., delivery to the CNS or the brain), oral, inhalation (including intranasal and intratracheal delivery), intraocular, intravenous, intramuscular, subcutaneous, intradermal, intratumoral, intracranial (e.g., intrahippocampal), and other parental routes of administration. Routes of administration may be combined, if desired.

Administration of an rAAV as described herein (e.g., comprising an AAV capsid protein comprising one or more amino acid substitutions corresponding to position P32, K39, N66, A70, G115, S149, V151, P153, A126, T205, N312, R447, T450, Q457, Q461, S492, E499, P521, S525, F533, G546, E548, K556, R585, R588, and/or A593 with reference to amino acid position numbering of a wild-type AAV2 capsid protein (e.g., SEQ ID NO: 1)) to a subject using any one of the routes of administration described herein (e.g., intracranial injection, intrahippocampal injection, intraparenchymal injection, intravenous (IV) injection, or intracerebroventricular (ICV) injection) may result in the rAAV crossing the blood-brain barrier. The ability of the rAAV to cross the blood-brain barrier may be driven by the AAV capsid protein variant of the rAAV.

The dose of rAAV virions required to achieve a particular “therapeutic effect,” e.g., the units of dose in genome copies/per kilogram of body weight (GC/kg), will vary based on several factors including, but not limited to: the route of rAAV virion administration, the level of gene or RNA expression required to achieve a therapeutic effect, the specific disease or disorder being treated, and the stability of the gene or RNA product. One of skill in the art can readily determine a rAAV virion dose range to treat a patient having a particular disease or disorder based on the aforementioned factors, as well as other factors that are well known in the art.

An effective amount of an rAAV is an amount sufficient to target infect an animal or target a desired tissue (e.g., brain tissue). In some embodiments, an effective amount of an rAAV is an amount sufficient to produce a stable somatic transgenic animal model. The effective amount will depend primarily on factors such as the species, age, weight, health of the subject, and the tissue to be targeted, and may thus vary between animals or tissues. For example, an effective amount of the rAAV is generally in the range of from about 1 ml to about 100 ml of solution containing from about 10⁷ to 10¹⁶ genome copies.

In some embodiments the rAAV is administered at a dose of 10⁷, 10⁸, 10⁹, 10¹⁰, 10¹¹, 10¹², 10¹³, 10¹⁴, or 10¹⁵ genome copies per subject. In some embodiments the rAAV is administered at a dose of 10⁷, 10⁸, 10⁹, 10¹⁰, 10¹¹, 10¹², 10¹³, 10¹⁴, or 10¹⁵ genome copies per kg. In some cases, a dosage between about 10¹¹ to 10¹² rAAV genome copies is appropriate. In some embodiments the rAAV is administered to the brain (e.g., by intracranial or intrahippocampal injection) at a dose of about 10⁹ to 10¹⁶ genome copies per subject. In some embodiments, the rAAV is administered to the brain (e.g., by intracranial or intrahippocampal injection) at a dose of about 10⁹, 10¹⁰, 10¹¹, 10¹², 10¹³, 10¹⁴, 10¹⁵, or 10¹⁶ genome copies per subject. In some embodiments, the rAAV is administered to the brain (e.g., by intracranial or intrahippocampal injection) at a dose of 1×10⁹ genome copies per subject. In some embodiments, the rAAV is administered to the brain (e.g., by intracranial or intrahippocampal injection) at a dose of 3.6×10⁹ genome copies per subject. In some cases, stable transgenic animals are produced by multiple doses of an rAAV.

In some embodiments, rAAV compositions are formulated to reduce aggregation of AAV particles in the composition, particularly where high rAAV concentrations are present (e.g., ˜10¹³ gc/mL or more). Methods for reducing aggregation of rAAVs are well-known in the art and, include, for example, addition of surfactants, pH adjustment, salt concentration adjustment, etc. (See, e.g., Wright F R, et al., Molecular Therapy (2005) 12, 171-178, the contents of which are incorporated herein by reference.)

Formulation of pharmaceutically acceptable excipients and carrier solutions is well-known to those of skill in the art, as is the development of suitable dosing and treatment regimens for using the particular compositions described herein in a variety of treatment regimens.

Typically, these formulations may contain at least about 0.1% of the active compound or more, although the percentage of the active ingredient(s) may, of course, be varied and may conveniently be between about 1 or 2% and about 70% or 80% or more of the weight or volume of the total formulation. Naturally, the amount of active compound in each therapeutically useful composition may be prepared is such a way that a suitable dosage will be obtained in any given unit dose of the compound. Factors such as solubility, bioavailability, biological half-life, route of administration, product shelf life, as well as other pharmacological considerations will be contemplated by one skilled in the art of preparing such pharmaceutical formulations, and as such, a variety of dosages and treatment regimens may be desirable.

In certain circumstances it will be desirable to deliver the rAAV-based therapeutic constructs in suitably formulated pharmaceutical compositions disclosed herein either subcutaneously, intraopancreatically, intranasally, parenterally, intravenously, intracranially (e.g., intrahippocampally), intramuscularly, intrathecally, or orally, intraperitoneally, or by inhalation. In some embodiments, the administration modalities as described in U.S. Pat. Nos. 5,543,158; 5,641,515 and 5,399,363 (each specifically incorporated herein by reference in its entirety) may be used to deliver rAAVs. In some embodiments, a preferred mode of administration is by portal vein injection. In some embodiments, a preferred mode of administration is by facial vein injection.

The pharmaceutical forms suitable for injectable use include sterile aqueous solutions or dispersions and sterile powders for the extemporaneous preparation of sterile injectable solutions or dispersions. Dispersions may also be prepared in glycerol, liquid polyethylene glycols, and mixtures thereof and in oils. Under ordinary conditions of storage and use, these preparations contain a preservative to prevent the growth of microorganisms. In many cases the form is sterile and fluid to the extent that easy syringability exists. It must be stable under the conditions of manufacture and storage and must be preserved against the contaminating action of microorganisms, such as bacteria and fungi. The carrier can be a solvent or dispersion medium containing, for example, water, ethanol, polyol (e.g., glycerol, propylene glycol, and liquid polyethylene glycol, and the like), suitable mixtures thereof, and/or vegetable oils. Proper fluidity may be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

For administration of an injectable aqueous solution, for example, the solution may be suitably buffered, if necessary, and the liquid diluent first rendered isotonic with sufficient saline or glucose. These particular aqueous solutions are especially suitable for intravenous, intramuscular, subcutaneous and intraperitoneal administration. In this connection, a sterile aqueous medium that can be employed will be known to those of skill in the art. For example, one dosage may be dissolved in 1 mL of isotonic NaCl solution and either added to 1000 mL of hypodermoclysis fluid or injected at the proposed site of infusion, (see for example, “Remington's Pharmaceutical Sciences” 15th Edition, pages 1035-1038 and 1570-1580). Some variation in dosage will necessarily occur depending on the condition of the host. The person responsible for administration will, in any event, determine the appropriate dose for the individual host.

Sterile injectable solutions are prepared by incorporating the active rAAV in the required amount in the appropriate solvent with various other ingredients enumerated herein, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof.

The rAAV compositions disclosed herein may also be formulated in a neutral or salt form. Pharmaceutically acceptable salts, include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like. Upon formulation, solutions will be administered in a manner compatible with the dosage formulation and in such amount as is therapeutically effective. The formulations are easily administered in a variety of dosage forms such as injectable solutions, drug-release capsules, and the like.

As used herein, “carrier” includes any and all solvents, dispersion media, vehicles, coatings, diluents, antibacterial and antifungal agents, isotonic and absorption delaying agents, buffers, carrier solutions, suspensions, colloids, and the like. The use of such media and agents for pharmaceutical active substances is well known in the art. Supplementary active ingredients can also be incorporated into the compositions. The phrase “pharmaceutically acceptable” refers to molecular entities and compositions that do not produce an allergic or similar untoward reaction when administered to a host.

Delivery vehicles such as liposomes, nanocapsules, microparticles, microspheres, lipid particles, vesicles, and the like, may be used for the introduction of the compositions of the present disclosure into suitable host cells. In particular, the rAAV vector delivered transgenes may be formulated for delivery either encapsulated in a lipid particle, a liposome, a vesicle, a nanosphere, or a nanoparticle or the like.

Such formulations may be preferred for the introduction of pharmaceutically acceptable formulations of the nucleic acids or the rAAV constructs disclosed herein. The formation and use of liposomes is generally known to those of skill in the art. Recently, liposomes were developed with improved serum stability and circulation half-times (U.S. Pat. No. 5,741,516). Further, various methods of liposome and liposome like preparations as potential drug carriers have been described (U.S. Pat. Nos. 5,567,434; 5,552,157; 5,565,213; 5,738,868 and 5,795,587).

Liposomes have been used successfully with a number of cell types that are normally resistant to transfection by other procedures. In addition, liposomes are free of the DNA length constraints that are typical of viral-based delivery systems. Liposomes have been used effectively to introduce genes, drugs, radiotherapeutic agents, viruses, transcription factors and allosteric effectors into a variety of cultured cell lines and animals. In addition, several successful clinical trials examining the effectiveness of liposome-mediated drug delivery have been completed.

Liposomes are formed from phospholipids that are dispersed in an aqueous medium and spontaneously form multilamellar concentric bilayer vesicles (also termed multilamellar vesicles (MLVs). MLVs generally have diameters of from 25 nm to 4 μm. Sonication of MLVs results in the formation of small unilamellar vesicles (SUVs) with diameters in the range of 200 to 500.ANG., containing an aqueous solution in the core.

Alternatively, nanocapsule formulations of the rAAV may be used. Nanocapsules can generally entrap substances in a stable and reproducible way. To avoid side effects due to intracellular polymeric overloading, such ultrafine particles (sized around 0.1 μm) should be designed using polymers able to be degraded in vivo. Biodegradable polyalkyl-cyanoacrylate nanoparticles that meet these requirements are contemplated for use.

In addition to the methods of delivery described above, the following techniques are also contemplated as alternative methods of delivering the rAAV compositions to a host. Sonophoresis (i.e., ultrasound) has been used and described in U.S. Pat. No. 5,656,016 as a device for enhancing the rate and efficacy of drug permeation into and through the circulatory system. Other drug delivery alternatives contemplated are intraosseous injection (U.S. Pat. No. 5,779,708), microchip devices (U.S. Pat. No. 5,797,898), ophthalmic formulations (Bourlais et al., 1998), transdermal matrices (U.S. Pat. Nos. 5,770,219 and 5,783,208) and feedback-controlled delivery (U.S. Pat. No. 5,697,899).

TABLE 2
Selected amino acid sequences

| | | SEQ ID |
|---|---|---|
| Capsid | Sequence (N-term to C-term) | NO |
| AAV2 | MAADGYLPDWLEDTLSEGIRQWWKLKPGPPPPKPAERHKDDSRG | 1 |
| | LVLPGYKYLGPFNGLDKGEPVNEADAAALEHDKAYDRQLDSGD | |
| | NPYLKYNHADAEFQERLKEDTSFGGNLGRAVFQAKKRVLEPLGL | |
| | VEEPVKTAPGKKRPVEHSPVEPDSSSGTGKAGQQPARKRLNFGQT | |
| | GDADSVPDPQPLGQPPAAPSGLGTNTMATGSGAPMADNNEGAD | |
| | GVGNSSGNWHCDSTWMGDRVITTSTRTWALPTYNNHLYKQISSQ | |
| | SGASNDNHYFGYSTPWGYFDFNRFHCHFSPRDWQRLINNNWGFR | |
| | PKRLNFKLFNIQVKEVTQNDGTTTIANNLTSTVQVFTDSEYQLPY | |
| | VLGSAHQGCLPPFPADVFMVPQYGYLTLNNGSQAVGRSSFYCLE | |
| | YFPSQMLRTGNNFTFSYTFEDVPFHSSYAHSQSLDRLMNPLIDQY | |
| | LYYLSRTNTPSGTTTQSRLQFSQAGASDIRDQSRNWLPGPCYRQQ | |
| | RVSKTSADNNNSEYSWTGATKYHLNGRDSLVNPGPAMASHKDD | |
| | EEKFFPQSGVLIFGKQGSEKTNVDIEKVMITDEEEIRTTNPVATEQ | |
| | YGSVSTNLQRGNRQAATADVNTQGVLPGMVWQDRDVYLQGPI | |
| | WAKIPHTDGHFHPSPLMGGFGLKHPPPQILIKNTPVPANPSTTFSA | |
| | AKFASFITQYSTGQVSVEIEWELQKENSKRWNPEIQYTSNYNKSV | |
| | NVDFTVDTNGVYSEPRPIGTRYLTRNL | |
| AAV2.V46 | MAADGYLPDWLEDTLSEGIRQWWKLKPGPPPPKPAERHQDDSRG | 2 |
| | LVLPGYKYLGPFNGLDKGEPVSEADVAALEHDKAYDRQLDSGD | |
| | NPYLKYNHADAEFQERLKEDTSFGGNLGRAVFQAKKRVLEPLGL | |
| | VEEPVKTAPGKKRPVEHSPAEPDSSSGTGKAGQQPARKRLNFGQT | |
| | GDADSVPDPQPLGQPPAAPSGLGTNTMATGSGAPMADNNEGAD | |
| | GVGNSSGNWHCDSTWMGDRVITTSTRTWALPTYNNHLYKQISSQ | |
| | SGASNDNHYFGYSTPWGYFDFNRFHCHFSPRDWQRLINNNWGFR | |
| | PKRLNFKLFNIQVKEVTQNDGTTTIANNLTSTVQVFTDSEYQLPY | |
| | VLGSAHQGCLPPFPADVFMVPQYGYLTLNNGSQAVGRSSFYCLE | |
| | YFPSQMLRTGNNFTFSYTFEDVPFHSSYAHSQSLDRLMNPLIDQY | |
| | LYYLSKTNAPSGTTTMSRLQFSQAGASDIRDQSRNWLPGPCYRQQ | |
| | RVSKTAADNNNSDYSWTGATKYHLNGRDSLVNPGPAMASHKDD | |
| | EEKYFPQSGVLIFGKQDSGKTNVDIEKVMITDEEEIRTTNPVATEQ | |
| | YGSVSTNLQSGNTQAATTDVNTQGVLPGMVWQDRDVYLQGPIW | |
| | AKIPHTDGHFHPSPLMGGFGLKHPPPQILIKNTPVPANPSTTFSAAK | |
| | FASFITQYSTGQVSVEIEWELQKENSKRWNPEIQYTSNYNKSVNV | |
| | DFTVDTNGVYSEPRPIGTRYLTRNL | |
| AAV2.V56 | MAADGYLPDWLEDTLSEGIRQWWKLKPGPPPPKPAERHQDDSRG | 3 |
| | LVLPGYKYLGPFNGLDKGEPVNEADAAALEHDKAYDRQLDSGD | |
| | NPYLKYNHADAEFQERLKEDTSFGGNLGRAVFQAKKRVLEPLGL | |
| | VEEPVKTAPGKKRPVEHFPAEPDSSSGTGKAGQQPARKRLNFGQT | |
| | GDADSVPDPQPLGQPPAAPSGLGTNTMATGSGAPMADNNEGAD | |
| | GVGNSSGNWHCDSTWMGDRVITTSTRTWALPTYNNHLYKQISSQ | |
| | SGASNDNHYFGYSTPWGYFDFNRFHCHFSPRDWQRLINNNWGFR | |
| | PKRLNFKLFNIQVKEVTQNDGTTTIANNLTSTVQVFTDSEYQLPY | |
| | VLGSAHQGCLPPFPADVFMVPQYGYLTLNNGSQAVGRSSFYCLE | |
| | YFPSQMLRTGNNFTFSYTFEDVPFHSSYAHSQSLDRLMNPLIDQY | |
| | LYYLSKTNAPSGTTTMSRLQFSQAGASDIRDQSRNWLPGPCYRQQ | |
| | RVSKTAADNNNSDYSWTGATKYHLNGRDSLVNPGPAMASHKDD | |
| | EEKYFPQSGVLIFGKQDSGKTNVDIEKVMITDEEEIRTTNPVATEQ | |
| | YGSVSTNLQSGNTQAATTDVNTQGVLPGMVWQDRDVYLQGPIW | |
| | AKIPHTDGHFHPSPLMGGFGLKHPPPQILIKNTPVPANPSTTFSAAK | |
| | FASFITQYSTGQVSVEIEWELQKENSKRWNPEIQYTSNYNKSVNV | |
| | DFTVDTNGVYSEPRPIGTRYLTRNL | |
| AAV2.V67 | MAADGYLPDWLEDTLSEGIRQWWKLKPGPPPPKPAERHQDDSRG | 4 |
| | LVLPGYKYLGPFNGLDKGEPVNEADVAALEHDKAYDRQLDSGD | |
| | NPYLKYNHADAEFQERLKEDTSFGGNLGRAVFQAKKRVLEPLGL | |
| | VEEPVKTAPGKKRPVEHSPAEPDSSSGTGKAGQQPARKRLNFGQT | |
| | GDADSVPDPQPLGQPPAAPSGLGTNTMATGSGAPMADNNEGAD | |
| | GVGNSSGNWHCDSTWMGDRVITTSTRTWALPTYNNHLYKQISSQ | |
| | SGASNDNHYFGYSTPWGYFDFNRFHCHFSPRDWQRLINNNWGFR | |
| | PKRLNFKLFNIQVKEVTQNDGTTTIANNLTSTVQVFTDSEYQLPY | |
| | VLGSAHQGCLPPFPADVFMVPQYGYLTLNNGSQAVGRSSFYCLE | |
| | YFPSQMLRTGNNFTFSYTFEDVPFHSSYAHSQSLDRLMNPLIDQY | |
| | LYYLSKTNAPSGTTTMSRLQFSQAGASDIRDQSRNWLPGPCYRQQ | |
| | RVSKTAADNNNSDYSWTGATKYHLNGRDSLVNPGPAMASHKDD | |
| | EEKYFPQSGVLIFGKQDSGKTNVDIEKVMITDEEEIRTTNPVATEQ | |
| | YGSVSTNLQSGNTQAATTDVNTQGVLPGMVWQDRDVYLQGPIW | |
| | AKIPHTDGHFHPSPLMGGFGLKHPPPQILIKNTPVPANPSTTFSAAK | |
| | FASFITQYSTGQVSVEIEWELQKENSKRWNPEIQYTSNYNKSVNV | |
| | DFTVDTNGVYSEPRPIGTRYLTRNL | |
| AAV2.V81 | MAADGYLPDWLEDTLSEGIRQWWKLKPGPPPLKPAERHQDDSR | 5 |
| | GLVLPGYKYLGPFNGLDKGEPVNEADAAALEHDKAYDRQLDSG | |
| | DNPYLKYNHADAEFQERLKEDTSFGGNLGRAVFQAKKRVLEPLG | |
| | LVEEPVKTAPGKKRPVEHSPAEPDSSSGTGKAGQQPARKRLNFGQ | |
| | TGDADSVPDPQPLGQPPAAPSGLGTNTMAAGSGAPMADNNEGA | |
| | DGVGNSSGNWHCDSTWMGDRVITTSTRTWALPTYNNHLYKQISS | |
| | QSGASNDNHYFGYSTPWGYFDFNRFHCHFSPRDWQRLINNNWGF | |
| | RPKRLNFKLFNIQVKEVTQNDGTTTIANNLTSTVQVFTDSEYQLP | |
| | YVLGSAHQGCLPPFPADVFMVPQYGYLTLNNGSQAVGRSSFYCL | |
| | EYFPSQMLRTGNNFTFSYTFEDVPFHSSYAHSQSLDRLMNPLIDQ | |
| | YLYYLSKTNAPSGTTTMSRLQFSQAGASDIRDQSRNWLPGPCYRQ | |
| | QRVSKTAADNNNSDYSWTGATKYHLNGRDSLVNPGPAMASHKD | |
| | DEEKYFPQSGVLIFGKQDSGKTNVDIEKVMITDEEEIRTTNPVATE | |
| | QYGSVSTNLQSGNTQAATTDVNTQGVLPGMVWQDRDVYLQGPI | |
| | WAKIPHTDGHFHPSPLMGGFGLKHPPPQILIKNTPVPANPSTTFSA | |
| | AKFASFITQYSTGQVSVEIEWELQKENSKRWNPEIQYTSNYNKSV | |
| | NVDFTVDTNGVYSEPRPIGTRYLTRNL | |
| AAV2.V224 | MAADGYLPDWLEDTLSEGIRQWWKLKPGPPPPKPAERHKDDSRG | 6 |
| | LVLPGYKYLGPFNGLDKGEPVNEADAAALEHDKAYDRQLDSGD | |
| | NPYLKYNHADAEFQERLKEDTSFGGNLARAVFQAKKRVLEPLGL | |
| | VEEPVKTAPGKKRPVEHSPAEPDSSSGTGKSGQQPARKRLNFGQT | |
| | GDADSVPDPQPLGQPPAAPSGLGTNTMASGSGAPMADNNEGAD | |
| | GVGNSSGNWHCDSTWMGDRVITTSTRTWALPTYNNHLYKQISSQ | |
| | SGASNDNHYFGYSTPWGYFDFNRFHCHFSPRDWQRLINNNWGFR | |
| | PKRLNFKLFNIQVKEVTQNDGTTTIANNLTSTVQVFTDSEYQLPY | |
| | VLGSAHQGCLPPFPADVFMVPQYGYLTLNNGSQAVGRSSFYCLE | |
| | YFPSQMLRTGNNFTFSYTFEDVPFHSSYAHSQSLDRLMNPLIDQY | |
| | LYYLSRTNTPSGTTTQSRLRFSQAGASDIRDQSRNWLPGPCYRQQ | |
| | RVSKTAADNNNSDYSWTGATKYHLNGRDSLVNPGTAMASHKDD | |
| | EEKYFPQSGVLIFGKQDSEKTNVDIERVMITDEEEIRTTNPVATEQ | |
| | YGSVSTNLQSGNTQAATSDVNTQGVLPGMVWQDRDVYLQGPIW | |
| | AKIPHTDGHFHPSPLMGGFGLKHPPPQILIKNTPVPANPSTTFSAAK | |
| | FASFITQYSTGQVSVEIEWELQKENSKRWNPEIQYTSNYNKSVNV | |
| | DFTVDTNGVYSEPRPIGTRYLTRNL | |
| AAV2.v326 | MAADGYLPDWLEDTLSEGIRQWWKLKPGPPPPKPAERHKDDSRG | 7 |
| | LVLPGYKYLGPFNGLDKGEPVNEADAAALEHDKAYDRQLDSGD | |
| | NPYLKYNHADAEFQERLKEDTSFGGNLGRAVFQAKKRVLEPLGL | |
| | VEEPVKTAPGKKRPVEHSPVEPDSSSGTGKAGQQPARKRLNFGQT | |
| | GDADSVPDPQPLGQPPAAPSGLGTNTMATGSGAPMADNNEGAD | |
| | GVGNSSGNWHCDSTWMGDRVITTSTRTWALPTYNNHLYKQISSQ | |
| | SGASNDNHYFGYSTPWGYFDFNRFHCHFSPRDWQRLINNNWGFR | |
| | PKRLSFKLFNIQVKEVTQNDGTTTIANNLTSTVQVFTDSEYQLPYV | |
| | LGSAHQGCLPPFPADVFMVPQYGYLTLNNGSQAVGRSSFYCLEY | |
| | FPSQMLRTGNNFTFSYTFEDVPFHSSYAHSQSLDRLMNPLIDQYL | |
| | YYLSKTNAPSGTTTMSRLQFSQAGASDIRDQSRNWLPGPCYRQQ | |
| | RVSKTAADNNNSDYSWTGATKYHLNGRDSLVNPGPAMANHKD | |
| | DEEKYFPQSGVLIFGKQGSNKTNVDIEKVMITDEEEIRTTNPVATE | |
| | QYGSVSTNLQSGNTQAATSDVNTQGVLPGMVWQDRDVYLQGPI | |
| | WAKIPHTDGHFHPSPLMGGFGLKHPPPQILIKNTPVPANPSTTFSA | |
| | AKFASFITQYSTGQVSVEIEWELQKENSKRWNPEIQYTSNYNKSV | |
| | NVDFTVDTNGVYSEPRPIGTRYLTRNL | |
| AAV2.V358 | MAADGYLPDWLEDTLSEGIRQWWKLKPGPPPPKPAERHKDDSRG | 8 |
| | LVLPGYKYLGPFNGLDKGEPVNEADAAALEHDKAYDRQLDSGD | |
| | NPYLKYNHADAEFQERLKEDTSFGGNLGRAVFQAKKRVLEPLGL | |
| | VEEPVKTAPGKKRPVEHSPVESDSSSGTGKAGQQPARKRLNFGQT | |
| | GDADSVPDPQPLGQPPAAPSGLGTNTMATGSGAPMADNNEGAD | |
| | GVGNSSGNWHCDSTWMGDRVITTSTRTWALPTYNNHLYKQISSQ | |
| | SGASNDNHYFGYSTPWGYFDFNRFHCHFSPRDWQRLINNNWGFR | |
| | PKRLSFKLFNIQVKEVTQNDGTTTIANNLTSTVQVFTDSEYQLPYV | |
| | LGSAHQGCLPPFPADVFMVPQYGYLTLNNGSQAVGRSSFYCLEY | |
| | FPSQMLRTGNNFTFSYTFEDVPFHSSYAHSQSLDRLMNPLIDQYL | |
| | YYLSKTNAPSGTTTMSRLQFSQAGASDIRDQSRNWLPGPCYRQQ | |
| | RVSKTAADNNNSDYSWTGATKYHLNGRDSLVNPGPAMASHKDD | |
| | EEKYFPQSGVLIFGKQGSNKTNVDIEKVMITDEEEIRTTNPVATEQ | |
| | YGSVSTNLQSGNTQAATSDVNTQGVLPGMVWQDRDVYLQGPIW | |
| | AKIPHTDGHFHPSPLMGGFGLKHPPPQILIKNTPVPANPSTTFSAAK | |
| | FASFITQYSTGQVSVEIEWELQKENSKRWNPEIQYTSNYNKSVNV | |
| | DFTVDTNGVYSEPRPIGTRYLTRNL | |

TABLE 3
Selected nucleic acid sequences

| | | SEQ ID |
|---|---|---|
| Capsid | Sequence (5′ to 3′) | NO |
| AAV2 | Atggcggcggatggctatctgccggattggctggaagataccctgagcgaaggcattcgc | 9 |
| | cagtggtggaaactgaaaccgggcccgccgccgccgaaaccggcggaacgccataaagat | |
| | gatagccgcggcctggtgctgccgggctataaatatctgggcccgtttaacggcctggat | |
| | aaaggcgaaccggtgaacgaagcggatgcggcggcgctggaacatgataaagcgtatgat | |
| | cgccagctggatagcggcgataacccgtatctgaaatataaccatgcggatgcggaattt | |
| | caggaacgcctgaaagaagataccagctttggcggcaacctgggccgcgcggtgtttcag | |
| | gcgaaaaaacgcgtgctggaaccgctgggcctggtggaagaaccggtgaaaaccgcgccg | |
| | ggcaaaaaacgcccggtggaacatagcccggtggaaccggatagcagcagcggcaccggc | |
| | aaagcgggccagcagccggcgcgcaaacgcctgaactttggccagaccggcgatgcggat | |
| | agcgtgccggatccgcagccgctgggccagccgccggcggcgccgagcggcctgggcacc | |
| | aacaccatggcgaccggcagcggcgcgccgatggcggataacaacgaaggcgcggatggc | |
| | gtgggcaacagcagcggcaactggcattgcgatagcacctggatgggcgatcgcgtgatt | |
| | accaccagcacccgcacctgggcgctgccgacctataacaaccatctgtataaacagatt | |
| | agcagccagagcggcgcgagcaacgataaccattattttggctatagcaccccgtggggc | |
| | tattttgattttaaccgctttcattgccattttagcccgcgcgattggcagcgcctgatt | |
| | aacaacaactggggctttcgcccgaaacgcctgaactttaaactgtttaacattcaggtg | |
| | aaagaagtgacccagaacgatggcaccaccaccattgcgaacaacctgaccagcaccgtg | |
| | caggtgtttaccgatagcgaatatcagctgccgtatgtgctgggcagcgcgcatcagggc | |
| | tgcctgccgccgtttccggcggatgtgtttatggtgccgcagtatggctatctgaccctg | |
| | aacaacggcagccaggcggtgggccgcagcagcttttattgcctggaatattttccgagc | |
| | cagatgctgcgcaccggcaacaactttacctttagctatacctttgaagatgtgccgttt | |
| | catagcagctatgcgcatagccagagcctggatcgcctgatgaacccgctgattgatcag | |
| | tatctgtattatctgagccgcaccaacaccccgagcggcaccaccacccagagccgcctg | |
| | cagtttagccaggcgggcgcgagcgatattcgcgatcagagccgcaactggctgccgggc | |
| | ccgtgctatcgccagcagcgcgtgagcaaaaccagcgcggataacaacaacagcgaatat | |
| | agctggaccggcgcgaccaaatatcatctgaacggccgcgatagcctggtgaacccgggc | |
| | ccggcgatggcgagccataaagatgatgaagaaaaattttttccgcagagcggcgtgctg | |
| | atttttggcaaacagggcagcgaaaaaaccaacgtggatattgaaaaagtgatgattacc | |
| | gatgaagaagaaattcgcaccaccaacccggtggcgaccgaacagtatggcagcgtgagc | |
| | accaacctgcagcgcggcaaccgccaggcggcgaccgcggatgtgaacacccagggcgtg | |
| | ctgccgggcatggtgtggcaggatcgcgatgtgtatctgcagggcccgatttgggcgaaa | |
| | attccgcataccgatggccattttcatccgagcccgctgatgggcggctttggcctgaaa | |
| | catccgccgccgcagattctgattaaaaacaccccggtgccggcgaacccgagcaccacc | |
| | tttagcgcggcgaaatttgcgagctttattacccagtatagcaccggccaggtgagcgtg | |
| | gaaattgaatgggaactgcagaaagaaaacagcaaacgctggaacccggaaattcagtat | |
| | accagcaactataacaaaagcgtgaacgtggattttaccgtggataccaacggcgtgtat | |
| | agcgaaccgcgcccgattggcacccgctatctgacccgcaacctg | |
| AAV2.V46 | atggcggcggatggctatctgccggattggctggaagataccctgagcgaaggcattcgc | 10 |
| | cagtggtggaaactgaaaccgggcccgccgccgccgaaaccggcggaacgccatcaggat | |
| | gatagccgcggcctggtgctgccgggctataaatatctgggcccgtttaacggcctggat | |
| | aaaggcgaaccggtgagcgaagcggatgtggcggcgctggaacatgataaagcgtatgat | |
| | cgccagctggatagcggcgataacccgtatctgaaatataaccatgcggatgcggaattt | |
| | caggaacgcctgaaagaagataccagctttggcggcaacctgggccgcgcggtgtttcag | |
| | gcgaaaaaacgcgtgctggaaccgctgggcctggtggaagaaccggtgaaaaccgcgccg | |
| | ggcaaaaaacgcccggtggaacatagcccggcggaaccggatagcagcagcggcaccggc | |
| | aaagcgggccagcagccggcgcgcaaacgcctgaactttggccagaccggcgatgcggat | |
| | agcgtgccggatccgcagccgctgggccagccgccggcggcgccgagcggcctgggcacc | |
| | aacaccatggcgaccggcagcggcgcgccgatggcggataacaacgaaggcgcggatggc | |
| | gtgggcaacagcagcggcaactggcattgcgatagcacctggatgggcgatcgcgtgatt | |
| | accaccagcacccgcacctgggcgctgccgacctataacaaccatctgtataaacagatt | |
| | agcagccagagcggcgcgagcaacgataaccattattttggctatagcaccccgtggggc | |
| | tattttgattttaaccgctttcattgccattttagcccgcgcgattggcagcgcctgatt | |
| | aacaacaactggggctttcgcccgaaacgcctgaactttaaactgtttaacattcaggtg | |
| | aaagaagtgacccagaacgatggcaccaccaccattgcgaacaacctgaccagcaccgtg | |
| | caggtgtttaccgatagcgaatatcagctgccgtatgtgctgggcagcgcgcatcagggc | |
| | tgcctgccgccgtttccggcggatgtgtttatggtgccgcagtatggctatctgaccctg | |
| | aacaacggcagccaggcggtgggccgcagcagcttttattgcctggaatattttccgagc | |
| | cagatgctgcgcaccggcaacaactttacctttagctatacctttgaagatgtgccgttt | |
| | catagcagctatgcgcatagccagagcctggatcgcctgatgaacccgctgattgatcag | |
| | tatctgtattatctgagcaaaaccaacgcgccgagcggcaccaccaccatgagccgcctg | |
| | cagtttagccaggcgggcgcgagcgatattcgcgatcagagccgcaactggctgccgggc | |
| | ccgtgctatcgccagcagcgcgtgagcaaaaccgcggcggataacaacaacagcgattat | |
| | agctggaccggcgcgaccaaatatcatctgaacggccgcgatagcctggtgaacccgggc | |
| | ccggcgatggcgagccataaagatgatgaagaaaaatattttccgcagagcggcgtgctg | |
| | atttttggcaaacaggatagcggcaaaaccaacgtggatattgaaaaagtgatgattacc | |
| | gatgaagaagaaattcgcaccaccaacccggtggcgaccgaacagtatggcagcgtgagc | |
| | accaacctgcagagcggcaacacccaggcggcgaccaccgatgtgaacacccagggcgtg | |
| | ctgccgggcatggtgtggcaggatcgcgatgtgtatctgcagggcccgatttgggcgaaa | |
| | attccgcataccgatggccattttcatccgagcccgctgatgggcggctttggcctgaaa | |
| | catccgccgccgcagattctgattaaaaacaccccggtgccggcgaacccgagcaccacc | |
| | tttagcgcggcgaaatttgcgagctttattacccagtatagcaccggccaggtgagcgtg | |
| | gaaattgaatgggaactgcagaaagaaaacagcaaacgctggaacccggaaattcagtat | |
| | accagcaactataacaaaagcgtgaacgtggattttaccgtggataccaacggcgtgtat | |
| | agcgaaccgcgcccgattggcacccgctatctgacccgcaacctg | |
| AAV2.V56 | atggcggcggatggctatctgccggattggctggaagataccctgagcgaaggcattcgc | 11 |
| | cagtggtggaaactgaaaccgggcccgccgccgccgaaaccggcggaacgccatcaggat | |
| | gatagccgcggcctggtgctgccgggctataaatatctgggcccgtttaacggcctggat | |
| | aaaggcgaaccggtgaacgaagcggatgcggcggcgctggaacatgataaagcgtatgat | |
| | cgccagctggatagcggcgataacccgtatctgaaatataaccatgcggatgcggaattt | |
| | caggaacgcctgaaagaagataccagctttggcggcaacctgggccgcgcggtgtttcag | |
| | gcgaaaaaacgcgtgctggaaccgctgggcctggtggaagaaccggtgaaaaccgcgccg | |
| | ggcaaaaaacgcccggtggaacattttccggcggaaccggatagcagcagcggcaccggc | |
| | aaagcgggccagcagccggcgcgcaaacgcctgaactttggccagaccggcgatgcggat | |
| | agcgtgccggatccgcagccgctgggccagccgccggcggcgccgagcggcctgggcacc | |
| | aacaccatggcgaccggcagcggcgcgccgatggcggataacaacgaaggcgcggatggc | |
| | gtgggcaacagcagcggcaactggcattgcgatagcacctggatgggcgatcgcgtgatt | |
| | accaccagcacccgcacctgggcgctgccgacctataacaaccatctgtataaacagatt | |
| | agcagccagagcggcgcgagcaacgataaccattattttggctatagcaccccgtggggc | |
| | tattttgattttaaccgctttcattgccattttagcccgcgcgattggcagcgcctgatt | |
| | aacaacaactggggctttcgcccgaaacgcctgaactttaaactgtttaacattcaggtg | |
| | aaagaagtgacccagaacgatggcaccaccaccattgcgaacaacctgaccagcaccgtg | |
| | caggtgtttaccgatagcgaatatcagctgccgtatgtgctgggcagcgcgcatcagggc | |
| | tgcctgccgccgtttccggcggatgtgtttatggtgccgcagtatggctatctgaccctg | |
| | aacaacggcagccaggcggtgggccgcagcagcttttattgcctggaatattttccgagc | |
| | cagatgctgcgcaccggcaacaactttacctttagctatacctttgaagatgtgccgttt | |
| | catagcagctatgcgcatagccagagcctggatcgcctgatgaacccgctgattgatcag | |
| | tatctgtattatctgagcaaaaccaacgcgccgagcggcaccaccaccatgagccgcctg | |
| | cagtttagccaggcgggcgcgagcgatattcgcgatcagagccgcaactggctgccgggc | |
| | ccgtgctatcgccagcagcgcgtgagcaaaaccgcggcggataacaacaacagcgattat | |
| | agctggaccggcgcgaccaaatatcatctgaacggccgcgatagcctggtgaacccgggc | |
| | ccggcgatggcgagccataaagatgatgaagaaaaatattttccgcagagcggcgtgctg | |
| | atttttggcaaacaggatagcggcaaaaccaacgtggatattgaaaaagtgatgattacc | |
| | gatgaagaagaaattcgcaccaccaacccggtggcgaccgaacagtatggcagcgtgagc | |
| | accaacctgcagagcggcaacacccaggcggcgaccaccgatgtgaacacccagggcgtg | |
| | ctgccgggcatggtgtggcaggatcgcgatgtgtatctgcagggcccgatttgggcgaaa | |
| | attccgcataccgatggccattttcatccgagcccgctgatgggcggctttggcctgaaa | |
| | catccgccgccgcagattctgattaaaaacaccccggtgccggcgaacccgagcaccacc | |
| | tttagcgcggcgaaatttgcgagctttattacccagtatagcaccggccaggtgagcgtg | |
| | gaaattgaatgggaactgcagaaagaaaacagcaaacgctggaacccggaaattcagtat | |
| | accagcaactataacaaaagcgtgaacgtggattttaccgtggataccaacggcgtgtat | |
| | agcgaaccgcgcccgattggcacccgctatctgacccgcaacctg | |
| AAV2.V67 | atggcggcggatggctatctgccggattggctggaagataccctgagcgaaggcattcgc | 12 |
| | cagtggtggaaactgaaaccgggcccgccgccgccgaaaccggcggaacgccatcaggat | |
| | gatagccgcggcctggtgctgccgggctataaatatctgggcccgtttaacggcctggat | |
| | aaaggcgaaccggtgaacgaagcggatgtggcggcgctggaacatgataaagcgtatgat | |
| | cgccagctggatagcggcgataacccgtatctgaaatataaccatgcggatgcggaattt | |
| | caggaacgcctgaaagaagataccagctttggcggcaacctgggccgcgcggtgtttcag | |
| | gcgaaaaaacgcgtgctggaaccgctgggcctggtggaagaaccggtgaaaaccgcgccg | |
| | ggcaaaaaacgcccggtggaacatagcccggcggaaccggatagcagcagcggcaccggc | |
| | aaagcgggccagcagccggcgcgcaaacgcctgaactttggccagaccggcgatgcggat | |
| | agcgtgccggatccgcagccgctgggccagccgccggcggcgccgagcggcctgggcacc | |
| | aacaccatggcgaccggcagcggcgcgccgatggcggataacaacgaaggcgcggatggc | |
| | gtgggcaacagcagcggcaactggcattgcgatagcacctggatgggcgatcgcgtgatt | |
| | accaccagcacccgcacctgggcgctgccgacctataacaaccatctgtataaacagatt | |
| | agcagccagagcggcgcgagcaacgataaccattattttggctatagcaccccgtggggc | |
| | tattttgattttaaccgctttcattgccattttagcccgcgcgattggcagcgcctgatt | |
| | aacaacaactggggctttcgcccgaaacgcctgaactttaaactgtttaacattcaggtg | |
| | aaagaagtgacccagaacgatggcaccaccaccattgcgaacaacctgaccagcaccgtg | |
| | caggtgtttaccgatagcgaatatcagctgccgtatgtgctgggcagcgcgcatcagggc | |
| | tgcctgccgccgtttccggcggatgtgtttatggtgccgcagtatggctatctgaccctg | |
| | aacaacggcagccaggcggtgggccgcagcagcttttattgcctggaatattttccgagc | |
| | cagatgctgcgcaccggcaacaactttacctttagctatacctttgaagatgtgccgttt | |
| | catagcagctatgcgcatagccagagcctggatcgcctgatgaacccgctgattgatcag | |
| | tatctgtattatctgagcaaaaccaacgcgccgagcggcaccaccaccatgagccgcctg | |
| | cagtttagccaggcgggcgcgagcgatattcgcgatcagagccgcaactggctgccgggc | |
| | ccgtgctatcgccagcagcgcgtgagcaaaaccgcggcggataacaacaacagcgattat | |
| | agctggaccggcgcgaccaaatatcatctgaacggccgcgatagcctggtgaacccgggc | |
| | ccggcgatggcgagccataaagatgatgaagaaaaatattttccgcagagcggcgtgctg | |
| | atttttggcaaacaggatagcggcaaaaccaacgtggatattgaaaaagtgatgattacc | |
| | gatgaagaagaaattcgcaccaccaacccggtggcgaccgaacagtatggcagcgtgagc | |
| | accaacctgcagagcggcaacacccaggcggcgaccaccgatgtgaacacccagggcgtg | |
| | ctgccgggcatggtgtggcaggatcgcgatgtgtatctgcagggcccgatttgggcgaaa | |
| | attccgcataccgatggccattttcatccgagcccgctgatgggcggctttggcctgaaa | |
| | catccgccgccgcagattctgattaaaaacaccccggtgccggcgaacccgagcaccacc | |
| | tttagcgcggcgaaatttgcgagctttattacccagtatagcaccggccaggtgagcgtg | |
| | gaaattgaatgggaactgcagaaagaaaacagcaaacgctggaacccggaaattcagtat | |
| | accagcaactataacaaaagcgtgaacgtggattttaccgtggataccaacggcgtgtat | |
| | agcgaaccgcgcccgattggcacccgctatctgacccgcaacctg | |
| AAV2.V81 | atggcggcggatggctatctgccggattggctggaagataccctgagcgaaggcattcgc | 13 |
| | cagtggtggaaactgaaaccgggcccgccgccgctgaaaccggcggaacgccatcaggat | |
| | gatagccgcggcctggtgctgccgggctataaatatctgggcccgtttaacggcctggat | |
| | aaaggcgaaccggtgaacgaagcggatgcggcggcgctggaacatgataaagcgtatgat | |
| | cgccagctggatagcggcgataacccgtatctgaaatataaccatgcggatgcggaattt | |
| | caggaacgcctgaaagaagataccagctttggcggcaacctgggccgcgcggtgtttcag | |
| | gcgaaaaaacgcgtgctggaaccgctgggcctggtggaagaaccggtgaaaaccgcgccg | |
| | ggcaaaaaacgcccggtggaacatagcccggcggaaccggatagcagcagcggcaccggc | |
| | aaagcgggccagcagccggcgcgcaaacgcctgaactttggccagaccggcgatgcggat | |
| | agcgtgccggatccgcagccgctgggccagccgccggcggcgccgagcggcctgggcacc | |
| | aacaccatggcggcgggcagcggcgcgccgatggcggataacaacgaaggcgcggatggc | |
| | gtgggcaacagcagcggcaactggcattgcgatagcacctggatgggcgatcgcgtgatt | |
| | accaccagcacccgcacctgggcgctgccgacctataacaaccatctgtataaacagatt | |
| | agcagccagagcggcgcgagcaacgataaccattattttggctatagcaccccgtggggc | |
| | tattttgattttaaccgctttcattgccattttagcccgcgcgattggcagcgcctgatt | |
| | aacaacaactggggctttcgcccgaaacgcctgaactttaaactgtttaacattcaggtg | |
| | aaagaagtgacccagaacgatggcaccaccaccattgcgaacaacctgaccagcaccgtg | |
| | caggtgtttaccgatagcgaatatcagctgccgtatgtgctgggcagcgcgcatcagggc | |
| | tgcctgccgccgtttccggcggatgtgtttatggtgccgcagtatggctatctgaccctg | |
| | aacaacggcagccaggcggtgggccgcagcagcttttattgcctggaatattttccgagc | |
| | cagatgctgcgcaccggcaacaactttacctttagctatacctttgaagatgtgccgttt | |
| | catagcagctatgcgcatagccagagcctggatcgcctgatgaacccgctgattgatcag | |
| | tatctgtattatctgagcaaaaccaacgcgccgagcggcaccaccaccatgagccgcctg | |
| | cagtttagccaggcgggcgcgagcgatattcgcgatcagagccgcaactggctgccgggc | |
| | ccgtgctatcgccagcagcgcgtgagcaaaaccgcggcggataacaacaacagcgattat | |
| | agctggaccggcgcgaccaaatatcatctgaacggccgcgatagcctggtgaacccgggc | |
| | ccggcgatggcgagccataaagatgatgaagaaaaatattttccgcagagcggcgtgctg | |
| | atttttggcaaacaggatagcggcaaaaccaacgtggatattgaaaaagtgatgattacc | |
| | gatgaagaagaaattcgcaccaccaacccggtggcgaccgaacagtatggcagcgtgagc | |
| | accaacctgcagagcggcaacacccaggcggcgaccaccgatgtgaacacccagggcgtg | |
| | ctgccgggcatggtgtggcaggatcgcgatgtgtatctgcagggcccgatttgggcgaaa | |
| | attccgcataccgatggccattttcatccgagcccgctgatgggcggctttggcctgaaa | |
| | catccgccgccgcagattctgattaaaaacaccccggtgccggcgaacccgagcaccacc | |
| | tttagcgcggcgaaatttgcgagctttattacccagtatagcaccggccaggtgagcgtg | |
| | gaaattgaatgggaactgcagaaagaaaacagcaaacgctggaacccggaaattcagtat | |
| | accagcaactataacaaaagcgtgaacgtggattttaccgtggataccaacggcgtgtat | |
| | agcgaaccgcgcccgattggcacccgctatctgacccgcaacctg | |
| AAV2.V224 | atggcggcggatggctatctgccggattggctggaagataccctgagcgaaggcattcgc | 14 |
| | cagtggtggaaactgaaaccgggcccgccgccgccgaaaccggcggaacgccataaagat | |
| | gatagccgcggcctggtgctgccgggctataaatatctgggcccgtttaacggcctggat | |
| | aaaggcgaaccggtgaacgaagcggatgcggcggcgctggaacatgataaagcgtatgat | |
| | cgccagctggatagcggcgataacccgtatctgaaatataaccatgcggatgcggaattt | |
| | caggaacgcctgaaagaagataccagctttggcggcaacctggcgcgcgcggtgtttcag | |
| | gcgaaaaaacgcgtgctggaaccgctgggcctggtggaagaaccggtgaaaaccgcgccg | |
| | ggcaaaaaacgcccggtggaacatagcccggcggaaccggatagcagcagcggcaccggc | |
| | aaaagcggccagcagccggcgcgcaaacgcctgaactttggccagaccggcgatgcggat | |
| | agcgtgccggatccgcagccgctgggccagccgccggcggcgccgagcggcctgggcacc | |
| | aacaccatggcgagcggcagcggcgcgccgatggcggataacaacgaaggcgcggatggc | |
| | gtgggcaacagcagcggcaactggcattgcgatagcacctggatgggcgatcgcgtgatt | |
| | accaccagcacccgcacctgggcgctgccgacctataacaaccatctgtataaacagatt | |
| | agcagccagagcggcgcgagcaacgataaccattattttggctatagcaccccgtggggc | |
| | tattttgattttaaccgctttcattgccattttagcccgcgcgattggcagcgcctgatt | |
| | aacaacaactggggctttcgcccgaaacgcctgaactttaaactgtttaacattcaggtg | |
| | aaagaagtgacccagaacgatggcaccaccaccattgcgaacaacctgaccagcaccgtg | |
| | caggtgtttaccgatagcgaatatcagctgccgtatgtgctgggcagcgcgcatcagggc | |
| | tgcctgccgccgtttccggcggatgtgtttatggtgccgcagtatggctatctgaccctg | |
| | aacaacggcagccaggcggtgggccgcagcagcttttattgcctggaatattttccgagc | |
| | cagatgctgcgcaccggcaacaactttacctttagctatacctttgaagatgtgccgttt | |
| | catagcagctatgcgcatagccagagcctggatcgcctgatgaacccgctgattgatcag | |
| | tatctgtattatctgagccgcaccaacaccccgagcggcaccaccacccagagccgcctg | |
| | cgctttagccaggcgggcgcgagcgatattcgcgatcagagccgcaactggctgccgggc | |
| | ccgtgctatcgccagcagcgcgtgagcaaaaccgcggcggataacaacaacagcgattat | |
| | agctggaccggcgcgaccaaatatcatctgaacggccgcgatagcctggtgaacccgggc | |
| | accgcgatggcgagccataaagatgatgaagaaaaatattttccgcagagcggcgtgctg | |
| | atttttggcaaacaggatagcgaaaaaaccaacgtggatattgaacgcgtgatgattacc | |
| | gatgaagaagaaattcgcaccaccaacccggtggcgaccgaacagtatggcagcgtgagc | |
| | accaacctgcagagcggcaacacccaggcggcgaccagcgatgtgaacacccagggcgtg | |
| | ctgccgggcatggtgtggcaggatcgcgatgtgtatctgcagggcccgatttgggcgaaa | |
| | attccgcataccgatggccattttcatccgagcccgctgatgggcggctttggcctgaaa | |
| | catccgccgccgcagattctgattaaaaacaccccggtgccggcgaacccgagcaccacc | |
| | tttagcgcggcgaaatttgcgagctttattacccagtatagcaccggccaggtgagcgtg | |
| | gaaattgaatgggaactgcagaaagaaaacagcaaacgctggaacccggaaattcagtat | |
| | accagcaactataacaaaagcgtgaacgtggattttaccgtggataccaacggcgtgtat | |
| | agcgaaccgcgcccgattggcacccgctatctgacccgcaacctg | |
| AAV2.v326 | atggcggcggatggctatctgccggattggctggaagataccctgagcgaaggcattcgc | 15 |
| | cagtggtggaaactgaaaccgggcccgccgccgccgaaaccggcggaacgccataaagat | |
| | gatagccgcggcctggtgctgccgggctataaatatctgggcccgtttaacggcctggat | |
| | aaaggcgaaccggtgaacgaagcggatgcggcggcgctggaacatgataaagcgtatgat | |
| | cgccagctggatagcggcgataacccgtatctgaaatataaccatgcggatgcggaattt | |
| | caggaacgcctgaaagaagataccagctttggcggcaacctgggccgcgcggtgtttcag | |
| | gcgaaaaaacgcgtgctggaaccgctgggcctggtggaagaaccggtgaaaaccgcgccg | |
| | ggcaaaaaacgcccggtggaacatagcccggtggaaccggatagcagcagcggcaccggc | |
| | aaagcgggccagcagccggcgcgcaaacgcctgaactttggccagaccggcgatgcggat | |
| | agcgtgccggatccgcagccgctgggccagccgccggcggcgccgagcggcctgggcacc | |
| | aacaccatggcgaccggcagcggcgcgccgatggcggataacaacgaaggcgcggatggc | |
| | gtgggcaacagcagcggcaactggcattgcgatagcacctggatgggcgatcgcgtgatt | |
| | accaccagcacccgcacctgggcgctgccgacctataacaaccatctgtataaacagatt | |
| | agcagccagagcggcgcgagcaacgataaccattattttggctatagcaccccgtggggc | |
| | tattttgattttaaccgctttcattgccattttagcccgcgcgattggcagcgcctgatt | |
| | aacaacaactggggctttcgcccgaaacgcctgagctttaaactgtttaacattcaggtg | |
| | aaagaagtgacccagaacgatggcaccaccaccattgcgaacaacctgaccagcaccgtg | |
| | caggtgtttaccgatagcgaatatcagctgccgtatgtgctgggcagcgcgcatcagggc | |
| | tgcctgccgccgtttccggcggatgtgtttatggtgccgcagtatggctatctgaccctg | |
| | aacaacggcagccaggcggtgggccgcagcagcttttattgcctggaatattttccgagc | |
| | cagatgctgcgcaccggcaacaactttacctttagctatacctttgaagatgtgccgttt | |
| | catagcagctatgcgcatagccagagcctggatcgcctgatgaacccgctgattgatcag | |
| | tatctgtattatctgagcaaaaccaacgcgccgagcggcaccaccaccatgagccgcctg | |
| | cagtttagccaggcgggcgcgagcgatattcgcgatcagagccgcaactggctgccgggc | |
| | ccgtgctatcgccagcagcgcgtgagcaaaaccgcggcggataacaacaacagcgattat | |
| | agctggaccggcgcgaccaaatatcatctgaacggccgcgatagcctggtgaacccgggc | |
| | ccggcgatggcgaaccataaagatgatgaagaaaaatattttccgcagagcggcgtgctg | |
| | atttttggcaaacagggcagcaacaaaaccaacgtggatattgaaaaagtgatgattacc | |
| | gatgaagaagaaattcgcaccaccaacccggtggcgaccgaacagtatggcagcgtgagc | |
| | accaacctgcagagcggcaacacccaggcggcgaccagcgatgtgaacacccagggcgtg | |
| | ctgccgggcatggtgtggcaggatcgcgatgtgtatctgcagggcccgatttgggcgaaa | |
| | attccgcataccgatggccattttcatccgagcccgctgatgggcggctttggcctgaaa | |
| | catccgccgccgcagattctgattaaaaacaccccggtgccggcgaacccgagcaccacc | |
| | tttagcgcggcgaaatttgcgagctttattacccagtatagcaccggccaggtgagcgtg | |
| | gaaattgaatgggaactgcagaaagaaaacagcaaacgctggaacccggaaattcagtat | |
| | accagcaactataacaaaagcgtgaacgtggattttaccgtggataccaacggcgtgtat | |
| | agcgaaccgcgcccgattggcacccgctatctgacccgcaacctg | |
| AAV2.V358 | atggcggcggatggctatctgccggattggctggaagataccctgagcgaaggcattcgc | 16 |
| | cagtggtggaaactgaaaccgggcccgccgccgccgaaaccggcggaacgccataaagat | |
| | gatagccgcggcctggtgctgccgggctataaatatctgggcccgtttaacggcctggat | |
| | aaaggcgaaccggtgaacgaagcggatgcggcggcgctggaacatgataaagcgtatgat | |
| | cgccagctggatagcggcgataacccgtatctgaaatataaccatgcggatgcggaattt | |
| | caggaacgcctgaaagaagataccagctttggcggcaacctgggccgcgcggtgtttcag | |
| | gcgaaaaaacgcgtgctggaaccgctgggcctggtggaagaaccggtgaaaaccgcgccg | |
| | ggcaaaaaacgcccggtggaacatagcccggtggaaagcgatagcagcagcggcaccggc | |
| | aaagcgggccagcagccggcgcgcaaacgcctgaactttggccagaccggcgatgcggat | |
| | agcgtgccggatccgcagccgctgggccagccgccggcggcgccgagcggcctgggcacc | |
| | aacaccatggcgaccggcagcggcgcgccgatggcggataacaacgaaggcgcggatggc | |
| | gtgggcaacagcagcggcaactggcattgcgatagcacctggatgggcgatcgcgtgatt | |
| | accaccagcacccgcacctgggcgctgccgacctataacaaccatctgtataaacagatt | |
| | agcagccagagcggcgcgagcaacgataaccattattttggctatagcaccccgtggggc | |
| | tattttgattttaaccgctttcattgccattttagcccgcgcgattggcagcgcctgatt | |
| | aacaacaactggggctttcgcccgaaacgcctgagctttaaactgtttaacattcaggtg | |
| | aaagaagtgacccagaacgatggcaccaccaccattgcgaacaacctgaccagcaccgtg | |
| | caggtgtttaccgatagcgaatatcagctgccgtatgtgctgggcagcgcgcatcagggc | |
| | tgcctgccgccgtttccggcggatgtgtttatggtgccgcagtatggctatctgaccctg | |
| | aacaacggcagccaggcggtgggccgcagcagcttttattgcctggaatattttccgagc | |
| | cagatgctgcgcaccggcaacaactttacctttagctatacctttgaagatgtgccgttt | |
| | catagcagctatgcgcatagccagagcctggatcgcctgatgaacccgctgattgatcag | |
| | tatctgtattatctgagcaaaaccaacgcgccgagcggcaccaccaccatgagccgcctg | |
| | cagtttagccaggcgggcgcgagcgatattcgcgatcagagccgcaactggctgccgggc | |
| | ccgtgctatcgccagcagcgcgtgagcaaaaccgcggcggataacaacaacagcgattat | |
| | agctggaccggcgcgaccaaatatcatctgaacggccgcgatagcctggtgaacccgggc | |
| | ccggcgatggcgagccataaagatgatgaagaaaaatattttccgcagagcggcgtgctg | |
| | atttttggcaaacagggcagcaacaaaaccaacgtggatattgaaaaagtgatgattacc | |
| | gatgaagaagaaattcgcaccaccaacccggtggcgaccgaacagtatggcagcgtgagc | |
| | accaacctgcagagcggcaacacccaggcggcgaccagcgatgtgaacacccagggcgtg | |
| | ctgccgggcatggtgtggcaggatcgcgatgtgtatctgcagggcccgatttgggcgaaa | |
| | attccgcataccgatggccattttcatccgagcccgctgatgggcggctttggcctgaaa | |
| | catccgccgccgcagattctgattaaaaacaccccggtgccggcgaacccgagcaccacc | |
| | tttagcgcggcgaaatttgcgagctttattacccagtatagcaccggccaggtgagcgtg | |
| | gaaattgaatgggaactgcagaaagaaaacagcaaacgctggaacccggaaattcagtat | |
| | accagcaactataacaaaagcgtgaacgtggattttaccgtggataccaacggcgtgtat | |
| | agcgaaccgcgcccgattggcacccgctatctgacccgcaacctg | |

### EXAMPLES

### Example 1. Capsid Proteins for Targeting Deep Brain Structures

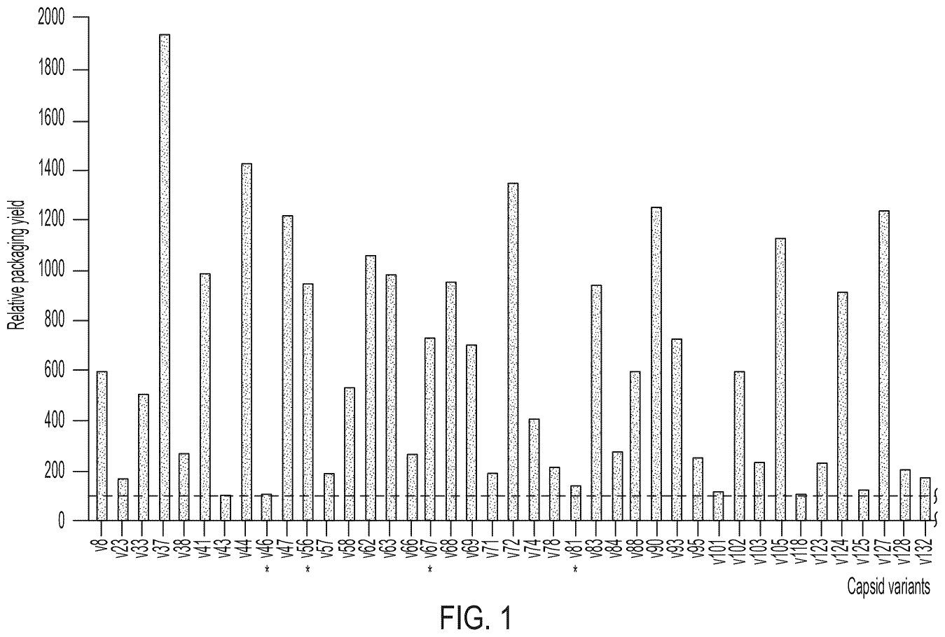

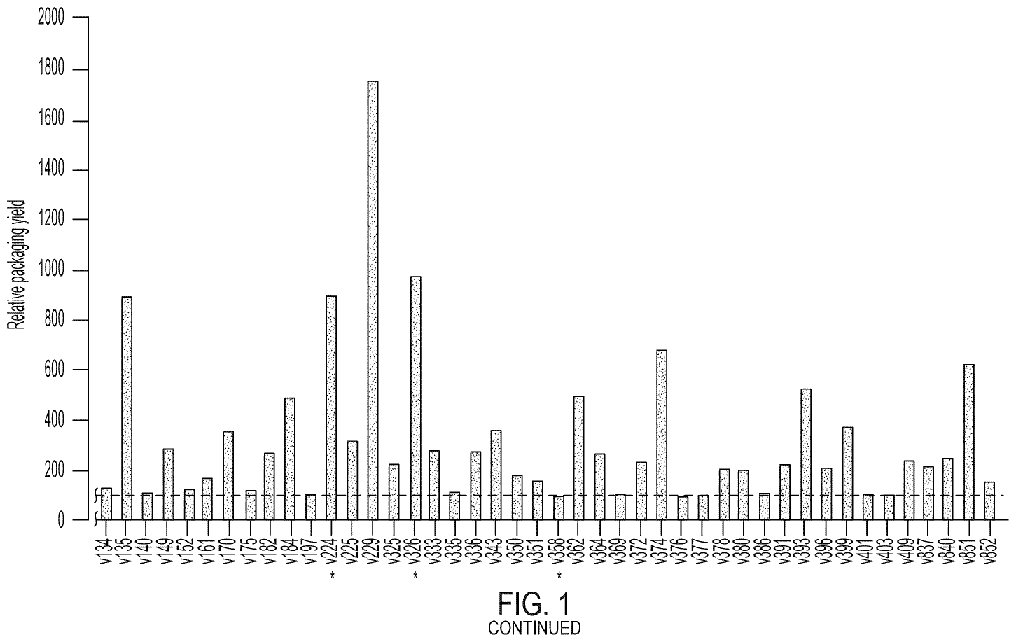

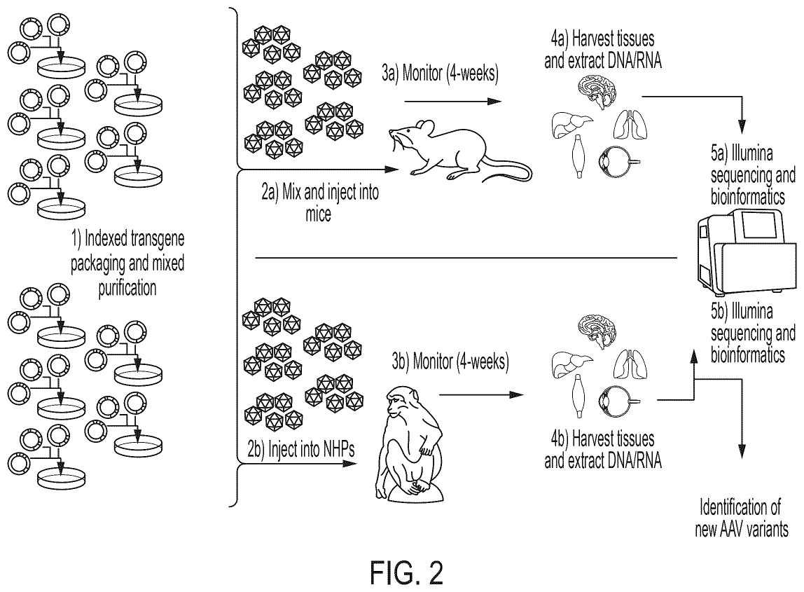

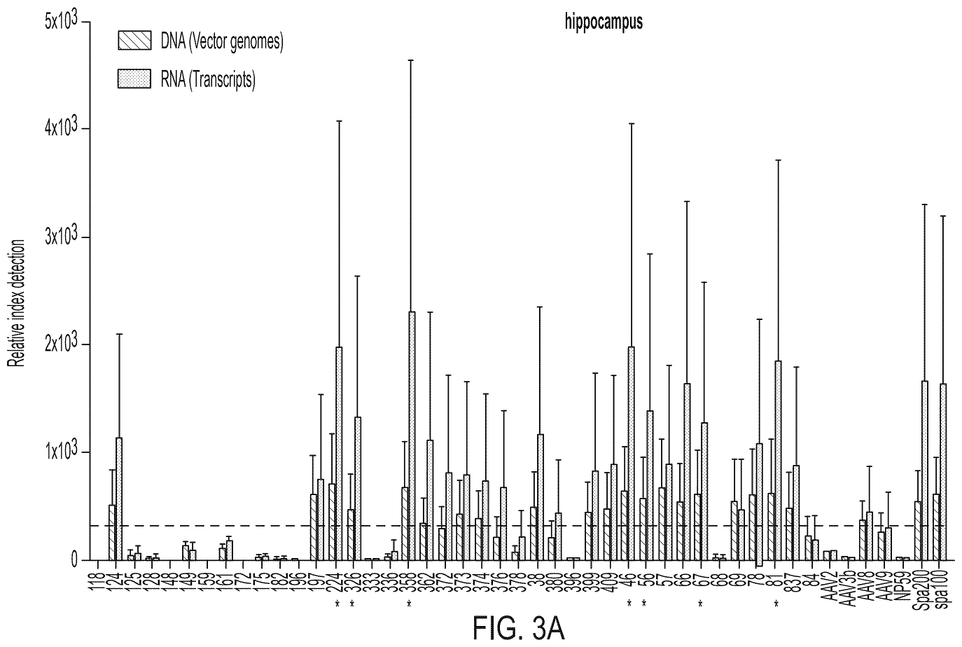

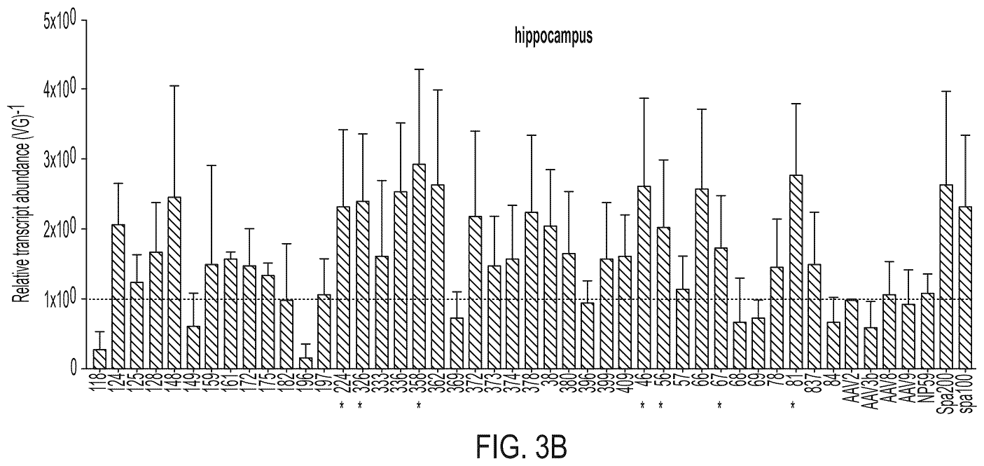

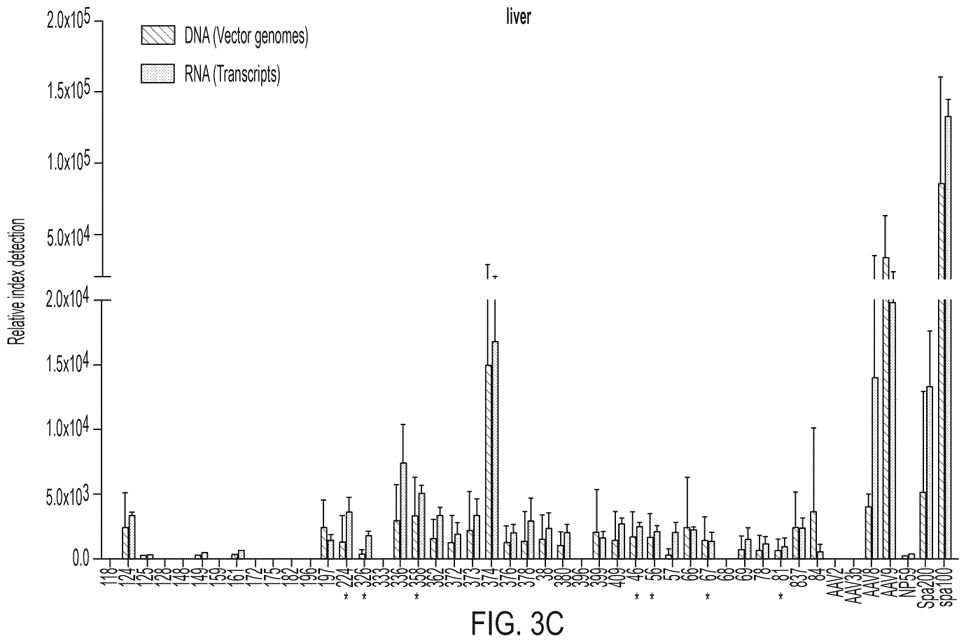

A set of AAV2 capsid protein variants were identified from human tissue samples, 86 of which showed packaging yields that were equal to, or better than wild-type AAV2 capsid (FIGS. 1A-1B) These capsid proteins were screened in mice and non-human primates (NHPs) using a barcoded non-coding RNA transcript (barcoded Tough Decoy; bcTuD) (Xu et al. Hum Gene Ther. 2019 Aug. 1; 30(8):946-956) to identify capsids that can transduce central nervous system (CNS) tissues (FIG. 2). Seven AAV2 capsid protein variants (AAV2.V46, AAV2.V56, AAV2.V67, AAV2.V81, AAV2.V224, Aav2.v326 and AAV2.V358) were identified as having specific ability for targeting cells of the central nervous system and for having the ability to cross the blood-brain barrier (FIG. 3, see variants with asterisks). AAV2.V46 (SEQ ID NO: 2), AAV2.V56 (SEQ ID NO: 3), AAV2.V67 (SEQ ID NO: 4), AAV2.V81 (SEQ ID NO: 5), AAV2.V224 (SEQ ID NO: 6), Aav2.v326 (SEQ ID NO: 7), and AAV2.V358 (SEQ ID NO: 8) comprised amino acid substitutions relative to wild-type AAV2 capsid protein (SEQ ID NO: 1), the specific substitutions of which are shown in Table 4. Mice were then injected via intrahippocampal injections with select AAV2 capsid variants, as well as wild-type AAV2, AAV3b, AAV8, AAV9, NP59, Spark 100, and Spark200. As AAV9 is considered the current gold standard for CNS transduction, this capsid was used as a control for brain penetrance. Spark 100 and Spark200 are proprietary capsids currently used in clinical trials (e.g., as described by Elkouby et al. Mol Ther Methods Clin Dev. 2021 Nov. 24; 24:20-29), whereas AAV3b and AAV8 are of serotypes not known to penetrate the blood-brain barrier (BBB). AAV2.V46, AAV2.V56, AAV2.V67, AAV2.V81, AAV2.V224, Aav2.v326 and AAV2.V358 demonstrated strong transduction following intrahippocampal injections in adult mice (FIGS. 3A-3B). These variants showed levels of transduction 4- to 5-fold greater than those achieved by AAV9, and equal levels of transduction as achieved by Spark100 and Spark200. Additionally, these AAV2 capsid variants showed lower levels of transduction in the liver compared to AAV8, AAV9, Spark100, and Spark200 (FIG. 3C), indicating that the leakage of vectors packaged with the AAV2 variants disclosed herein have lower risk of off-target expression than other serotypes known in the art.

TABLE 4
Variant mutations relative to wild-type AAV2

| VP | VR | v46 | v56 | v67 | v81 | v224 | v326 | v358 |
|---|---|---|---|---|---|---|---|---|
| VP1u | | K39Q | K39Q | K39Q | P32L | G115A | | |
| | | N66S | | A70V | K36Q | | | |
| | | A70V | | | | | | |
| VP2 | | V151A | S149F | V151A | V151A | V151A | | P153S |
| | | | V151A | | | A162S | | |
| VP3 | | | | | T205A | T205S | | |
| | I | | | | | | | |
| | | | | | | | N312S | N312S |
| | II | | | | | | | |
| | III | | | | | | | |
| | IV | R447K | R447K | R447K | R447K | Q461R | R447K | R447K |
| | | T450A | T450A | T450A | T450A | | T450A | T450A |
| | | Q457M | Q457M | Q457M | Q457M | | Q457M | Q457M |
| | V | S492A | S492A | S492A | S492A | S492A | S492A | S492A |
| | | E499D | E499D | E499D | E499D | E499D | E499D | E499D |
| | VI | F533Y | F533Y | F533Y | F533Y | P521T | S525N | F533Y |
| | | | | | | F533Y | F533Y | |
| | VII | G546D | G546D | G546D | G546D | G546D | E548N | E548N |
| | | E548G | E548G | E548G | E548G | K556R | | |
| | VIII | R585S | R585S | R585S | R585S | R585S | R585S | R585S |
| | | R588T | R588T | R588T | R588T | R588T | R588T | R588T |
| | | A593T | A593T | A593T | A593T | A593S | A593S | A593S |

### Example 2. AAV2 Capsid Variants Cross the Blood-Brain Barrier and Penetrate Deep Brain Tissues

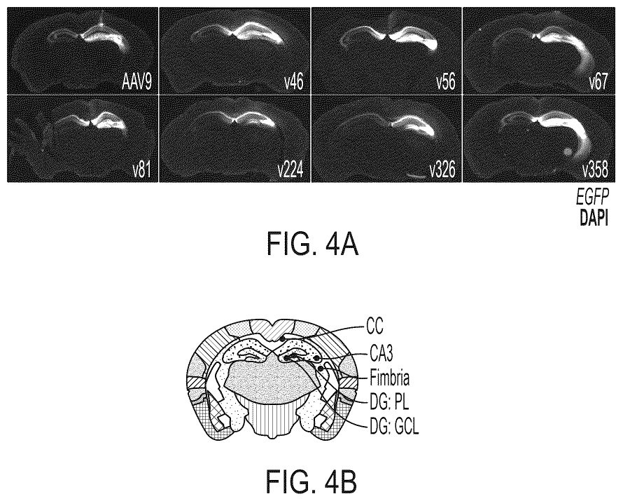

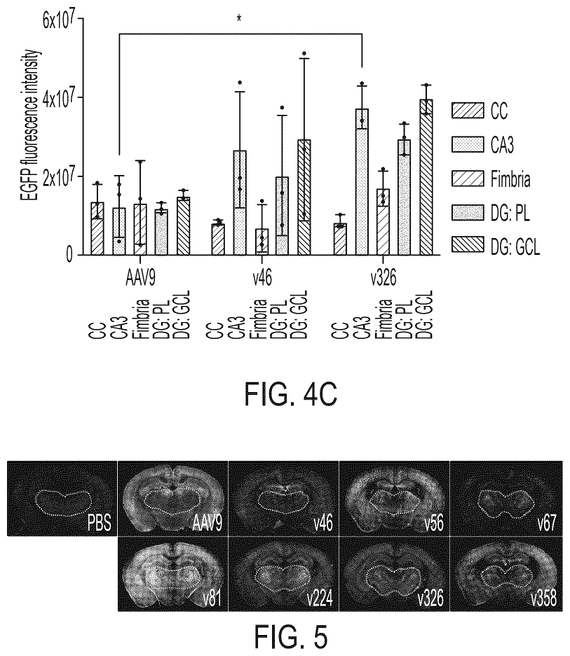

AAV2.V46, AAV2.V56, AAV2.V67, AAV2.V81, AAV2.V224, Aav2.v326 and AAV2.V358 were packaged with self-complementary (sc) AAV vectors encoding EGFP under the control of a chicken beta-actin promoter (scAAV-CB6-Egfp). The resulting rAAVs were injected into adult mice via intrahippocampal injections. It was observed that the AAV2 variants disclosed herein transduced the hippocampus as well as AAV9 (FIG. 4A). Surprisingly, two of the tested variants (AAV2.V46 and Aav2.v326) also conferred differential transgene expression within separate structures of the hippocampus (FIGS. 4B-4C).

The ability of the AAV2 capsid variants to cross the blood-brain barrier (BBB) was tested following intravenous injections into neonatal mice. scAAV-CB6-Egfp vector packaged with AAV2.V46, AAV2.V56, AAV2.V67, AAV2.V81, AAV2.V224, Aav2.v326 and AAV2.V358 capsids were injected into facial veins of P1 mice, and the brains cryosectioned and stained for EGFP expression. FIG. 5 demonstrates that all seven of the AAV2 capsid variants disclosed herein were able to cross the blood-brain barrier and confer expression of the transgene in deep brain structures of mice. Of all the capsid variants, AAV v56 performed similarly to AAV9, whereas AAV2.V81 led to the highest level of EGFP expression in the brain, even outperforming AAV9. In fact, AAV9 did not appear to transduce the thalamus as well as other brain structures (FIG. 5), indicating that AAV9 does not efficiently penetrate deep brain structures. Conversely, AAV2. V81 transduces the thalamus more efficiently than AAV9, and AAV2.V67, AAV2.V224, Aav2.v326, and AAV2.V358 all show clear transduction in the thalamus as well.

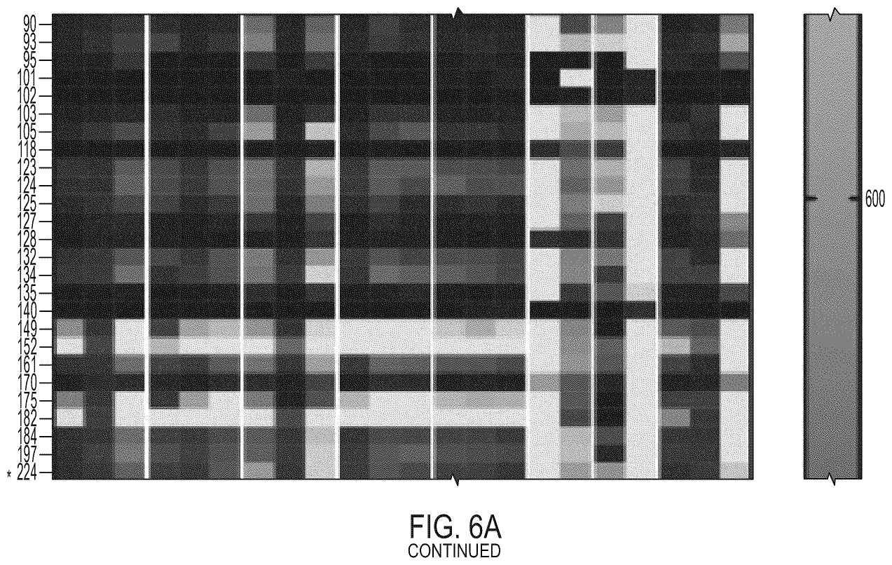

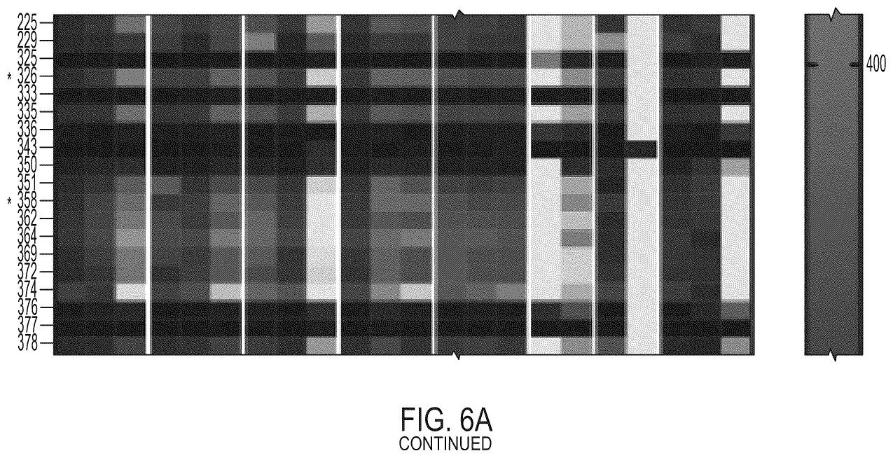

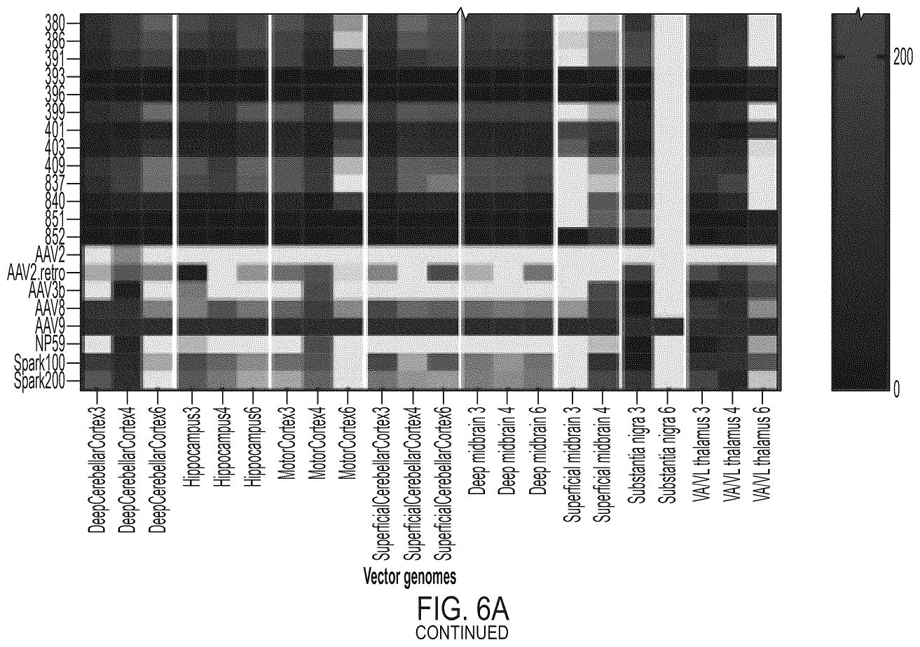

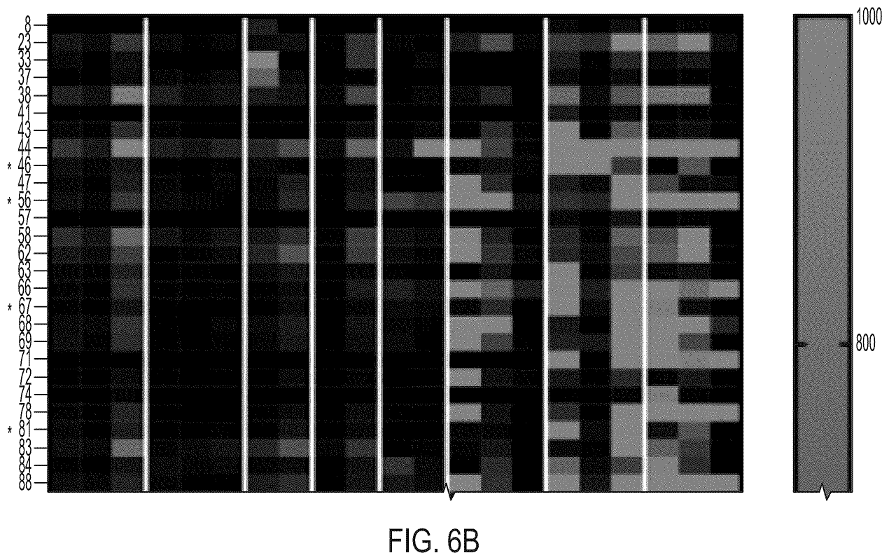

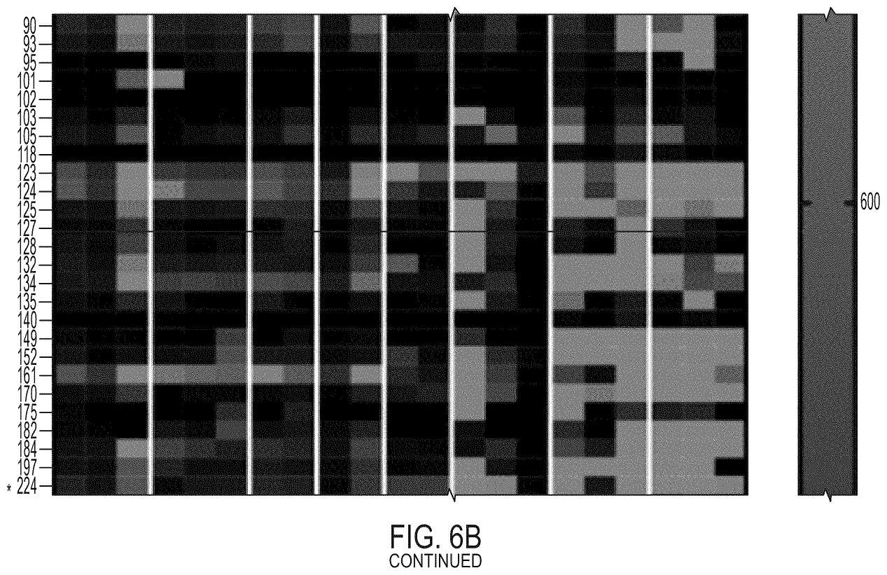

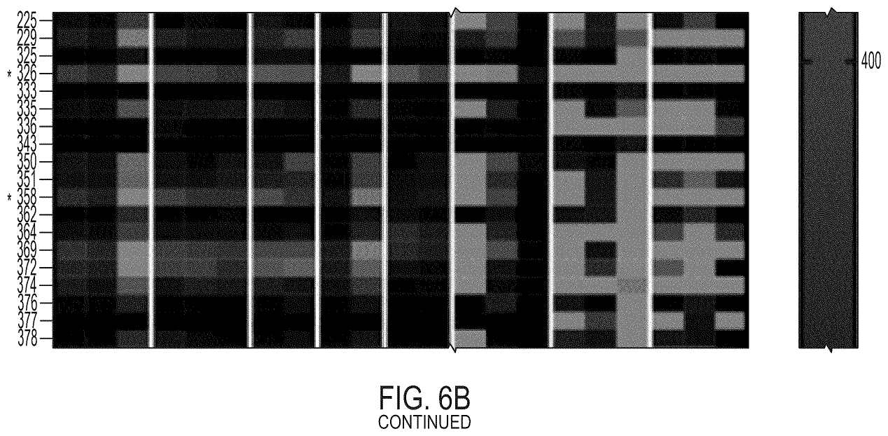

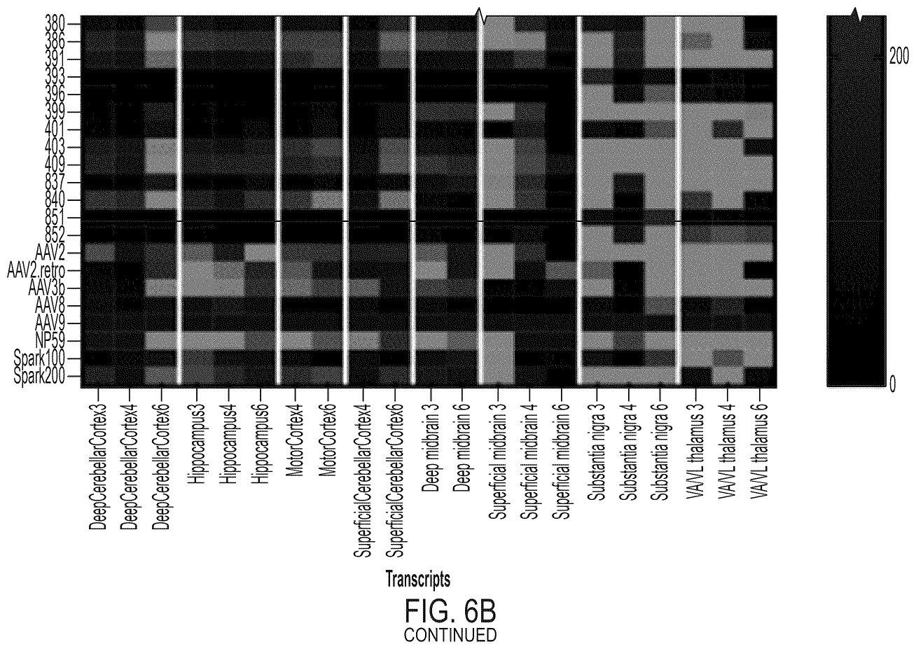

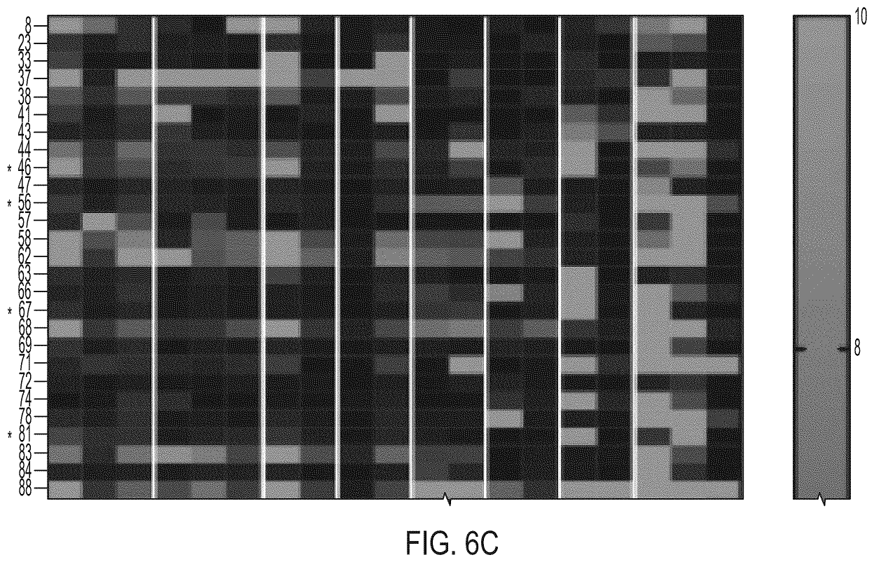

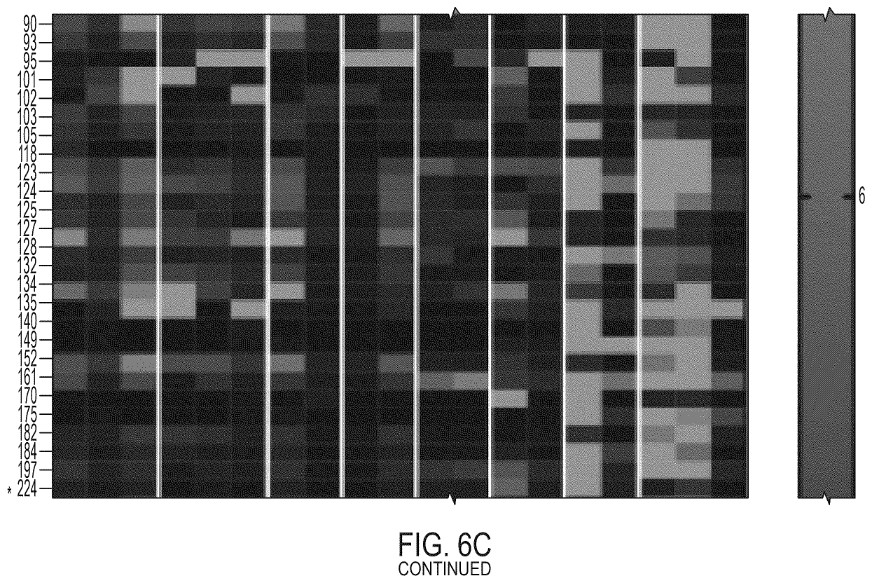

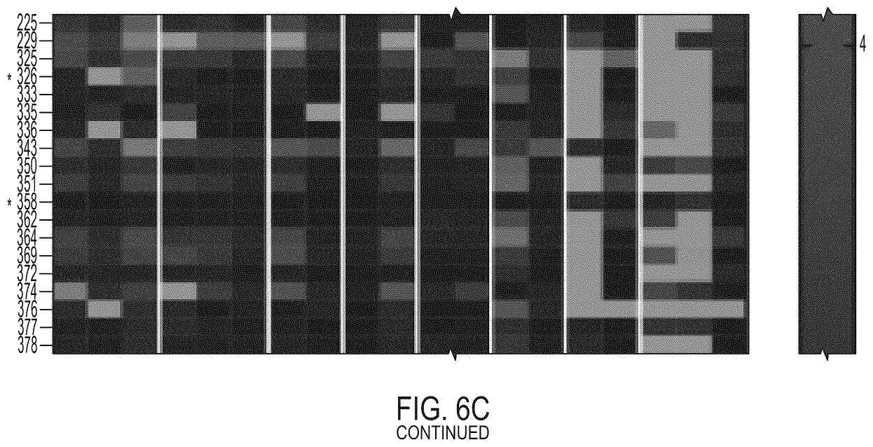

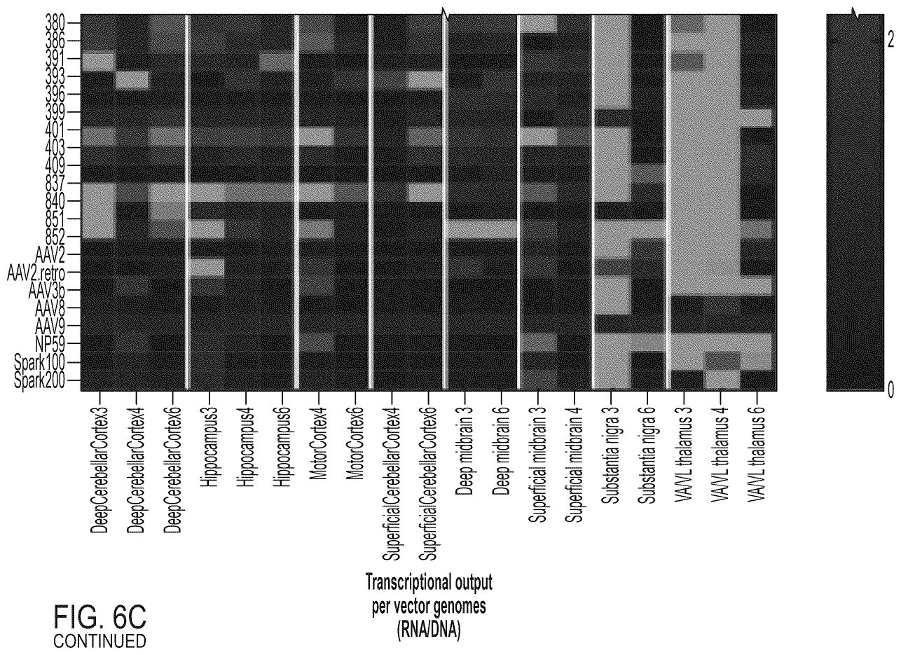

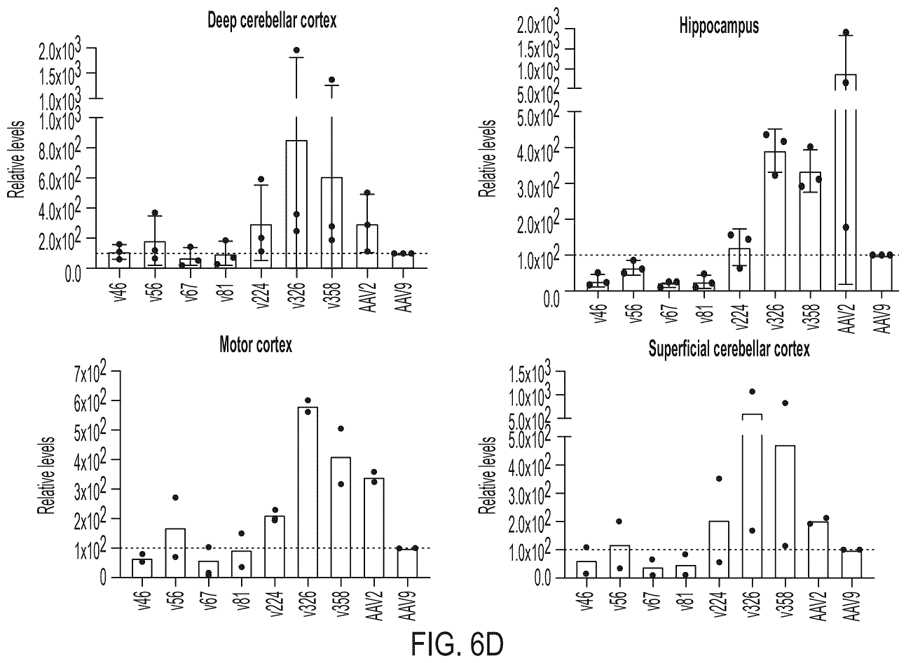

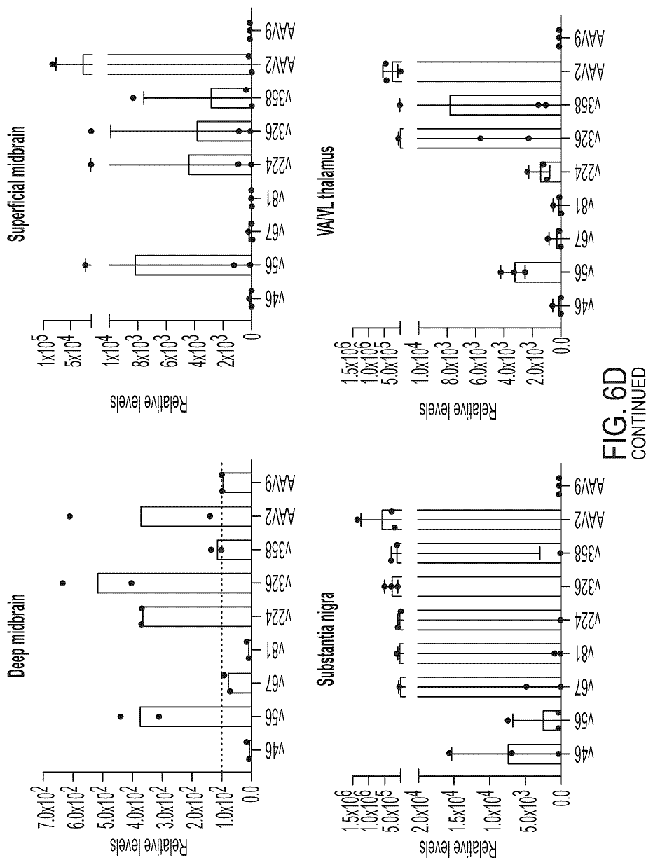

The seven AAV2 capsid variants described herein were likewise tested for their efficacy and tropism in non-human primates (NHPs). The 86 AAV2 capsid protein variants identified in Example 1 (FIGS. 1A-1B) were analyzed for their ability to transduce specific structures of the brain compared to AAV9 (FIGS. 6A-6N). Quantification of the relative numbers of vector genomes, mRNA transcripts, and transcriptional output per vector genomes showed that AAV2.V46, AAV2.V56, AAV2.V67, AAV2.V81, AAV2.V224, Aav2.v326 and AAV2.V358 capsids conferred greater expression in deep brain tissues, such as the thalamus, compared to AAV9. These findings were further confirmed by injecting NHPs through intracerebroventricular (ICV) injections with AAV2 capsid variants, wild-type AAV2, and AAV9 (FIGS. 6M-6N). Quantification of the relative levels of vector genomes showed that AAV2.V224, Aav2.v326, and AAV2.V358 performed better than AAV9 in penetrating several brain structures, including deep brain tissues. Notably, AAV9 is nearly undetected in the NHP thalamus, whereas AAV2.V56, Aav2.v326, and AAV2.V358 all showed relatively high expression in the thalamus.

### Additional Embodiments

Additional embodiments of the present disclosure are encompassed by the following numbered paragraphs:

1. A method for delivering a transgene to central nervous system (CNS) cell of a subject, the method comprising administering to the subject a recombinant adeno-associated virus (rAAV) comprising:
(i) an isolated nucleic acid comprising a transgene encoding one or more gene products flanked by adeno-associated virus (AAV) inverted terminal repeats (ITRs); and
(ii) an adeno-associated virus (AAV) capsid protein, wherein the capsid protein comprises one or more amino acid substitutions corresponding to position P32, K39, N66, A70, G115, S149, V151, P153, A126, T205, N312, R447, T450, Q457, Q461, S492, E499, P521, S525, F533, G546, E548, K556, R585, R588, and/or A593 with reference to amino acid position numbering of a wild-type AAV2 capsid protein, optionally wherein the wild-type AAV2 capsid protein comprises the amino acid sequence set forth in SEQ ID NO: 1.

2. The method of paragraph 1, wherein the capsid protein comprises an amino acid substitution corresponding to P32L, K39Q, N66S, A70V, G115A, S149F, V151A, P153S, A162S, T205A, T205S, N312S, R447K, T450A, Q457M, Q461R, S492A, E499D, P521T, S525N, F533Y, G546D, E548G, E548N, K556R, R585S, R588T, A593T, and/or A593S with reference to amino acid position numbering of a wild-type AAV2 capsid protein, optionally wherein the wild-type AAV2 capsid protein comprises the amino acid sequence set forth in SEQ ID NO: 1.

3. The method of paragraph 1 or 2, wherein the capsid protein comprises amino acid substitutions at amino acid positions corresponding to positions K39, N66, A70, V151, R447, T450, Q457, S492, E499, F533, G546, E548, R585, R588, and A593 with reference to amino acid position numbering of a wild-type AAV2 capsid protein.

4. The method of any one of paragraphs 1 to 3, wherein the capsid protein comprises amino acid substitutions corresponding to K39Q, N66S, A70V, V151A, R447K, T450A, Q457M, S492A, E499D, F533Y, G546D, E548G, R585S, R588T, and A593T with reference to amino acid position numbering of a wild-type AAV2 capsid protein.

5. The method of any one of paragraphs 1 to 4, wherein the capsid protein comprises the amino acid sequence set forth in SEQ ID NO: 2.

6. The method of paragraph 1 or 2, wherein the capsid protein comprises amino acid substitutions corresponding to positions K39, S149, V151, R447, T450, Q457, S492, E499, F533, G546, E548, R585, R588, and A593 with reference to amino acid position numbering of a wild-type AAV2 capsid protein.

7. The method of paragraph 6, wherein the capsid protein comprises amino acid substitutions corresponding to K39Q, S149F, V151A, R447K, T450A, Q457M, S492A, E499D, F533Y, G546D, E548G, R585S, R588T, and A593T with reference to amino acid position numbering of a wild-type AAV2 capsid protein.

8. The method of paragraph 6 or 7, wherein the capsid protein comprises the amino acid sequence set forth in SEQ ID NO: 3.

9. The method of paragraph 1 or 2, wherein the capsid protein comprises amino acid substitutions corresponding to positions K39, A70, V151, R447, T450, Q457, S492, E499, F533, G546, E548, R585, R588, and A593 with reference to amino acid position numbering of a wild-type AAV2 capsid protein.

10. The method of paragraph 8 or 9, wherein the capsid protein comprises amino acid substitutions corresponding to K39Q, A70V, V151A, R447K, T450A, Q457M, S492A, E499D, F533Y, G546D, E548G, R585S, R588T, and A593T with reference to amino acid position numbering of a wild-type AAV2 capsid protein.

11. The method of any one of paragraphs 8 to 10, wherein the capsid protein comprises the amino acid sequence set forth in SEQ ID NO: 4.

12. The method of paragraph 1 or 2, wherein the capsid protein comprises amino acid substitutions corresponding to positions P32, K39, V151, T205, R447, T450, Q457, S492, E499, F533, G546, E548, R585, R588, and A593 with reference to amino acid position numbering of a wild-type AAV2 capsid protein.

13. The method of paragraph 12, wherein the capsid protein comprises amino acid substitutions corresponding to P32L, K39Q, V151A, T205A, R447K, T450A, Q457M, S492A, E499D, F533Y, G546D, E548G, R585S, R588T, and A593T with reference to amino acid position numbering of a wild-type AAV2 capsid protein.

14. The method of paragraph 12 or 13, wherein the capsid protein comprises the amino acid sequence set forth in SEQ ID NO: 5.

15. The method of paragraph 1 or 2, wherein the capsid protein comprises amino acid substitutions corresponding to positions G115, V151, A162, T205, Q461, S492, E499, P521, F533, G546, K556, R585, R588, and A593 with reference to amino acid position numbering of a wild-type AAV2 capsid protein.

16. The method of paragraph 15, wherein the capsid protein comprises amino acid substitutions corresponding to G115A, V151A, A162S, T205S, Q461R, S492A, E499D, P521T, F533Y, G546D, K566R, R585S, R588T, and A593S with reference to amino acid position numbering of a wild-type AAV2 capsid protein.

17. The method of paragraph 15 or 16, wherein the capsid protein comprises the amino acid sequence set forth in SEQ ID NO: 6.

18. The method of paragraph 1 or 2, wherein the capsid protein comprises amino acid substitutions corresponding to positions N312, R447, T450, Q457, S492, E499, S525, F533, E548, R585, R588, and A593 with reference to amino acid position numbering of a wild-type AAV2 capsid protein.

19. The method of paragraph 18, wherein the capsid protein comprises amino acid substitutions corresponding to N312S, R447K, T450A, Q457M, S492A, E499D, S525N, F533Y, E548N, R585S, R588T, and A593S with reference to amino acid position numbering of a wild-type AAV2 capsid protein.

20. The method of paragraph 18 or 19, wherein the capsid protein comprises the amino acid sequence set forth in SEQ ID NO: 7.

21. The method of paragraph 1 or 2, wherein the capsid protein comprises amino acid substitutions corresponding to positions P153, N312, R447, T450, Q457, S492, E499, F533, E548, R585, R588, and A593 with reference to amino acid position numbering of a wild-type AAV2 capsid protein.

22. The method of paragraph 21, wherein the capsid protein comprises amino acid substitutions corresponding to P153S, N312S, R447K, T450A, Q457M, S492A, E499D, F533Y, E548N, R585S, R588T, and A593S with reference to amino acid position numbering of a wild-type AAV2 capsid protein.

23. The method of paragraph 21 or 22, wherein the capsid protein comprises the amino acid sequence set forth in SEQ ID NO: 8.

24. The method of any one of the preceding paragraphs, wherein the capsid protein has at least 90%, 95%, 97%, or 98% sequence identity to the amino acid sequence set forth in SEQ ID NO: 1.

25. A method for delivering a transgene to a cell of the central nervous system (CNS) in a subject, the method comprising administering to the subject a recombinant adeno-associated virus (rAAV) comprising:
(i) an isolated nucleic acid comprising a transgene encoding one or more gene products flanked by adeno associated virus (AAV) inverted terminal repeats (ITRs); and
(ii) an AAV capsid protein comprising the amino acid sequence set forth in any one of SEQ ID NOs: 2-8.

26. The method of any one of the preceding paragraphs, wherein the administration comprises intracranial injection, optionally intrahippocampal injection, intraparenchymal injection, intravenous (IV) injection, or intracerebroventricular (ICV) injection.

27 The method of any one of the preceding paragraphs, wherein the cell of the CNS is present in the corpus callosum, cornu ammonis, fimbria, polymorph layer of the dentate gyrus, and granule cell layer of the dentate gyrus of the hippocampus of the subject.

28. The method of paragraph 27, wherein the cell of the CNS is a neuron, astrocyte, oligodendrocyte, Muller glial cell, Schwann cell, enteric glial cell, or microglial cell.

29. The method of any one of the preceding paragraphs, wherein the subject is a mammal, optionally wherein the mammal is a human.

30. The method of any one of the preceding paragraphs, wherein the nucleic acid sequence encoding the one or more gene products is operably linked to a promoter.

31. The method of any one of the preceding paragraphs, wherein the promoter is a CNS-specific promoter, further optionally wherein the CNS-specific promoter is a glial fibrillary acidic protein (GFAP) promoter, gfaABC1D promoter, gfa28/gfaABD promoter, ALDH1L1 promoter, gfa2 promoter, gfa2(B₃) promoter, Mbp promoter, MAG promoter, Cbh promoter, F4/80 promoter, CD68 promoter, CD11B promoter, RLBP1 promoter, ProB2 promoter, Mpz promoter, or Cnp promoter.

32. The method of any one of the preceding paragraphs, wherein the one or more gene products comprise a protein or an inhibitory nucleic acid.

33. The method of any one of the preceding paragraphs, wherein the rAAV crosses the blood-brain barrier of the subject after the administration.

34. A recombinant expression vector comprising a nucleic acid encoding a polypeptide comprising the sequence as set forth in any one of SEQ ID NOs: 2-8.

35. A recombinant expression vector comprising a nucleic acid comprising the sequence as set forth in any one of SEQ ID NOs: 10-16.

36. An isolated AAV capsid protein comprising an amino acid sequence comprising the sequence as set forth in any one of SEQ ID NOs: 2-8.

37. A recombinant AAV (rAAV) comprising the isolated AAV capsid of paragraph 36.

38. A composition comprising the rAAV of paragraph 37 and a pharmaceutically acceptable carrier.

39. A host cell comprising the recombinant expression vector of paragraph 34 or 35, the isolated AAV capsid protein of paragraph 36, or the rAAV of paragraph 38.

40. A method for delivering a transgene to a subject comprising administering the rAAV of paragraph 37 to a subject, wherein the rAAV comprises at least one transgene encoding one or more gene products, and wherein the rAAV infects cells of a target tissue of the subject.

41. The method of any one of the preceding paragraphs, wherein the one or more gene products comprise a therapeutic peptide, polypeptide, siRNA, microRNA, and/or antisense oligonucleotide.

### EQUIVALENTS

While several embodiments of the present invention have been described and illustrated herein, those of ordinary skill in the art will readily envision a variety of other means and/or structures for performing the functions and/or obtaining the results and/or one or more of the advantages described herein, and each of such variations and/or modifications is deemed to be within the scope of the present invention. More generally, those skilled in the art will readily appreciate that all parameters, dimensions, materials, and configurations described herein are meant to be exemplary and that the actual parameters, dimensions, materials, and/or configurations will depend upon the specific application or applications for which the teachings of the present invention is/are used. Those skilled in the art will recognize, or be able to ascertain using no more than routine experimentation, many equivalents to the specific embodiments of the invention described herein. It is, therefore, to be understood that the foregoing embodiments are presented by way of example only and that, within the scope of the appended claims and equivalents thereto, the invention may be practiced otherwise than as specifically described and claimed. The present invention is directed to each individual feature, system, article, material, and/or method described herein. In addition, any combination of two or more such features, systems, articles, materials, and/or methods, if such features, systems, articles, materials, and/or methods are not mutually inconsistent, is included within the scope of the present invention.

The articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Other elements may optionally be present other than the elements specifically identified by the “and/or” clause, whether related or unrelated to those elements specifically identified unless clearly indicated to the contrary. Thus, as a non-limiting example, a reference to “A and/or B,” when used in conjunction with open-ended language such as “comprising” can refer, in one embodiment, to A without B (optionally including elements other than B); in another embodiment, to B without A (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

In the claims, as well as in the specification above, all transitional phrases such as “comprising,” “including,” “carrying,” “having,” “containing,” “involving,” “holding,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

Use of ordinal terms such as “first,” “second,” “third,” etc., in the claims to modify a claim element does not by itself connote any priority, precedence, or order of one claim element over another or the temporal order in which acts of a method are performed, but are used merely as labels to distinguish one claim element having a certain name from another element having a same name (but for use of the ordinal term) to distinguish the claim elements.

The terms “about” and “substantially” preceding a numerical value represent ±10% of the recited numerical value.

1. A method for delivering a transgene to central nervous system (CNS) cell of a subject, the method comprising administering to the subject a recombinant adeno-associated virus (rAAV) comprising:
(i) an isolated nucleic acid comprising a transgene encoding one or more gene products flanked by adeno-associated virus (AAV) inverted terminal repeats (ITRs); and
(ii) an adeno-associated virus (AAV) capsid protein, wherein the capsid protein comprises one or more amino acid substitutions corresponding to position P32, K39, N66, A70, G115, S149, V151, P153, A126, T205, N312, R447, T450, Q457, Q461, S492, E499, P521, S525, F533, G546, E548, K556, R585, R588, and/or A593 with reference to amino acid position numbering of a wild-type AAV2 capsid protein, wherein the wild-type AAV2 capsid protein comprises the amino acid sequence set forth in SEQ ID NO: 1.

2. The method of claim 1, wherein the capsid protein comprises an amino acid substitution corresponding to P32L, K39Q, N66S, A70V, G115A, S149F, V151A, P153S, A162S, T205A, T205S, N312S, R447K, T450A, Q457M, Q461R, S492A, E499D, P521T, S525N, F533Y, G546D, E548G, E548N, K556R, R585S, R588T, A593T, and/or A593S with reference to amino acid position numbering of a wild-type AAV2 capsid protein, wherein the wild-type AAV2 capsid protein comprises the amino acid sequence set forth in SEQ ID NO: 1.

3. The method of claim 1, wherein the capsid protein comprises amino acid substitutions at amino acid positions corresponding to positions K39, N66, A70, V151, R447, T450, Q457, S492, E499, F533, G546, E548, R585, R588, and A593 with reference to amino acid position numbering of a wild-type AAV2 capsid protein, wherein the wild-type AAV2 capsid protein comprises the amino acid sequence set forth in SEQ ID NO: 1.

4. The method of claim 1, wherein the capsid protein comprises amino acid substitutions corresponding to K39Q, N66S, A70V, V151A, R447K, T450A, Q457M, S492A, E499D, F533Y, G546D, E548G, R585S, R588T, and A593T with reference to amino acid position numbering of a wild-type AAV2 capsid protein.

5. The method of claim 1, wherein the capsid protein comprises amino acid substitutions corresponding to positions K39, S149, V151, R447, T450, Q457, S492, E499, F533, G546, E548, R585, R588, and A593 with reference to amino acid position numbering of a wild-type AAV2 capsid protein, wherein the wild-type AAV2 capsid protein comprises the amino acid sequence set forth in SEQ ID NO: 1.

6. The method of claim 5, wherein the capsid protein comprises amino acid substitutions corresponding to K39Q, S149F, V151A, R447K, T450A, Q457M, S492A, E499D, F533Y, G546D, E548G, R585S, R588T, and A593T with reference to amino acid position numbering of a wild-type AAV2 capsid protein.

7. The method of claim 1, wherein the capsid protein comprises amino acid substitutions corresponding to positions K39, A70, V151, R447, T450, Q457, S492, E499, F533, G546, E548, R585, R588, and A593 with reference to amino acid position numbering of a wild-type AAV2 capsid protein, wherein the wild-type AAV2 capsid protein comprises the amino acid sequence set forth in SEQ ID NO: 1.

8. The method of claim 7, wherein the capsid protein comprises amino acid substitutions corresponding to K39Q, A70V, V151A, R447K, T450A, Q457M, S492A, E499D, F533Y, G546D, E548G, R585S, R588T, and A593T with reference to amino acid position numbering of a wild-type AAV2 capsid protein, wherein the wild-type AAV2 capsid protein comprises the amino acid sequence set forth in SEQ ID NO: 1.

9. The method of claim 1, wherein the capsid protein comprises amino acid substitutions corresponding to positions P32, K39, V151, T205, R447, T450, Q457, S492, E499, F533, G546, E548, R585, R588, and A593 with reference to amino acid position numbering of a wild-type AAV2 capsid protein, wherein the wild-type AAV2 capsid protein comprises the amino acid sequence set forth in SEQ ID NO: 1.

10. The method of claim 9, wherein the capsid protein comprises amino acid substitutions corresponding to P32L, K39Q, V151A, T205A, R447K, T450A, Q457M, S492A, E499D, F533Y, G546D, E548G, R585S, R588T, and A593T with reference to amino acid position numbering of a wild-type AAV2 capsid protein, wherein the wild-type AAV2 capsid protein comprises the amino acid sequence set forth in SEQ ID NO: 1.

11. The method of claim 1, wherein the capsid protein comprises amino acid substitutions corresponding to positions G115, V151, A162, T205, Q461, S492, E499, P521, F533, G546, K556, R585, R588, and A593 with reference to amino acid position numbering of a wild-type AAV2 capsid protein, wherein the wild-type AAV2 capsid protein comprises the amino acid sequence set forth in SEQ ID NO: 1.

12. The method of claim 11, wherein the capsid protein comprises amino acid substitutions corresponding to G115A, V151A, A162S, T205S, Q461R, S492A, E499D, P521T, F533Y, G546D, K566R, R585S, R588T, and A593S with reference to amino acid position numbering of a wild-type AAV2 capsid protein, wherein the wild-type AAV2 capsid protein comprises the amino acid sequence set forth in SEQ ID NO: 1.

13. The method of claim 1, wherein the capsid protein comprises amino acid substitutions corresponding to positions N312, R447, T450, Q457, S492, E499, S525, F533, E548, R585, R588, and A593 with reference to amino acid position numbering of a wild-type AAV2 capsid protein, wherein the wild-type AAV2 capsid protein comprises the amino acid sequence set forth in SEQ ID NO: 1.

14. The method of claim 13, wherein the capsid protein comprises amino acid substitutions corresponding to N312S, R447K, T450A, Q457M, S492A, E499D, S525N, F533Y, E548N, R585S, R588T, and A593S with reference to amino acid position numbering of a wild-type AAV2 capsid protein, wherein the wild-type AAV2 capsid protein comprises the amino acid sequence set forth in SEQ ID NO: 1.

15. The method of claim 1, wherein the capsid protein comprises amino acid substitutions corresponding to positions P153, N312, R447, T450, Q457, S492, E499, F533, E548, R585, R588, and A593 with reference to amino acid position numbering of a wild-type AAV2 capsid protein, wherein the wild-type AAV2 capsid protein comprises the amino acid sequence set forth in SEQ ID NO: 1.

16. The method of claim 15, wherein the capsid protein comprises amino acid substitutions corresponding to P153S, N312S, R447K, T450A, Q457M, S492A, E499D, F533Y, E548N, R585S, R588T, and A593S with reference to amino acid position numbering of a wild-type AAV2 capsid protein, wherein the wild-type AAV2 capsid protein comprises the amino acid sequence set forth in SEQ ID NO: 1.

17. (canceled)

18. A method for delivering a transgene to a cell of the central nervous system (CNS) in a subject, the method comprising administering to the subject a recombinant adeno-associated virus (rAAV) comprising:
(i) an isolated nucleic acid comprising a transgene encoding one or more gene products flanked by adeno associated virus (AAV) inverted terminal repeats (ITRs); and
(ii) an AAV capsid protein comprising the amino acid sequence set forth in any one of SEQ ID NOs: 2-8.

19. The method of claim 1, wherein the administration comprises intracranial injection, optionally intrahippocampal injection, intraparenchymal injection, intravenous (IV) injection, or intracerebroventricular (ICV) injection.

20. The method of claim 1, wherein the cell of the CNS is present in the corpus callosum, cornu ammonis, fimbria, polymorph layer of the dentate gyrus, and granule cell layer of the dentate gyrus of the hippocampus of the subject.

21. (canceled)

22. A recombinant expression vector comprising:
(a) a nucleic acid encoding a polypeptide comprising the sequence as set forth in any one of SEQ ID NOs: 2-8; or
(b) a nucleic acid comprising the sequence as set forth in any one of SEQ ID NOs: 10-16.
